(19) United States
(12) Patent Application Publication
(10) Pub. No.: US 2026/0256764 A1
(43) Pub. Date: Sep. 3, 2026

(54) COMPOSITIONS AND METHODS FOR ORAL DELIVERY OF SUSTAINED RELEASE CRYSTALLINE PRX-3140

(71) Applicant: NANOPHARMACEUTICS, INC., Alachua, FL (US)

(72) Inventor: James D. TALTON, Alachua, FL (US)

(73) Assignee: NANOPHARMACEUTICS, INC., Alachua, FL (US)

(21) Appl. No.: 19/489,980

(22) PCT Filed: May 30, 2024
(86) PCT No.: PCT/US2024/031598

§ 371 Date: Dec. 4, 2025

Related U.S. Application Data
(60) Provisional application No. 63/471,059, filed on Jun. 5, 2023.
(60) Provisional application No. 63/543,114, filed on Oct. 9, 2023.

(51) Int. Cl. A61K 31/4545; A61K 9/00; A61K 9/20; A61K 9/48; A61K 31/4365; A61K 31/717

(52) U.S. Cl. CPC A61K 31/4545; A61K 9/0053; A61K 9/2027; A61K 9/205; A61K 9/4866; A61K 31/4365; A61K 31/717

(57) ABSTRACT

The present disclosure relates generally to a novel crystalline form of PRX-3140, a compound of Formula I: ##STR00001##, also referred to as 6,7-Dihydro-4-hydroxy-7-isopropyl-6-oxo-N-(3-(piperidin-1-yl)propyl)thieno[2,3-b]pyridine-5-carboxamide potassium salt or potassium 7-isopropyl-6-oxo-5-(3-piperidin-1-yl-propylcarbamoyl)-6,7-dihydro-thieno[2,3-b]pyridine-4-olate, particle sustained release delivery systems of the crystalline compound, methods of preparing such compositions, and therapeutic uses thereof. The present disclosure also relates to methods for treating diabetes and reducing average daily blood glucose by the use of PRX-3140. Crystalline PRX-3140 compositions are described in U.S. Pat. Nos. 11,725,016 and 11,993,609. Sustained-release PRX-3140 formulations and uses of the present disclosure are described in patent applications 63/471,059 and 63/543,114. The compositions described herein allow the crystalline PRX-3140 potassium salt compound to be administered by routes that are non-invasive to patients, such as by oral sustained release administration.

This application claims the benefit of priority to U.S. Provisional Patent Application No. 63/471,059, filed Jun. 5, 2023 and 63/543,114 filed Oct. 9, 2023, which are hereby incorporated by reference in their entirety.

### BACKGROUND

The present disclosure relates generally to a novel crystalline form of PRX-3140, a compound of Formula I: ##STR00001##, also referred to as 6,7-Dihydro-4-hydroxy-7-isopropyl-6-oxo-N-(3-(piperidin-1-yl)propyl)thieno[2,3-b]pyridine-5-carboxamide potassium salt or potassium 7-isopropyl-6-oxo-5-(3-piperidin-1-yl-propylcarbamoyl)-6,7-dihydro-thieno[2,3-b]pyridine-4-olate, particle sustained release delivery systems of the crystalline compound, methods of preparing such compositions, and therapeutic uses thereof. The present disclosure also relates to methods for treating diabetes and reducing average daily blood glucose by the use of PRX-3140. Crystalline PRX-3140 compositions are described in U.S. Pat. Nos. 11,725,016 and 11,993,609. Sustained-release PRX-3140 formulations and uses of the present disclosure are described in patent applications 63/471,059 and 63/543,114. The compositions described herein allow the crystalline PRX-3140 potassium salt compound to be administered by routes that are non-invasive to patients, such as by oral sustained release administration.

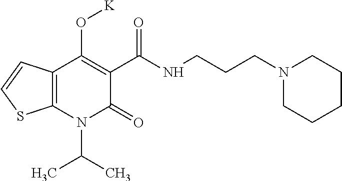

The compound of the present disclosure, referred to in the literature as PRX-3140, PRX-03140, PRX-3140 potassium salt or as PRX-3140 acid in solution (potassium ion dissociates in aqueous or physiological solution), is a selective partial agonist to the 5-Hydroxytryptamine receptor 4 (5-HT4) and a ligand for the Sigma-1 and Sigma-2 receptors. PRX-3140 is a highly selective and potent (Ki=22-37 nM) 5-HT4R agonist in radioligand binding assays with more than 100-fold difference in affinities compared with all other 5-HT receptors tested. PRX-3140 behaves as a partial agonist in cell lines expressing either the human 5-HT4aR, 5-HT4bR or 5-HT4eR isoforms, stimulating cAMP production to 30%-60% compared to 5-HT. PRX-3140 also demonstrates binding to both the Sigma-1 and Sigma-2 receptors (Ki=79-100 nM and 99-160 nM, respectively) in radioligand binding assays, but demonstrates no significant affinity for more than 50 other receptors tested including GPCRs, ion channels and receptor tyrosine kinases. The specific ligand binding to the receptors is defined as the difference between the total binding and the nonspecific binding determined in the presence of an excess of unlabelled ligand. The inhibition constants (Ki) were calculated from the Cheng Prusoff equation (Ki=IC50/(1+(L/KD)), where L=concentration of radioligand in the assay, Kd=affinity of the radioligand for the receptor and Hill coefficient=1).

PRX-3140 is being developed for Alzheimer's disease (AD) and other dementias affecting the cholinergic and/or serotonergic systems including post-traumatic stress disorder (PTSD). Extensive non-clinical studies have been completed including in vitro and in vivo pharmacology, safety pharmacology, genotoxicity as well as single and repeat dose toxicology studies in two species, rat and beagle dog. Early-stage clinical studies, including safety, tolerability, and pharmacokinetics of PRX-3140 demonstrates high oral bioavailability, as well as safety and efficacy, of the compound up to 250 milligrams. Besides its high oral bioavailability, PRX-3140 achieves a brain/serum partition ratio of 0.93 after 1 hour following oral administration in preclinical studies. PRX-3140 demonstrates high CNS penetration without inducing significant distal gastrointestinal motility observed with gastrointestinally active 5-HT4 agonists (e.g. cisapride, tegaserod).

Oral administration of drugs, such as PRX-3140, is generally preferred over intravenous administration for reasons of patient comfort and compliance. However, many drugs are variably absorbed when delivered orally. There has been substantial effort in the last decade to produce drug particles from 100 nanometers to a few hundred microns because of their improved dissolution properties and ability to be absorbed more efficiently. Through a number of experiments, the present inventors have surprisingly discovered new solid forms of PRX-3140 forms, comprising the fine particle crystalline form of the PRX-3140 potassium salt. This crystal form has unexpectedly good properties and is more suitable for formulation processing, storage, industrial production, and has better bioavailability. Compared with the known solid form of PRX-3140 described in U.S. Pat. No. 7,488,736 followed by U.S. Pat. No. 7,982,040, the fine particle crystalline form of the PRX-3140 potassium salt in present invention have at least one or more superior properties and achieve unexpected effects. Specific improvements are, for example, higher solubility in water, higher dissolution rate, better stability, lower hygroscopicity, better flowability and favorable processing and handling characteristics. Preferably, the new solid form in the present invention has improved stability.

A strong need also exists for formulations that provide sustained blood levels of PRX-3140 that avoid rapid release or delivery of PRX-3140 in a subject that may be associated with side effects for the patients. Further, a strong need also exists to ensure that a full amount of the PRX-3140 dose is available to the patient, by ensuring the drug passes through the stomach without degradation in its acidic environment. Examples of controlled and sustained release formulations include U.S. Pat. Nos. 6,984,404, 7,829,105, 7,846,459, 8,377,479, 8,394,812, 8,389,008, 8,501,232, 9,040,091, 9,554,996, 11,202,788, 11,291,673, as well as patent application 2022/0175702. In particular, there is a need for treatment of patients with Alzheimer's disease (AD) and other dementias affecting the cholinergic and/or serotonergic systems including post-traumatic stress disorder (PTSD) that have reduced interactive effect with other medications or their metabolism. Further factors decreasing the risk of clinical success with a once daily formulation in AD includes high prevalence of gastrointestinal complications having impact on the gastrointestinal parameters including transit times, pH, bacterial composition and other functions of the Gl system. These complications include nausea, vomiting, and very frequently diarrhea.

Diabetes mellitus is an inflammatory metabolic disorder marked by chronic high blood glucose levels (hyperglycemia) due to insufficient insulin activity. Insulin, produced by the pancreas's R cells, facilitates glucose utilization, protein synthesis, and glycogen formation for energy storage. Glycogen stores glucose and can be converted back as needed. In normal conditions, insulin is secreted at basal and increased rates in response to glucose, maintaining metabolic equilibrium through glucose-to-glycogen conversion. Diabetes mellitus encompasses Type I (insulin-dependent, IDDM) and Type II (non-insulin-dependent, NIDDM) diabetes. In Type I, there's an insulin deficiency, requiring insulin replacement therapy. Type II initially involves insulin resistance and may be managed with diet, lifestyle changes, and oral medications, but insulin may be needed later to control hyperglycemia and prevent complications related to high glucose levels and inflammation. The three cytokines that seem to be implicated in the inflammation of pancreatic beta cells in IDDM are interferon gamma (IFN-gamma) and the innate inflammatory cytokines TNF-alpha and IL-1beta. Further evidence also suggests that cortisol and other glucocorticoids that normally suppress insulin release are modulated by the sigma-1 receptor, leading to improved diabetic control.

The need exists, therefore, for methods for reducing inflammation and diabetes, including improving the average daily blood glucose. Surprisingly, it has been discovered that oral PRX-3140 reduces blood glucose levels in a dose dependent fashion. A strong need exists for formulations that provide sustained blood levels of PRX-3140 that avoid rapid release or delivery of PRX-3140 in a subject that may be associated with side effects for the patients. Further, a strong need also exists to ensure that a full amount of the PRX-3140 dose is available to the patient, by ensuring the drug passes through the stomach without degradation in its acidic environment. In particular, there is a need for treatment of patients with diabetes with high blood glucose that have reduced interactive effect with other medications or their metabolism. Additional effects of improved insulin and glucose levels include improved responses to glucocorticoid therapies and weight control, including obesity.

### SUMMARY OF THE INVENTION

The present disclosure addresses this need by providing crystalline PRX-3140 potassium salt preparations relating to compositions of the compound, methods for preparation and treatment. In one embodiment, the present disclosure provides novel methods of preparing sustained-release preparations of the compound of Formula I, ##STR00001## shown in FIG. 1:

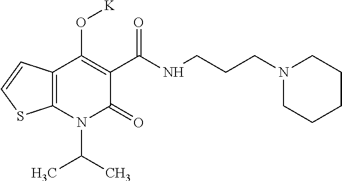

microcrystalline cellulose; and at least one sustained-release polymer chosen from hydroxy-propyl-methyl-cellulose with a number average molecular weight of about 86,000 to about 220,000 daltons, polyvinyl acetate 80% (weight average molecular weight of about 450,000 daltons)/polyvinyl pyrrolidone (weight average molecular weight of about 55,000 daltons), and xanthan gum; wherein greater than 90% by weight of the compound of Formula I is the crystalline form of Form I characterized by an x-ray powder diffraction pattern comprising major peaks at 22.3+/−0.3 degrees, 25.3+/−0.3 degrees, and 5.4+/−0.3 degrees two theta, and wherein release of the compound of Formula I from the oral dosage form defined by in vitro dissolution is at most 50% as measured by reverse-phase high performance liquid chromatography (HPLC) at the 2 hour time point and at least 50% as measured by HPLC at the 12 hour time point, wherein the dissolution of the oral dosage form is performed according to general procedures of the United States Pharmacopeia (USP) II dissolution test (paddle method) or USP I (basket method) in water or simulated gastric fluid at pH 1.2 from hours 0 to 2 and in simulated intestinal fluid at pH 6.8 after 2 hours and a rotation of 50 revolutions per minute. In certain embodiments, the present disclosure provides the oral dosage form is in the form of a capsule or tablet. In other embodiments, tablet is coated. In a further embodiment, the present disclosure provides the oral dosage form weight ratio of microcrystalline cellulose to the at least one sustained-release polymer is between about 1:3 and about 3:1. In another embodiment, the present disclosure provides a sustained release oral dosage form, wherein the weight ratio of microcrystalline cellulose to the at least one sustained-release polymer is between about 1:1 and about 3:1. In another embodiment, the present disclosure provides a sustained release oral dosage form of any one of claims 1-6, wherein the oral dosage form comprises between about 2 weight % and about 10 weight % of the compound of Formula I. In another embodiment, the present disclosure provides a sustained release oral dosage form, wherein the oral dosage form comprises between about 2 weight % and about 5 weight % of the compound of Formula I. In another embodiment, the present disclosure provides a sustained release oral dosage form, wherein the oral dosage form comprises about 3.3 weight % of the compound of Formula I. In another embodiment, the present disclosure provides a sustained release oral dosage form, wherein the oral dosage form further comprises:

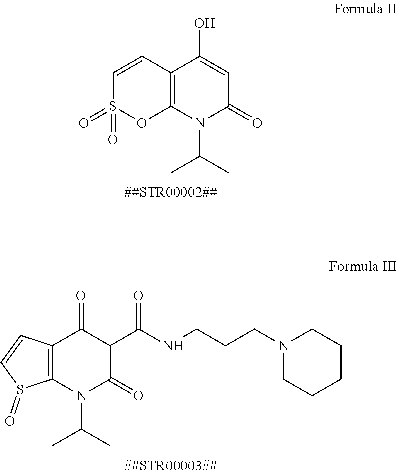 [SMILES: CC(C)n1c2c(c(O)cc1=O)C=CS(=O)(=O)O2 ; CC(C)N1C(=O)C(C(=O)NCCCN2CCCCC2)C(=O)C2=C1S(=O)C=C2]

In another embodiment, the present disclosure provides a sustained release oral dosage form, wherein the oral dosage form comprises less than 0.5 weight % of the compound of Formula II, if present, and less than 0.5 weight % of the compound of Formula III, if present.

The present invention also describes a method for treating a disease in a human in need thereof comprising administering to the human at least once daily of a therapeutically effective amount of a pharmaceutical composition comprising crystalline PRX-3140 potassium salt to achieve an average concentration at steady state (Cavg) of at least 1 ng/ml of PRX-3140 free acid in the blood. In some embodiments, the method for treating a disease in a human in need thereof includes crystalline PRX-3140 potassium salt present in the pharmaceutical composition in an amount of 0.1 mg to about 250 mg. In some embodiments, the method for treating a disease in a human in need thereof includes crystalline PRX-3140 potassium salt present in the pharmaceutical composition, wherein the disease is type 2 diabetes. In some embodiments, the method for treating a disease in a human in need thereof includes crystalline PRX-3140 potassium salt present in the pharmaceutical composition, wherein the disease is type 1 diabetes. In some embodiments, the method for treating a disease in a human in need thereof includes crystalline PRX-3140 potassium salt present in the pharmaceutical composition, wherein the disease is being treated with glucocorticoid therapy. In some embodiments, the method for treating a disease in a human in need thereof includes crystalline PRX-3140 potassium salt present in the pharmaceutical composition, wherein the disease is obesity.

In some embodiments, a composition comprising a crystalline form of a compound of Formula I: ##STR00001##, or PRX-3140 potassium salts or polymorphs or salts thereof, and wherein the crystalline Form I, characterized by an x-ray powder diffraction pattern comprising major peaks at 22.3+/−0.3 degrees, 25.3+/−0.3 degrees and 5.4+/−0.3 degrees two theta, and optionally further comprising at least one peak selected from 25.8+/−0.3 degrees, 15.9+/−0.3 degrees and 29.9+/−0.3 degrees two theta. In some embodiments, the x-ray powder diffraction pattern further comprises at least one peak selected from 21.6+/−0.3 degrees, 16.5+/−0.3 degrees and 20.3 degrees two theta. The x-ray powder diffraction pattern may further comprise peaks at 21.3+/−0.3 degrees, 17.1+/−0.3 degrees, 16.3+/−0.3 degrees, 33.1+/−0.3 degrees, 45.6+/−0.3 degrees and 13.7+/−0.3 degrees two theta. In some embodiments, crystalline Form I is characterized by an x-ray powder diffraction pattern substantially as set forth in FIGS. 3A and 3B. In some embodiments, greater than 90% by weight of the compound of Formula I in the composition may be crystalline Form I. In some embodiments, the compound of Formula I: ##STR00001## is Form I and is present in an amount ranging from about 0.01% to about 99.99% by mass of the composition. In some embodiments, the composition has an average diameter of less than about 1 mm, 0.5 mm, or 0.3 mm. In some embodiments, the compound is stable for at least 12 months at 5 and 60% relative humidity or at 25 and 60% relative humidity. In further embodiments, the formation of degradation products is less than 0.5 weight % per year at 5 and 60% relative humidity or at 25 and 60% relative humidity. In additional embodiments, the compound degradation products are Formula II: ##STR00002 ##: 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##: [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1)4-thieno[2,3-b]pyridine-5-carboxamide. In further embodiments, the composition is white or light brown color when the composition contains an amount of Formula II: ##STR00002 ##: 5-hydroxy-8-(methylethyl)-8-hydro-1,2-oxathiino[6.5 -b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##: [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1λ4-thieno[2,3-b]pyridine-5-carboxamide less than 0.5 weight %.

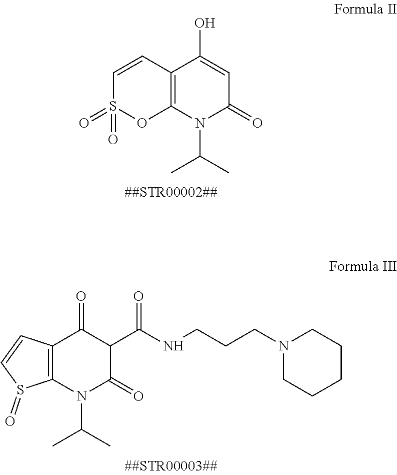 [SMILES: CC(C)n1c2c(c(O)cc1=O)C=CS(=O)(=O)O2 ; CC(C)N1C(=O)C(C(=O)NCCCN2CCCCC2)C(=O)C2=C1S(=O)C=C2]

In certain embodiments, a particulate delivery system (PDS) is described comprising a crystalline form of a compound of Formula I: ##STR00001 ## and at least one pharmaceutically acceptable excipient. In certain embodiments, the x-ray powder diffraction pattern further comprises at least one peak selected from 22.3+/−0.3 degrees, 25.3+/−0.3 degrees and 5.4+/−0.3 degrees two theta. In certain embodiments, the x-ray powder diffraction pattern further comprises at least one peak selected from 25.8+/−0.3 degrees, 15.9+/−0.3 degrees and 29.9+/−0.3 degrees two theta. In certain embodiments, the x-ray powder diffraction pattern further comprises at least one peak selected from 21.6+/−0.3 degrees, 16.5+/−0.3 degrees and 20.3 degrees two theta. In certain embodiments, the x-ray powder diffraction pattern further comprises peaks at 25.8+/−0.3 degrees, 15.9+/−0.3 degrees, 29.9+/−0.3 degrees, 21.6+/−0.3 degrees, 16.5+/−0.3 degrees and 20.3 degrees two theta. In certain embodiments, the crystalline form of a compound of Formula I is characterized by an x-ray powder diffraction pattern substantially as set forth in FIG. 3A or FIG. 3B. In addition, crystalline Form I in a matrix of sustained release polymers hydroxypropyl methylcellulose (Methocel K100M) and xanthan gum are characterized by an x-ray powder diffraction pattern substantially as set forth in FIGS. 13A and 13B, respectively. In certain embodiments, the particulate delivery system contains greater than 90% by weight of the compound of Formula I: ##STR00001## is Form I. In certain embodiments, the particulate delivery system contains the crystalline compound of Formula I: ##STR00001## is Form I and is present in an amount ranging from about 0.01% to about 99.99% by mass, about 10% to about 90% by mass, or about 10% to about 50% by mass. In certain embodiments, the particulate delivery system is formulated for oral, parenteral, or topical delivery. In certain embodiments, the particulate delivery system is formulated for oral delivery as a tablet, a caplet, a capsule, or a pill. In certain embodiments, the particulate delivery system has an average diameter of less than about 1 mm, 0.5 mm, or 0.3 mm. In certain embodiments, the pharmaceutically acceptable excipient is a polymer, a water-soluble polymer, and is chosen from starch, cellulose, or polyethylene glycol. In certain embodiments, the particulate delivery system includes a second excipient and is chosen from magnesium stearate, stearic acid, hydroxypropyl-beta cyclodextrin, silicon dioxide, mannitol, hydroxypropyl methylcellulose, polyvinyl acetate/povidone, or xanthan gum. In further embodiments, the second excipient is a sugar. In certain embodiments, the particulate delivery system is formulated for oral administration and may comprise 0.01 mg to 200 mg of the compound. In certain embodiments, the particulate delivery system contains the compound and is stable for at least 12 months at 5 and 60% relative humidity or at 25 and 60% relative humidity. In further embodiments, the formation of degradation products is less than 0.5 weight % per year at 5 and 60% relative humidity or at 25 and 60% relative humidity. In further embodiments, the compound degradation products are Formula II: ##STR00002 ##: 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##: [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1λ4-thieno[2,3-b]pyridine-5-carboxamide. In further embodiments, the particulate delivery system is white or light brown color when the composition contains an amount of Formula II: ##STR00002 ##: 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##: [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1)4-thieno[2,3-b]pyridine-5-carboxamide less than 0.5 weight %. In further embodiments, the sustained release oral dosage form comprises less than 0.5 weight % of the compound of Formula II, if present, and less than 0.5 weight % of the compound of Formula III, if present.

The present invention also describes methods method of making the particulate delivery system (PDS) of the compound of the composition, comprising: blending the composition together with an excipient to form a mixture; processing said mixture to form coarse particles having an average diameter ranging from about 0.1 mm to about 5 mm; and grinding or milling said coarse particles to form particles having an average diameter less than about 0.5 mm. In a further embodiment, present invention also describes a method of making the particulate delivery system of the compound of the composition, comprising: blending the composition together with a polymer to form a mixture; processing said mixture to form coarse particles having an average diameter ranging from about 0.1 mm to about 5 mm; and jet-milling said coarse particles to form particles having an average diameter less than about 1 micrometers.

In certain aspects, the present disclosure provides a method of treating Alzheimer's disease (AD) and other dementias affecting the cholinergic and/or serotonergic systems, comprising administering an effective amount of the composition of the compound to a patient in need thereof. In a further embodiment, the present disclosure provides a method of treating post-traumatic stress disorder (PTSD), comprising administering an effective amount of the composition of the compound to a patient in need thereof.

In certain embodiments, the present disclosure provides a method of treating Alzheimer's disease (AD) and other dementias affecting the cholinergic and/or serotonergic systems in a subject, the method comprising administering to the subject a composition comprising a crystalline form of a compound of Formula I: ##STR00001##, wherein at least 90% by weight of the compound of Formula I in the composition is the crystalline form of the compound is Form I, characterized by an x-ray powder diffraction pattern comprising major peaks at 22.3+/−0.3 degrees, 25.3+/−0.3 degrees and 5.4+/−0.3 degrees, and wherein the composition is prepared by blending the composition together with an excipient to form a mixture; processing said mixture to form coarse particles having an average diameter ranging from about 0.1 mm to about 5 mm; and grinding or milling said coarse particles to form particles having an average diameter less than about 500 micrometers.

In another aspect, the present disclosure provides a method of making a composition comprising crystalline fine particle Form I of PRX-3140 potassium salt, the method comprising:
blending crystalline fine particle Form I of PRX-3140 potassium salt together with an excipient to form a mixture;
processing said mixture to form coarse particles having an average diameter ranging from about 0.1 mm to about 5 mm; and
grinding said coarse particles to form particles having an average diameter ranging from about 0.1 micrometers to about 0.5 mm.

In another embodiment, the present disclosure provides a crystal form of a compound of Formula I: ##STR00001 ##.

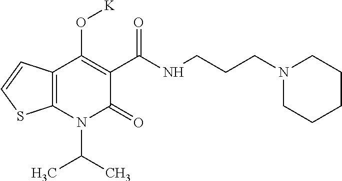

wherein Form I is described with the x-ray powder diffraction pattern further comprises at least one peak selected from 22.3+/−0.3 degrees, 25.3+/−0.3 degrees, 5.4+/−0.3 degrees, 25.8+/−0.3 degrees, 15.9+/−0.3 degrees, 29.9+/−0.3 degrees, 21.6+/−0.3 degrees, 16.5+/−0.3 degrees and 20.3 degrees two theta,
wherein greater than 90% by weight of the compound of Formula I: ##STR00001## is Form I.

In another embodiment, the present disclosure provides a crystal form of a compound of Formula I: ##STR00001##.

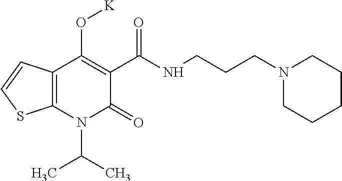

wherein the crystalline Form I, characterized by an x-ray powder diffraction pattern further comprises at least one peak selected from 22.3+/−0.3 degrees, 25.3+/−0.3 degrees, 5.4+/−0.3 degrees, 25.8+/−0.3 degrees, 15.9+/−0.3 degrees, 29.9+/−0.3 degrees, 21.6+/−0.3 degrees, 16.5+/−0.3 degrees and 20.3 degrees two theta,
wherein greater than 90% by weight of the compound of Formula I: ##STR00001## is Form I,
wherein the composition has an average diameter of less than about 500 μm,
wherein the compound is stable for at least 12 months at 5 and 60% relative humidity or at 25 and 60% relative humidity.

In another embodiment, the present disclosure provides methods of making the particulate delivery system of the compound of the composition, comprising: blending the composition together with an excipient to form a mixture; processing said mixture to form coarse particles having an average diameter ranging from about 0.1 mm to about 5 mm; and grinding or milling said coarse particles to form particles having an average diameter less than about 0.5 mm. In a further embodiment, present invention also describes a method of making the particulate delivery system of the compound of the composition, comprising: blending the composition together with a polymer to form a mixture; processing said mixture to form coarse particles having an average diameter ranging from about 0.1 mm to about 5 mm; and jet-milling said coarse particles to form particles having an average diameter less than about 1 micrometers.

In another series of embodiments, a sustained-release (SR) oral dosage form is described comprising a crystalline form of a compound of Formula I: ##STR00001 ## and at least one pharmaceutically acceptable excipient. In certain embodiments, the SR dosage form contains greater than 90% by weight of the compound of Formula I: ##STR00001## is Form I. In certain embodiments, the SR dosage form contains the crystalline compound of Formula I: ##STR00001## is Form I and is present in an amount ranging from about 0.01% to about 99.99% by mass, about 10% to about 90% by mass, or about 10% to about 50% by mass. In certain embodiments, the SR dosage form is formulated for oral, parenteral, or topical delivery. In certain embodiments, the SR dosage form is formulated for oral delivery as a tablet, a caplet, a capsule, or a pill. In certain embodiments, the pharmaceutically acceptable excipient in the SR dosage form is a polymer, a water-soluble polymer, and is chosen from starch, cellulose, gum, or polyethylene glycol. In certain embodiments, the SR dosage form includes a second excipient and is chosen from magnesium stearate, stearic acid, hydroxypropyl-beta cyclodextrin, silicon dioxide, mannitol, hydroxypropyl methylcellulose, polyvinyl acetate/povidone, or xanthan gum. In certain embodiments, the SR dosage form is formulated for oral administration and may comprise 0.01 mg to 200 mg of the compound. In certain embodiments, the SR dosage form contains the compound and is stable for at least 12 months at 5 degrees Celsius and 60% relative humidity or at 25 degrees Celsius and 60% relative humidity. In further embodiments, the formation of degradation products is less than 0.5 weight % per year at 5 degrees Celsius and 60% relative humidity or at 25 degrees Celsius and 60% relative humidity. In further embodiments, the compound degradation products are Formula II: ##STR00002 ##: 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##: [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1)4-thieno[2,3-b]pyridine-5-carboxamide. In further embodiments, the SR dosage form is white or light brown color when the composition contains an amount of Formula II: ##STR00002 ##: 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##: [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1λ4-thieno[2,3-b]pyridine-5-carboxamide less than 0.5 weight %. In further embodiments, the SR dosage form has at least two percentage dissolution parameters of PRX-3140 selected from: a percentage dissolution of less than 50% at 2 hours and a percentage dissolution of at least 50% at 12 hours, as measured at 37 degrees Celsius in water or simulated gastric fluid at pH 1.2 from hours 0 to 2 and in simulated intestinal fluid at pH 6.8 after 2 hours.

In another embodiment, the present disclosure provides a sustained release composition comprising: PRX-3140, and a sustained release agent, wherein the compound is PRX-3140, a polymorph of PRX-3140, a salt of PRX-3140, or a combination thereof, and wherein the sustained release agent is selected from the group consisting of hydroxyalkyl cellulose, hydroxyethyl cellulose, hydroxypropyl cellulose, hydroxypropylmethyl cellulose, microcrystalline cellulose, or a gum. In another embodiment, the sustained release excipient is selected from the group consisting of a cellulose, a gum, an acrylic resin, or a combination thereof. In another embodiment, the sustained release agent is a hydroxyalkyl cellulose, a hydroxyethyl cellulose, a hydroxypropyl cellulose, a hydroxypropylmethyl cellulose, or a combination thereof. In another embodiment, the sustained release agent is hydroxypropylmethyl cellulose, also known as IPMC or hypromellose. In addition, crystalline Form I in a matrix of sustained release polymers K100M IPMC and xanthan gum is characterized by an x-ray powder diffraction pattern substantially as set forth in FIGS. 13A and 13B.

In another embodiment, the sustained release composition comprises at least 1% w/w, at least 10% w/w, at least 20% w/w, at least 25% w/w, at least 30% w/w, at least 35% w/w, or at least 40% w/w of the sustained release agent. In another embodiment, the sustained release composition comprises from 1% to 99% w/w of the sustained release agent. In another embodiment, the sustained release composition comprises from 5% to 90% w/w of the sustained release agent. In another embodiment, the sustained release composition comprises from 5% to 70% w/w of the sustained release agent. In another embodiment, the sustained release composition comprises from 10% to 40% w/w of the sustained release agent. In another embodiment, the sustained release composition comprises from 15% to 40% w/w of the sustained release agent. In another embodiment, the sustained release agent comprises from 0.25% to 10% w/w, from 0.5% to 8% w/w, or from 1% to 4% w/w of the compound.

In another embodiment, the sustained release composition is formulated for oral administration as a tablet or capsule with at least 90% w/w, at least 95% w/w, or at least 99% w/w of the compound as Form I of PRX-3140. In certain embodiments, the particulate delivery system is formulated for oral administration as a tablet or capsule and may comprise 0.01 mg to 200 mg of the compound as Form I of PRX-3140 potassium salt.

In another embodiment, the sustained release composition is formulated for oral administration as a tablet or capsule and the composition further comprises one or more excipients including a binder, a lubricant, or a combination thereof. In another embodiment, the binder is a monosaccharide, a disaccharide, a starch, a polyhedric alcohol, mannitol, xylitol, sorbitol, lactose, a polyethylene glycol, a gum such as xanthan gum, alginic acid, polyvinyl pyrrolidone, methyl cellulose, polyvinyl acetate, hydroxypropylmethyl cellulose, crystalline cellulose, microcrystalline cellulose, silicified microcrystalline cellulose, calcium carbonate, calcium phosphate, sodium carbonate, sodium phosphate, anhydrous dibasic calcium phosphate, talc, a dextrate, kaolin, mannitol, silicic acid, sorbitol, or a combination thereof. In another embodiment, the binder is microcrystalline cellulose. In another embodiment, the sustained release composition formulated for oral administration as a tablet or capsule comprises from 1% to 99% w/w of the binder. In another embodiment, the sustained release composition formulated for oral administration as a tablet or capsule comprises from 30% to 97% w/w of the binder. In another embodiment, the sustained release composition formulated for oral administration as a tablet or capsule comprises from 40% to 75% w/w of the binder. In another embodiment, In another embodiment, the sustained release composition formulated for oral administration as a tablet or capsule comprises from 50% to 75% w/w of the binder.

In another embodiment, the lubricant is stearic acid, calcium stearate, magnesium stearate, zinc stearate, potassium stearate, hydrogenated vegetable oil, mineral oil, light mineral oil, glycerin, sorbitol, mannitol, a glycol, polyethylene glycol, sodium lauryl sulfate, magnesium lauryl sulfate, macrogol, talc, ethyl oleate, ethyl laureate, agar, waxes, or a combination thereof. In another embodiment, the lubricant is magnesium stearate.

In another embodiment, the sustained release composition formulated for oral administration as a final dosage form such as a tablet or capsule comprises further an enteric coating enclosing the tablet or capsule comprising the compound and the sustained release agent, wherein the enteric coating comprises: a sustained-release agent; a plasticizer; an anti-tacking agent; or a combination thereof. In another embodiment, the enteric coating is from 4% to 40% w/w of the sustained-release composition. In another embodiment, the enteric coating is from 5% to 30% w/w of the composition. In another embodiment, the enteric coating is from 5% to 25% w/w of the particulate delivery system formulated for oral administration as a tablet or capsule comprises an enteric coating from 5% to 20% w/w of the composition. In another embodiment, the final dosage form releases at least about 50 percent of the drug within about one hour, and preferably at least about 80 percent of the drug within about one or two hours, after the end of the lag time provided by the enteric-coating. In another embodiment, the enteric coating agent provides a lag time of about one to about two hours after oral administration. In another embodiment, the enteric coating agent releases at least 50 percent of the drug in the colon. In another embodiment, the enteric coating agent is a polymethacrylate.

In another embodiment, the present disclosure provides a sustained release composition comprising a tablet or capsule and an enteric coating enclosing the tablet or capsule, wherein the composition comprises: PRX-3140; and a sustained release agent, and wherein the enteric coating comprises: a delayed release agent; a plasticizer; an anti-tacking agent; or a combination thereof, wherein the compound is PRX-3140, a polymorph of PRX-3140, a salt of PRX-3140, or a combination thereof, wherein the sustained release agent is selected from the group consisting of hydroxyalkyl cellulose, hydroxyethyl cellulose, and hydroxypropyl cellulose. In another embodiment, the sustained release composition comprising a tablet or capsule and an enteric coating enclosing the tablet or capsule, comprising a core comprising a therapeutically effective amount of a drug, the sustained-release material comprising one or more natural or synthetic gums, the gums comprising from about 5 percent to about 50 percent of the tablet or capsule by weight, the enteric-coating delaying the release of the drug from the dosage form such that after oral administration to a human at least 50% of said drug is released in the colon.

In another embodiment, the present disclosure provides a sustained release composition comprising a tablet or capsule and an enteric coating enclosing the tablet, wherein the tablet comprises PRX-3140 and hypromellose, wherein the compound comprises at least 1% PRX-3140 by weight, and wherein the enteric coating comprises a poly(meth)acrylate polymer and triethyl citrate.

In another embodiment, the enteric coating further comprises talc. In another embodiment, the tablet comprises at least 20% w/w hypromellose relative to the total tablet weight. In another embodiment, the tablet comprises from 1% to 40% w/w compound relative to the total tablet weight. In another embodiment, the enteric coating comprises from 60% to 80% w/w poly(meth)acrylate polymer relative to the total enteric coating weight. In another embodiment, the enteric coating comprises no more than 10% w/w triethyl citrate relative to the total enteric coating weight. In another embodiment, the enteric coating comprises from 10% to 40% w/w talc relative to the total enteric coating weight.

In another embodiment, the present disclosure provides a method of preparing a sustained release composition comprising: combining and mixing: PRX-3140, a polymorph of PRX-3140, a salt of PRX-3140, and a combination thereof; a sustained release agent; a binder; and a lubricant; and dry compressing the compound, the at least one sustained release agent, the at least one binder, and the at least one lubricant to form a core tablet or filled capsule.

In another embodiment, the method further comprises combining and mixing: at least one sustained-release agent; at least one plasticizer; and at least one anti-tacking component or anti-adherent component, thereby forming a coating solution; and substantially coating the core tablet or filled capsule with the coating solution, thereby forming a functional coating.

In another embodiment, the present disclosure provides a method of administering a sustained release composition comprising to a subject in need thereof, the method comprising orally administering the sustained release composition to the subject in need thereof, wherein the sustained release composition comprises PRX-3140 and a sustained release agent, wherein the compound is PRX-3140, a polymorph of PRX-3140, a salt of PRX-3140, or a combination thereof, and wherein the sustained release composition is formulated such less than 50% of the compound dissolves 2 hours after administration and at least 50% of the compound dissolves within 12 hours after administration.

In another embodiment, the present disclosure provides a method of administering a sustained release composition comprising to a subject in need thereof, the method comprising orally administering the sustained release composition to the subject in need thereof, wherein the sustained release composition comprises PRX-3140 and a sustained release agent, wherein the compound is PRX-3140, a polymorph of PRX-3140, a salt of PRX-3140, or a combination thereof, and wherein the compound exhibits a time to maximum observed concentration of least 2 hours, at least 4 hours, at least 6 hours, at least 8 hours, at least 10 hours, or at least 24 hours of PRX-3140 acid in the blood, serum or plasma of the subject.

In another embodiment, the present disclosure provides a method of treating a subject in need thereof, wherein the subject has a disorder or is at risk of having the disorder, the method comprising administering to the subject the sustained release composition of the present disclosure.

In another embodiment, the present disclosure provides a method of treating a subject in need thereof, wherein the subject has a disorder or is at risk of having the disorder, the method comprising administering to the subject a sustained release composition comprising: PRX-3140, and a sustained release agent, wherein the compound is PRX-3140, a polymorph of PRX-3140, a salt of PRX-3140, or a combination thereof, and wherein the sustained release agent is selected from the group consisting of hydroxyalkyl cellulose, hydroxyethyl cellulose, hydroxypropyl cellulose, hydroxypropyl-methyl-cellulose or a gum.

In another embodiment, the sustained release composition is administered once per day, twice per day, every 2 days, every 3 days, every 4 days, every 5 days, every 6 days, or every 7 days. In another embodiment, from 0.1 mg to about 250 mg, from 1 mg to about 150 mg, from 1 mg to about 100 mg, from 1 mg to about 50 mg, or from 1 mg to about 20 mg of the sustained release composition of Form I of PRX-3140 potassium salt is administered to the subject per dose.

In yet another embodiment, the sustained-release dosage forms described herein provide a Cmax of less than 50% of the IR “drug-in-capsule” dosage form of the plasma concentration curve/time curve of PRX-3140 acid based on a single dose or at steady-state. In another aspect, the present invention is also directed to a method of treating a human in need thereof, comprising administering an sustained-release dosage form comprising a therapeutically effective amount of PRX-3140 or a pharmaceutically acceptable salt thereof in a sustained release delivery system, wherein the dosage form provides a Tmax of greater than 50% of the IR “drug-in-capsule” dosage form of the plasma concentration curve/time curve of PRX-3140 acid based on a single dose or at steady-state and maintains a plateau of a relatively constant blood serum level of PRX-3140 which does not consistently increase or decrease from time point to time point. In another aspect of the invention, the sustained-release dosage forms described herein provide less than 50% of the fluctuation % of the IR “drug-in-capsule” dosage form of the plasma concentration.

The term “Cmax” is meant for purposes of the present invention is the calculated mean the maximum blood concentration of PRX-3140 achieved after single dose administration or multiple doses of the dosage form in patients in accordance with the present invention. The term “Cmin”, also known as the trough level, is calculated mean of the minimum blood concentration of PRX-3140 achieved after multiple doses of the dosage form in accordance with the present invention, for example on Days 3, 7 or 14, or once steady-state is reached. Both parameters may be transformed using pharmacokinetic calculation prior to correlation analysis. The term “Caverage” is calculated mean of all the blood concentrations of PRX-3140 achieved between multiple doses of the dosage form in accordance with the present invention, for example on Days 3, 7 or 14, or once steady-state is reached. The term “Tmax” is meant for purposes of the present invention to mean the elapsed time from administration of a dosage form to the time the Cmax of PRX-3140 is achieved. The term AUC (area-under-the-curve), or AUC24 following once-a-day dosing, is the calculated product of concentration and time that takes into account all concentrations seen in the dosing interval, for example 24 hours for AUC24. The term % fluctuation, or the peak-to-trough fluctuation, is calculated as the ratio of the steady-state Cmax to the steady-state Cmin after administration according to the recommended dosing interval, for example 24 hours for once-a-day dosing.

The term “mean” or “calculated mean” for purposes of the present invention, when used to define a pharmacokinetic value (e.g., Tmax) represents the arithmetic mean value measured across a patient population. By “sustained release” it is meant for purposes of the present invention that, once the drug is released from the formulation, it is released at a controlled rate such that therapeutically beneficial blood levels (but below toxic levels) of the medicament are maintained over an extended period of time from the start of drug release, e.g., providing a release over a time period, e.g., from about 2 to about 24 hours from the point of drug release after the lag time, onward.

The present disclosure also addresses diabetes by providing crystalline PRX-3140 potassium salt preparations relating to compositions of the compound, methods for preparation and the treatment for diabetes and high blood glucose. Provided in one embodiment is a method for reducing blood glucose in patients comprising administering to a subject an oral formulation administered one or more times a day comprising an amount of PRX-3140 sufficient to maintain an average plasma concentration of PRX-3140 acid of at least about 1 ng/ml for at least about 12 hours. In other embodiments, the average plasma concentration of PRX-3140 acid is from about 1 ng/ml to about 900 ng/ml, from about 10 ng/ml to about 900 ng/ml or from about 10 ng/ml to about 500 ng/ml. In additional embodiments, the average plasma concentration is at least about 25 ng/ml, at least about 50 ng/ml, at least about 75 ng/ml, at least about 85 ng/ml, at least about 100 ng/ml, at least about 150 ng/ml, at least about 170 ng/ml, at least about 175 ng/ml, at least about 200 ng/ml, at least about 225 ng/ml at least about 250 ng/ml, at least about 300 ng/ml, at least about 350 ng/ml, at least about 400 ng/ml, at least about 450 ng/ml, at least about 500 ng/ml at least about 550 ng/ml, or at least about 600 ng/ml. In other embodiments, the concentration of PRX-3140 is the concentration that results in a biological or therapeutic effect, e.g. reducing blood glucose or weight reduction, equivalent to that observed with a given concentration of PRX-3140. In further embodiments, the subject suffers from diabetes or impaired glucose tolerance. A further embodiment provides a method for reducing overall daily average blood glucose concentration in a subject in need thereof, for example a subject with type I, type II or gestational diabetes, comprising administering to said subject an amount of an oral formulation containing an amount of PRX-3140 sufficient to maintain an average plasma concentration of PRX-3140 acid of at least about 1 ng/ml for at least about 12 hours. Still another embodiment provides a method for reducing hemoglobin A1C (HbA1C) in a s subject an amount of an oral formulation containing an amount of PRX-3140 sufficient to maintain an average plasma concentration of PRX-3140 acid of at least about 1 ng/ml for at least about 12 hours. In some embodiments, methods disclosed herein further provide that PRX-3140 is co-administered with one or more oral diabetic agents. Such agents include, but are not limited to metformin, a sulphonylurea (SU), a thiazolidinedione (TZD) or any combination thereof. In further embodiments, methods disclosed herein provide that oral sustained-release PRX-3140 may be used to improve insulin and glucose levels leading to improved responses to glucocorticoid therapies and weight control, including obesity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is the chemical structures of PRX-3140 (Formula I: ##STR00001##), 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione (Formula II: ##STR00002 ##), and [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-λ4-thieno[2,3-b]pyridine-5-carboxamide (Formula III: ##STR00003 ##).

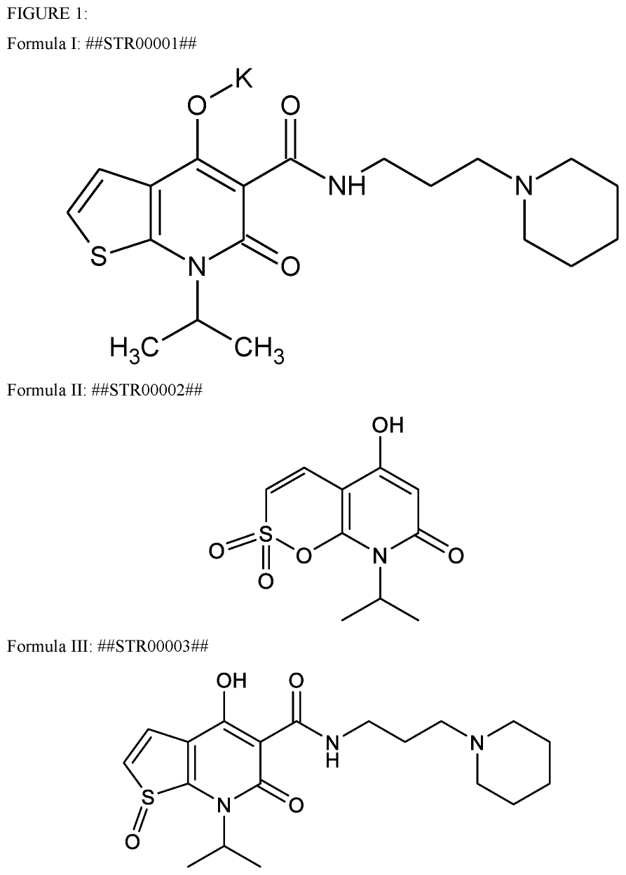

FIG. 2 shows the synthesis of crystalline fine particle Form I of PRX-3140 potassium salt.

FIG. 3 is the XRD pattern of Form I of (A) Example 1 crystalline fine particle Form I of PRX-3140 potassium salt and (B) crystalline fine particle Form I of PRX-3140 potassium salt scale-up batch.

FIG. 4 shows the crystalline fine particle Form I of PRX-3140 potassium salt photostability study HPLC chromatograms as (A) unexposed crystalline fine particle Form I of PRX-3140 potassium salt control sample and (B) crystalline fine particle Form I of PRX-3140 potassium salt photostability exposed sample.

FIG. 5 shows the crystalline fine particle Form I of PRX-3140 potassium salt peroxide degradation products Formula II: ##STR00002 ##. 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione and Formula III: ##STR00003 ##. [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1)4-thieno[2,3-b]pyridine-5-carboxamide.

FIG. 6 is the Example 3 crystalline fine particle Form I of PRX-3140 potassium salt forced degradation study (A) HPLC chromatogram of standard (Control) at 250 nm and (B) UV Spectra of Standard (Control) at 12.2 minutes. Maxima are identified at 220, 250, and 320 nm.

FIG. 7 is the (A) LC-UV chromatogram of crystalline fine particle Form I of PRX-3140 potassium salt Example 4 peroxide sample at 250 nm. Two major impurities at 9.6 and 13.3 minutes. (B) mass spectra of PRX-3140a peroxide sample at 9.6 minutes. [M+H]⁺ at 258.1 m/z, [M+H+NH3]⁺ at 275.1 m/z, [M+Na]⁺ at 280.1 m/z and [2M+Na]⁺ at 537.1 m/z. (C) mass spectra of PRX-3140b peroxide sample at 13.3 minutes. [M+H]+ at 394.2 m/z and [2M+H]+ at 787.4 m/z.

FIG. 8 is the 1H NMR of (A) the crude product of oxidation of PRX-3140 and (B) unoxidized PRX-3140 potassium salt.

FIG. 9 is the (A) UV Chromatogram PRX-3140 30% Peroxide Sample at 250 nm. (B) UV Spectra of Standard (Control) at 12.5 minutes. Maximums at 220, 250, and 320 nm.

FIG. 10 is the (A) UV Spectra of PRX-3140a at 8.9 minutes. (B) UV Spectra of PRX-3140b at 12.9 minutes.

FIG. 11 is the (A) Mass Spectra at 12.5 min for Parent Molecule, [M+H]⁺ at 378.2 m/z. (B) Mass Spectra at H2O2 Sample at 8.9 min and 70V. [M+H]+ at 258.0 m/z, [M+H+NH3]+ at 275.1 m/z, [M+Na]+ at 280.1 m/z and [2M+Na]+ at 537.1 m/z. (C) Mass Spectra at 12.9 min [M+H]+ at 394.2 m/z and [2M+H]+ at 787.4 m/z.

FIG. 12 is the HPLC chromatogram of PRX-3140. Lecithin (50:50) at (A) Time 0 initial sample and (B) 90 day sample, both at 250 nm.

FIG. 13 is the XRD pattern of (A) Example 11 crystalline fine particle Form I of PRX-3140 potassium salt mixed with hydroxypropyl-methyl-cellulose K100M (Formulation 7) and (B) Example 11 crystalline fine particle Form I of PRX-3140 potassium salt mixed with xanthan gum (Formulation 9).

FIG. 14 shows graphical representation of the dissolution (mean percent dissolved over time) of PRX-3140 described in Example 14 for “drug-in-capsule” vs. compositions Formulation 2 (50% E4M), Formulation 6 (30% E4M), Formulation 7 (30% K100M), Formulation 8 (30% Kollidon SR), and Formulation 9 (30% Xanthan Gum) in the form of a sustained release capsules (uncoated) containing 10 mg of PRX-3140, as measured according to United States Pharmacopeia (USP) I method (basket). PRX-3140 release upon dissolution of the compositions was measured using high performance liquid chromatography with ultraviolet detection (HPLC-UV) up to 24 hours.

FIG. 15 shows on a semilogarithmic scale the mean serum concentrations of crystalline PRX-3140 potassium salt orally administered as a daily dose of either a 100 mg immediate-release (IR) “drug-in-capsule” or Formulation 7 sustained-release capsule from time of dose (hour 0) to 96 hours as described in Example 15.

FIG. 16 shows on a linear scale the blood glucose level (mg/dL) vs. time of day in healthy adult patient control levels (no PRX-3140) as described in Example 18.

FIG. 17 shows on a linear scale the blood glucose level (mg/dL) vs. time of day in healthy adult patients administered crystalline PRX-3140 potassium salt orally administered as a daily morning dose of 10 mg sustained-release capsule as described in Example 18.

### DETAILED DESCRIPTION

### 1. Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical, and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include 11C, 13C, and 14C.

The opened ended term “comprising” includes the intermediate and closed terms “consisting essentially of” and “consisting of.” A significant change is any detectable change that is statistically significant in a standard parametric test of statistical significance such as Student's T-test, where p<0.05.

As described herein, modified-release dosage is a mechanism that (in contrast to immediate-release dosage) delivers a drug with a delay after its administration (delayed-release dosage) or for a prolonged period of time (extended-release dosage) or to a specific target in the body (targeted-release dosage). Sustained-release dosage forms are dosage forms designed to release (liberate) a drug at a predetermined rate in order to maintain a constant drug concentration for a specific period of time with minimum side effects. This can be achieved through a variety of formulations, including using polymers that hydrate and swell over an extended period of hours compared to minutes in an immediate-release dosage form. Sustained release's definition is more akin to a “controlled release” rather than “sustained”. Sometimes these and other terms are treated as synonyms, but the United States Food and Drug Administration has in fact defined most of these as different concepts. Modified or sustained-release dosage and its variants are mechanisms used in tablets (pills) and capsules to dissolve a drug over time in order to be released more slowly and steadily into the bloodstream, while having the advantage of being taken at less frequent intervals than immediate-release (IR) formulations of the same drug. For example, orally administered extended or sustained-release morphine can enable certain chronic pain patients to take only 1-2 tablets per day, rather than needing to redose every 4-6 hours as is typical with immediate-release morphine tablets.

### II. Synthesis of Crystalline Fine Particle Form I of PRX-3140 Potassium Salt

The disclosure includes the following embodiments, which should not be construed as limiting. Rather, these embodiments are exemplary and are provided to describe the invention to those skilled in the art. Like reference numerals refer to like elements throughout.

It has been unexpectedly discovered that dissolution of 4-hydroxy-7-isopropyl-6-oxo-6,7-dihydro-thieno[2,3-b]pyridine-5-carboxylic acid (3-piperidin-1-yl-propyl)-amide in acetonitrile and addition of potassium hydroxide in water may be used to obtain crystalline 4-hydroxy-7-isopropyl-6-oxo-6,7-dihydro-thieno[2,3-b]pyridine-5-carboxylic acid (3-piperidin-1-yl-propyl)-amide potassium salt (PRX-3140 potassium salt) by heating followed by cooling. This process is distinct from the usual crystallization procedure from U.S. Pat. Nos. 7,488,736 and 7,982,040, which uses potassium tert-butoxide in water mixed with dichloromethane and ethyl acetate, and unexpectedly provides a crystalline solid containing greater than 90% the desired 4-hydroxy-7-isopropyl-6-oxo-6,7-dihydro-thieno[2,3-b]pyridine-5-carboxylic acid (3-piperidin-1-yl-propyl)-amide potassium salt (PRX-3140 potassium salt).

In one aspect, the present disclosure provides an industrially scalable process for manufacturing PRX-3140 potassium salts thereof, comprising the steps of: (A) reductive amination of methyl 2-aminothiophene-3-carboxylate with 2,2-dimethoxynpropane using sodium triacetoxyborohydride in anhydrous dichloromethane and formic acid under argon followed by work up with potassium hydroxide to obtain methyl 2-iso-propylaminothiophene-3-carboxylate (3), (B) acylation and cyclization of methyl 2-iso-propylaminothiophene-3-carboxylate (3) in pyridine and butyronitrile with methyl malonyl chloride followed by addition of sodium methoxide solution to obtain methyl 4-hydroxy-7-isopropyl-6-oxo-6,7-dihydro-thieno[2,3-b]pyridine-5-carboxylate (5), (C) amidation of methyl 4-hydroxy-7-isopropyl-6-oxo-6,7-dihydro-thieno[2,3-b]pyridine-5-carboxylate (5) with 3-piperidin-1-yl-propylamine (6), and acidification of the resulting crude PRX-3140 with hydrochloric acid to obtain 4-hydroxy-7-isopropyl-6-oxo-6,7-dihydro-thieno[2,3-b]pyridine-5-carboxylic acid (3-piperidin-1-yl-propyl)-amide hydrochloride (8), and (D) preparation of 4-hydroxy-7-isopropyl-6-oxo-6,7-dihydro-thieno[2,3-b]pyridine-5-carboxylic acid (3-piperidin-1-yl-propyl)-amide potassium salt by adding 4-hydroxy-7-isopropyl-6-oxo-6,7-dihydro-thieno[2,3-b]pyridine-5-carboxylic acid (3-piperidin-1-yl-propyl)-amide hydrochloride (8), MTBE, water, potassium hydroxide solution, and sodium bicarbonate to give 4-hydroxy-7-isopropyl-6-oxo-6,7-dihydro-thieno[2,3-b]pyridine-5-carboxylic acid (3 -piperidin-1-yl-propyl)-amide to obtain the followed by dissolution in acetonitrile and addition of potassium hydroxide in water to give crystalline 4-hydroxy-7-isopropyl-6-oxo-6,7-dihydro-thieno[2,3-b]pyridine-5-carboxylic acid (3-piperidin-1-yl-propyl)-amide potassium salt (PRX-3140 potassium salt). The crystalline fine particle Form I of PRX-3140 potassium salt is obtained by grinding the powder and sieving.

The initial step in the disclosed process is the crystallizing of an input 4-hydroxy-7-isopropyl-6-oxo-6,7-dihydro-thieno[2,3-b]pyridine-5-carboxylic acid (3-piperidin-1-yl-propyl)-amide and acetonitrile and addition of potassium hydroxide in water to provide crystalline 4-hydroxy-7-isopropyl-6-oxo-6,7-dihydro-thieno[2,3-b]pyridine-5-carboxylic acid (3-piperidin-1-yl-propyl)-amide potassium salt (PRX-3140 potassium salt) solid and a mother liquor followed by drying, wherein the crystalline solid containing greater than 90% the desired PRX-3140 potassium salt. The crystallization can be performed by dissolving the input in acetonitrile at a first temperature by heating and then cooling the solution to a second temperature to effect crystallization. The solution can be held at the second temperature for several hours to allow for adequate crystallization. For example, the input can be dissolved in acetonitrile at 45 to 50 degrees Celsius, and then the resulting solution is cooled to 0 to 5 degrees Celsius, and held at the second temperature for 0.5 hours to 10 days, preferably 2 to 4 hours. In some cases, longer holding times at the second temperature may be required. The crystalline solid and the mother liquor may be separated by filtration, decanting, aspiration, or any suitable method. The separated crystalline solid may be washed with a suitable solvent to remove impurities and can be dried with or without heat and/or reduced pressure to remove solvent. Preferably, the crystalline solid is collected by filtration, washed with solvent, and dried in vacuo to constant weight. The separated mother liquor can be concentrated in vacuo to give a solid or a non-solid and can be dried with or without heat and/or reduced pressure to remove solvent. Preferably, the concentrated mother liquor is dried in vacuo to constant weight.

It has been unexpectedly discovered that acetonitrile is a particularly useful solvent for carrying out this step. Other solvents, such as MTBE, may also be used as the second solvent. The crystallization can be performed by dissolving the input crystalline solid in the second solvent at a first temperature of 20 to 100 degrees Celsius and then cooling the solution by 20 to 100 degrees Celsius to a second temperature to effect crystallization. The solution can be held at the second temperature for several hours to allow for adequate crystallization. For example, a solid formed from the concentrated first mother liquor can be dissolved in acetonitrile from 30 to 70 degrees Celsius, preferably from 40 to 60 degrees Celsius, and then the resulting solution is cooled to −10 to 20 degrees Celsius or 0 to 10 degrees Celsius, and held at the second temperature for 0.5 hours to 10 days or 2 to 72 hours. In some cases, longer holding times at the second temperature may be required. The crystalline solid and the mother liquor may be separated by filtration, decanting, aspiration, or any suitable method. The separated crystalline solid may be washed with a suitable solvent to remove impurities and can be dried with or without heat and/or reduced pressure to remove solvent. The separated mother liquor can be concentrated in vacuo to give a solid and can be dried with or without heat and/or reduced pressure to remove solvent. Preferably, the mother liquor is separated from the crystalline solid by aspiration, concentrated and dried in vacuo to constant weight.

### III. Crystalline Forms

In certain aspects, the present disclosure provides crystalline fine particle Form I of PRX-3140 potassium salt. The present disclosure further provides pharmaceutical compositions of PRX-3140 potassium salt comprising the crystalline forms described herein. A crystalline form of PRX-3140 potassium salt may provide the advantage of bioavailability and stability, suitable for use as an active ingredient in a pharmaceutical composition. Variations in the crystal structure of a pharmaceutical drug substance or active ingredient may affect the dissolution rate (which may affect bioavailability, etc.), manufacturability (e.g., ease of handling, ability to consistently prepare doses of known strength) and stability (e.g., thermal stability, shelf life, etc.) of a pharmaceutical drug product or active ingredient. Such variations may affect the preparation or formulation of pharmaceutical compositions in different dosage or delivery forms, such as solid oral dosage forms including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, crystalline forms of PRX-3140 potassium salt may provide advantages such as: improving the manufacturing process of an active agent or the stability or storability of a drug product form of the compound or an active ingredient, and/or having suitable bioavailability and/or stability as an active agent.

The use of certain solvents and fractional crystallization methods has been found to produce different polymorphic forms of PRX-3140 potassium salt, including polymorphic Form I, which may exhibit one or more favorable characteristics described above. The processes for the preparation of the polymorphs described herein, and characterization of these polymorphs are described in greater detail below.

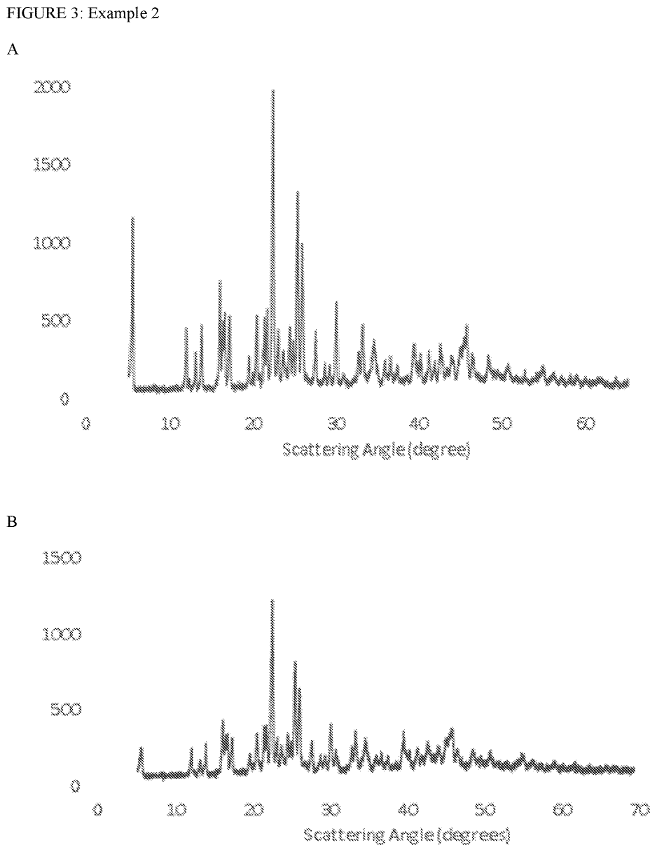

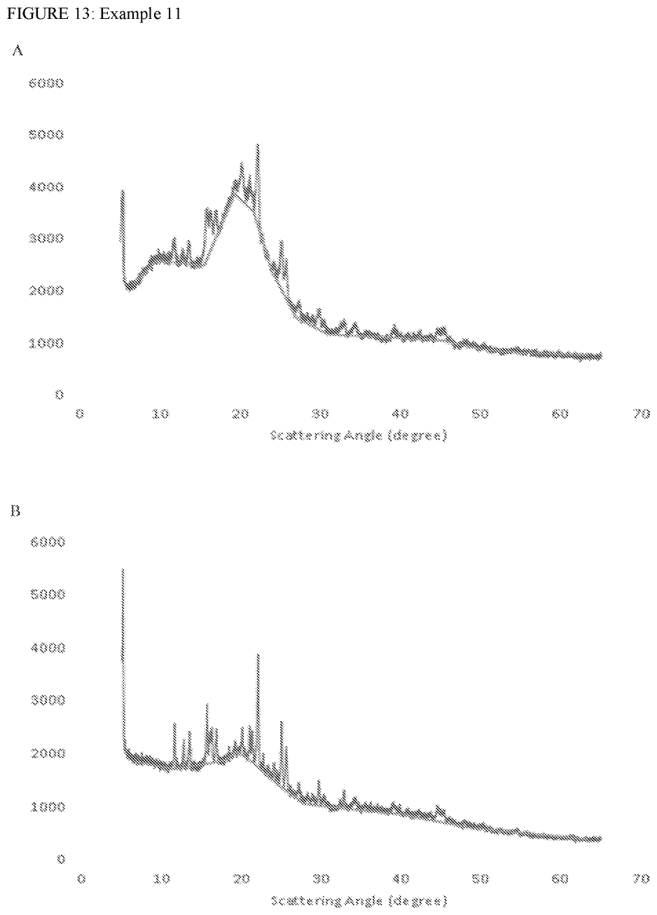

In certain embodiments, the crystalline form of a compound of Formula I is characterized by an x-ray powder diffraction pattern substantially as set forth in FIG. 3A or FIG. 3B. In addition, crystalline Form I in a matrix of sustained release polymers hydroxypropyl methylcellulose (Methocel K100M) and xanthan gum are characterized by an x-ray powder diffraction pattern substantially as set forth in FIGS. 13A and 13B, respectively. In certain aspects, the present disclosure provides polymorphic Form I of PRX-3140 potassium salt, wherein at least 90% by weight is PRX-3140 potassium salt. In some embodiments, polymorphic Form I exhibits an x-ray diffraction (XRD) pattern substantially as shown in FIG. 3A. In some embodiments, polymorphic Form I has an XRD pattern comprising at least two, at least three, at least four, at least five, or at least six of the major peaks as the XRD pattern substantially as shown in FIG. 3A. The crystalline structure of the present invention is substantially pure, unitary, and substantially free of any other crystal form or amorphous state. “Substantially pure” in the present invention when used in reference to a new crystal form means that this new crystal form comprises at least 80% (by weight) of the present compound, more preferably at least 90% (by weight), and especially at least 95% (by weight), especially at least 99% (by weight).

The term “substantially as shown in” when referring, for example, to an XRD pattern, includes a pattern that is not necessarily identical to those depicted herein, but that falls within the limits of experimental error or deviations when considered by one of ordinary skill in the art. The relative intensities of XRD peaks can vary, depending upon the particle size, the sample preparation technique, the sample mounting procedure and the particular instrument employed. The crystalline form in the present invention means that the compound is confirmed by the X-ray powder diffraction pattern characterization shown and has a unique and ordered molecular arrangement or configuration within the crystal lattice. It is well known to those skilled in the art that the experimental error depends on the instrument conditions, sample preparation and sample purity. The 2 theta angle of the peaks in the XRD pattern usually varies slightly depending on the instrument and sample. The difference in peak angle may differ by 1 degrees, 0.8 degrees, 0.5 degrees, 0.3 degrees, 0.1 degrees, etc. according to different instruments, different samples, etc. Generally, the tolerance is +/−0.2 degrees. Therefore, the difference in peak angle cannot be used as the sole criterion. The relative intensity of peaks may vary with samples, sample preparation, and other experimental conditions, so the order of peak intensities cannot be the sole or decisive factor. The influence of experimental factors such as sample height will cause the overall shift of the peak angle, which usually allows a certain shift. Therefore, those skilled in the art can understand that any crystal form having the same or similar characteristic peaks as the X-ray powder diffraction pattern of the present invention belongs to the scope of the present invention. “Single crystalline form” refers to a single crystal form as determined by X-ray powder diffraction.

Moreover, instrument variation and other factors can affect the two theta values. Accordingly, when a specified two theta angle is provided, it is to be understood that the specified two theta. angle can vary by the specified value+/−0.5 degrees, such as +/−0.4 degrees, +/−0.3 degrees, +/−0.2 degrees, or +/−0.1 degrees. As used herein, “major peak” refers to an XRD peak with a peak intensity greater than baseline, such as greater than 100 or 500 depending on the baseline noise and other test factors listed above.

In certain aspects, the present disclosure provides at least 90% by weight of PRX-3140 compound of Formula I in the composition is a crystalline form of the potassium salt. Crystalline Form I may be characterized by an x-ray powder diffraction pattern comprising major peaks at 22.3+/−0.3 degrees, 25.3+/−0.3 degrees and 5.4+/−0.3 degrees two theta, and optionally further comprising at least one peak selected from 25.8+/−0.3 degrees, 15.9+/−0.3 degrees and 29.9+/−0.3 degrees two theta. In some embodiments, the x-ray powder diffraction pattern further comprises at least one peak selected from 21.6+/−0.3 degrees, 16.5+/−0.3 degrees and 20.3 degrees two theta. The x-ray powder diffraction pattern may further comprise peaks at 21.3+/−0.3 degrees, 17.1+/−0.3 degrees, 16.3+/−0.3 degrees, 33.1+/−0.3 degrees, 45.6+/−0.3 degrees and 13.7+/−0.3 degrees two theta. In some embodiments, crystalline Form I is characterized by an x-ray powder diffraction pattern substantially as set forth in FIG. 3A. Greater than 90%, 95% or 99% by weight of the compound of Formula I in the composition may be crystalline Form I. In some embodiments, the composition comprises 0.01 mg to 200 mg of crystalline Form I, such as about 10 mg, 25 mg, 50 mg, 75 mg, 100 mg or 200 mg of crystalline Form I.

In some embodiments, a composition comprising crystalline form of PRX-3140 potassium salt comprises 0.01%, 0.05%, 0.01%, 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%1, 17%, 18%, 19% or 20% PRX-3140 potassium salt (wt %/wt %) or (w/v) of the composition. In some embodiments, a composition comprising crystalline form of PRX-3140 potassium salt comprises 0.01%, 0.05%, 0.01%, 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%1, 17%, 18%, 19% or 20% PRX-3140 potassium salt (wt %/wt %) or (w/v) of the composition.

The compounds and compositions of the present disclosure can be administered to a subject in need thereof by any route known in the art, including without limitation, oral, parenteral, topical, and intraductal delivery. Accordingly, compositions disclosed herein are formulated to be compatible with the intended route of administration.

In some embodiments, the compositions comprising crystalline form of PRX-3140 potassium salt further comprise an excipient. Such an excipient can be compatible with the intended route of administration.

### IV. Methods of Making a Particle Delivery System (PDS)

The present disclosure also provides a method of making a composition of the present disclosure comprising particles of the crystalline form of PRX-3140 potassium salt encapsulated by an excipient, the method comprising:
blending crystalline form of PRX-3140 potassium salt together with an excipient to form a mixture;
processing said mixture to form coarse particles having an average diameter ranging from about 0.1 mm to about 5 mm; and
grinding or milling said coarse particles to form particles having an average diameter less than about 500 micrometers.

In certain embodiments, the particles have an average diameter ranging from about 0.1 microns to about 0.1 mm. Particulate materials, also designated as “particles”, to be produced in accordance with this disclosure are those in which small nanometer to micrometer size particles may be desirable. Examples may include nanoparticles and microparticle forms of pharmaceuticals, including crystalline form of PRX-3140 potassium salt. The possibilities and combinations are numerous.

In one embodiment, a system for preparing a composition of the present disclosure may include a grinding the crystalline form of PRX-3140 potassium salt in a mortar and pestle or with a ball mill. In another embodiment a system for preparing a composition of the present disclosure may include a venturi-type nozzle or ‘Tee’ valve to introduce cryogenic gas to, for example, a jet mill. Without wishing to be bound by any particular theory, combinations of dry gases at cryogenic temperatures (generally below 0 degrees Celsius) before introduction into the jet mill may be used to eliminate moisture-induced agglomeration, as well as promote brittle fracture of particles upon impaction, and has been observed to act synergistically to produce a marked improvement in the particle size reduction efficiency. Cryogenic liquids suitable for use in this method include liquid argon, liquid nitrogen, liquid helium or any other liquified gas having a temperature sufficiently low to produce brittle fracture of particles. The cryogenic liquid may also prevent milling losses and thermal damage to the feed material that would otherwise be caused by the volatization or overheating of constituent ingredients.

In one embodiment, a powder is placed in a temperature-controlled vessel, such as a jacketed hopper or a screw-feeder or is frozen beforehand. The cryogenic liquid and gas inputs are opened, and the flow and temperature are set to the desired process conditions. The cryogenic gas input system, for example liquid nitrogen mixed with nitrogen gas, may be connected to a standard commercial jet mill, such as a Trost Gem-T, Trost T-15, Fluid Air Aljet, Hosikawa Alpine AS Spiral Jet Mill, Sturtevant Micronizer, or similar system, as the main carrier gas in a variety of gas input setups. Pre-run setup of the system may include attaching a temperature probe or flowmeter, such as a TSI Model 4040 Flowmeter or similar system, at the gas input or to the top of the cyclone (in place of air relief bag), setting the carrier gas on different input pressures and documenting the gas flow and temperature measurements (CFM). The milling process may be started by turning on the powder feeder and after passing powder through the milling region, the jet-milled powder is collected in the cup or similar receiver unit (typically particles ~1-10 microns) or from the bag above the cyclone (particles <1 micron), depending on the exact run conditions. Particles with diameters ranging from less than about 1 micron to about 10 microns may be produced by running the powder from the cup through the jet-mill under similar run conditions multiple times, or passes, to obtain the desired particle size.

In certain embodiments, the particles may have an average diameter ranging from about 0.1 mm (100 microns) to about 3 mm. For example, the particles may have a diameter of less than about 2.06 mm (corresponding to a 10 mesh sieve), less than about 1.68 mm (corresponding to a 12 mesh sieve), less than about 1.40 mm (corresponding to a 14 mesh sieve), less than about 1.20 mm (corresponding to a 16 mesh sieve), less than about 1.00 mm (corresponding to an 18 mesh sieve), less than about 0.853 mm (corresponding to a 20 mesh sieve), less than about 0.710 mm (corresponding to a 25 mesh sieve), less than about 0.599 mm (corresponding to a 30 mesh sieve), or less than about 0.500 mm (corresponding to a 35 mesh sieve). In some embodiments, the particles may have a diameter of less than about 300 microns and may be able to pass through a 50-mesh sieve. In certain embodiments, the particles have a diameter of about 0.6 mm or less.

In certain embodiments, the controlled or sustained-release polymer is heated prior to blending with the crystalline form of PRX-3140 potassium salt.

In some embodiments, the present disclosure provides a method of making a composition of the present disclosure comprising particles of the crystalline form of PRX-3140 potassium salt encapsulated by a controlled-release polymer using a process wherein the process is at least partially a continuous manufacturing process. The method may comprise:
blending the crystalline form of PRX-3140 potassium salt together with a controlled-release polymer to form a mixture;
heating said mixture to a temperature sufficient for extrusion of the mixture;
extruding said mixture to form coarse particles having an average diameter ranging from about 0.1 mm to about 5 mm;
cooling said coarse particles; and
processing (e.g., by milling, grinding, or crushing) said coarse particles to form particles having an average diameter less than about 0.1 mm.

In certain embodiments, the particles may have an average diameter ranging from about 0.1 mm (100 microns) to about 3 mm. For example, the particles may have a diameter of less than about 2.06 mm (corresponding to a 10 mesh sieve), less than about 1.68 mm (corresponding to a 12 mesh sieve), less than about 1.40 mm (corresponding to a 14 mesh sieve), less than about 1.20 mm (corresponding to a 16 mesh sieve), less than about 1.00 mm (corresponding to an 18 mesh sieve), less than about 0.853 mm (corresponding to a 20 mesh sieve), less than about 0.710 mm (corresponding to a 25 mesh sieve), less than about 0.599 mm (corresponding to a 30 mesh sieve), or less than about 0.500 mm (corresponding to a 35 mesh sieve). In some embodiments, the particles may have a diameter of less than about 300 microns and may be able to pass through a 50 mesh sieve. In certain embodiments, the particles may have a diameter of about 0.1 mm or less.

In certain embodiments, the controlled or sustained-release polymer may be heated prior to blending with the crystalline form of PRX-3140 potassium salt.

### V. Pharmaceutical Compositions (Final Dosage Forms)

The present disclosure further provides pharmaceutical compositions (sometimes referred to as “final dosage forms” or “FDF”) comprising compositions according to the present disclosure, including the PDS from the previous section. In certain embodiments, the FDF includes immediate and sustained-release oral dosage forms, as well as other forms for delivery of crystalline PRX-3140 potassium salt.

In some embodiments, the pharmaceutical compositions may further comprise at least one excipient (such as, e.g., a controlled-release polymer, surfactant, and/or metal salt), such as a pharmaceutically acceptable excipient. Examples of pharmaceutically acceptable excipients may be, for example, those described in Remington's Pharmaceutical Sciences by E. W. Martin, and include cellulose, starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. In some embodiments, the pharmaceutical compositions also contain pH buffering reagents, and wetting or emulsifying agents.

In some embodiments, the pharmaceutical compositions may be formulated for oral administration. In this embodiment, the pharmaceutical composition may be in the form of, for example, tablets, capsules, or other oral dosage forms. Such oral dosage forms may be prepared by conventional means. The pharmaceutical composition can also be prepared as a liquid, for example as a syrup or a suspension. The liquid can include suspending agents (e.g., sorbitol syrup, cellulose derivatives, or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring, and sweetening agents. Alternatively, the composition can be presented as a dry product for constitution with water or another suitable vehicle.

For buccal and sublingual administration, the composition may take the form of tablets or lozenges according to conventional protocols.

The pharmaceutical composition can also be formulated for rectal administration as a suppository or retention enema, e.g., containing conventional suppository bases such as PEG, cocoa butter, or other glycerides.

Conventional in vitro dissolution methods include the methods described in The United States Pharmacopeia (USP) the official public standards-setting authority for all prescription and over-the-counter medicines in USA and similar pharmacopeias for Europe and Japan. The preferred methods include the USP dissolution method I (basket) and method Il (paddle) at 50 revolutions per minute (RPM). Accordingly, it can be relevant to describe the degree of extension of the release with alternative dissolution methods, in addition, the extended formulation accordingly may be further characterized by additional dissolution methods, such as methods with different rotation speeds, different pH values, use of dissolution media simulating Gl conditions (e.g simulation of the fasted and fed state, FaSSIP and FeSSlP medias), use of additives to the dissolution medium such as SLS to increase the wettability or the solubility whereby the overall dissolution time measured is decreased (dissolution rate is increased).

In some embodiments, the pharmaceutical compositions described herein provide improved dissolution of the crystalline form of PRX-3140 potassium salt, relative to the unencapsulated crystalline form of PRX-3140 potassium salt, and/or to another dosage form (such as, e.g., a more invasive dosage form). For example, dissolution may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by a Vankel tablet dissolution apparatus approved by the United States Pharmacopeia.

In some embodiments, the pharmaceutical compositions described herein provide improved oral bioavailability of the crystalline form of PRX-3140 potassium salt, relative to the unencapsulated crystalline form of PRX-3140 potassium salt, and/or to another dosage form (such as, e.g., a more invasive dosage form). For example, absorption may be increased by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo pharmacokinetic studies in a preclinical animal model or human clinical evaluation.

In some embodiments, the pharmaceutical compositions described herein are immediate-release formulations. In such embodiments, the pharmaceutical compositions provide a more rapid onset of action of the crystalline form of PRX-3140 potassium salt, relative to the unencapsulated crystalline form of PRX-3140 potassium salt, and/or to another dosage form (such as, e.g., a more invasive dosage form). For example, the onset of action may be shortened by, e.g., at least 10%, 15%, 20%, 25%, 30%, 35%, 40%, 50%, 60%, 70%, 80%, 90%, 93%, 95%, 96%, 97%, 98%, 99%, 100%, 110%, 120%, 130%, 140%, 150%, or 200%, or by, e.g., at least 2, 3, 4, 5, 6, 7, 8, 9, 10, 20, 30, 40, 50, 100, or 1000 fold, as measured by, e.g., in vivo pharmacokinetic studies in a preclinical animal model or human clinical evaluation.

In some embodiments, the pharmaceutical compositions described herein are sustained-release formulations. In such embodiments, the pharmaceutical compositions described herein provide a more rapid onset of action of the crystalline form of PRX-3140 potassium salt.

In some embodiments, the pharmaceutical compositions described herein have reduced absorption variability, relative to the unencapsulated insoluble drug, and/or to another dosage form (such as, e.g., a more invasive dosage form).

In some embodiments, the pharmaceutical compositions described herein are associated with improved patient compliance, relative to another pharmaceutical composition comprising the crystalline form of PRX-3140 potassium salt (which may be in another dosage form, such as, e.g., a more invasive dosage form).

In some embodiments, a pharmaceutical composition of the present disclosure is formulated for oral delivery. Compositions intended for oral use may be prepared in solid or fluid unit dosage forms. In at least some embodiments, the compositions are formulated for oral delivery as tablets, caplets, capsules, pills, powders, troches, elixirs, suspensions, syrups, wafers, chewing gums, dragees, lozenges, and the like.

In some embodiments, the oral dosage forms are solid oral dosage forms such as tablets, caplets, and capsules. In some embodiments, the capsule is a hard capsule or a soft capsule. In other embodiments, the capsule is a gelatin capsule, gelatin-free capsule, a “cap-in-cap” capsule, alginate capsule, hydroxypropylmethyl cellulose (HPMC) capsule, a polyvinyl alcohol (PVA) capsule, a hypromellose capsule, or a starch capsule.

In some embodiments, an oral composition comprising the crystalline form of PRX-3140 potassium salt thereof further comprises one or more excipients. In some embodiments, an oral composition comprising the crystalline form of PRX-3140 potassium salt or a polymorph thereof further comprises one or more excipients. Accordingly, compositions designed for oral administration can be made with an inert or active excipient or with an edible carrier as disclosed herein.

In various embodiments, the composition provided herein comprises from about 1% to about 99.99%, about 5% to about 95%, about 5% to about 90%, about 10% to about 80%, about 15% to about 70%, about 20% to about 60%, from about 30% to about 95%, from about 50% to about 90%, from about 60% to about 90%, from about 60% to about 80%, or from about 70% to about 80% by weight of one or more excipients. In certain embodiments, the composition provided herein comprises about 99.99%, about 95%, about 90%, about 85%, about 80%, about 75%, about 70%, about 65%, about 60%, about 55%, or about 50% by weight of one or more excipients. In certain embodiments, the composition provided herein comprises about 99.99%, about 99%, about 98%, about 97%, about 96%, about 95%, about 94%, about 93%, about 92%, about 91%, about 90%, about 89%, about 88%, about 87%, about 86%, or about 85% by weight of one or more excipients. In certain embodiments, the composition provided herein comprises about 85%, about 84%, about 83%, about 82%, about 80%, about 79%, about 78%, about 77%, about 76%, about 75%, about 74%, about 73%, about 72%, about 71%, about 70%, about 69%, about 68%, about 67%, about 66%, or about 65% by weight of one or more excipients. In certain embodiments, the composition provided herein comprises about 55%, about 54%, about 53%, about 52%, about 51%, about 50%, about 49%, about 48%, about 47%, about 46%, or about 45% by weight of one or more excipients. In certain embodiments, the composition provided herein comprises about 30%, about 29%, about 28%, about 27%, about 26%, about 25%, about 24%, about 23%, about 22%, about 21%, or about 20% by weight of one or more excipients.

Examples of excipients that can be used in the compositions formulated for oral administration are provided herein and can include, but are not limited to, one or more of bulking agents, binders, fillers, disintegrating agents, lubricants, glidants, control release agents, enteric coatings, film-forming agents, plasticizers, colorants, sweetners, flavoring agents and the like, or any combination thereof.

Binders suitable for use in the pharmaceutical compositions provided herein include, but are not limited to, sucrose, starches such as corn starch, potato starch, or starches such as starch paste, pregelatinized starch, and starch 1500, PEG 6000, methocel, walocel HM, Luvitec, Luvicaparolactam, Avicel, SMCC, UNIPURE, gelatin, natural and synthetic gums such as acacia, sodium alginate, alginic acid, other alginates, tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, polyvinyl pyrrolidone, hydroxypropyl methyl cellulose (e.g., Nos 2208, 2906, 2910), microcrystalline cellulose, and mixtures thereof. Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. In some embodiments, the binder is a mixture of microcrystalline cellulose and sodium carboxymethyl cellulose. Suitable anhydrous or low moisture excipients or additives include AVICEL PH 103 and Starch 1500 LM.

Examples of fillers suitable for use in the pharmaceutical compositions provided herein include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose dextrates, kaolin, mannitol, silicic acid, sorbitol, starch, pregelatinized starch, and mixtures thereof.

One or more binder or filler in compositions is typically present in from about 10% to about 99% (wt %/wt %) of the composition or the dosage form. In some embodiments, binders and/or fillers in a composition comprise about 15% to 99%, about 20% to 60%, about 25% to 55%, about 30% to 50%, about 35% to 60%, about 50% to 99% (wt %/wt %) of the composition.

Disintegrants can be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. In some embodiments, the disintegrant is deep in the oral solid dosage form to delay disintegration. The amount of disintegrant used varies based upon the type of formulation, and is readily discernible to those of ordinary skill in the art.

Typical compositions comprise from 0.5% to 15% (wt %/wt %) of disintegrant. In some embodiments, compositions comprise from 1% to 5% (wt %/wt %) of disintegrant in the composition. In another embodiment, the disintegrant is 1% to 25%, 2% to 20%, 5% to 15%, 8% to 12%, or about 10% (wt %/wt %) of the composition.

Disintegrants that can be used in the pharmaceutical compositions provided herein include, but are not limited to, agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in the pharmaceutical compositions provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, magnesium stearate or potassium stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), Q7-9120 (Dow Corning), and mixtures thereof. If used at all, lubricants are typically used in an amount of less than 1% (wt %/wt %) of the compositions or dosage forms into which they are incorporated. In yet another embodiment, the lubricant is 0.1% to 3%, such as 0.5% to 1% (wt %/wt %), of the composition.

Plasticizers may be added to control the softness or pliability of oral dosage forms such as shell of a capsule, caplet or a tablet and thus, may improve the mechanical properties of the pH-sensitive materials of the coatings on the oral dosage forms. Suitable plasticizers, include, without limitation, petroleum oils (for e.g., a paraffinic process oil, a naphthenic process oil, and an aromatic process oil), squalene, squalane, plant oils, (e.g., olive oil, camelia oil, castor oil, tall oil, and a peanut oil), silicon oils, dibasic acid esters, (e.g., dibutyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, triethyl citrate, triacetin, diethylene glycol, polyethylene glycols, polypropylene glycol, phthalates, sorbitol, glycol salicylate, crotaminton, and glycerin or mixtures thereof. The amount of plasticizer may vary depending upon the chemical composition of the pharmaceutical preparation. In one embodiment, the at least one plasticizer is sorbitol, dimethyl isosorbide, or a glycerol. In another embodiment, the plasticizer is 1% to 10%, such as 3% to 5% (wt %/wt %), of the composition.

Examples of glidants include, but are not limited to, colloidal silicone dioxide, cellulose, calcium phosphate, di or tri-basic and the like.

As an example of sweeteners or sweetening agents include sucrose, saccharin, dextrose, maltose, sugar substitutes, aspartame, xylitol, mannitol, cyclamate, sucralose, maltitol, sorbitol, acesulfame K and the like.

Examples of flavoring agents include peppermint, methyl salicylate, peppermint, spearment, methyl salicylate, raspberry, red berry, strawberry, pineapple, orange, cherry and the like.

Compositions formulated for oral delivery as disclosed herein, for example, tablets, caplets, and capsules, may be coated with one or more enteric coating agent, control release agent or film forming agent to control or delay disintegration and absorption of the compositions comprising the crystalline form of the compound of Formula I thereof in the gastrointestinal tract and thereby provide a sustained action over a longer period of time. Accordingly, in some embodiments, the tablet can be an enteric tablet, the caplet can be an enteric caplet, or the capsule can be an enteric capsule. The enteric tablets, enteric caplets, or enteric capsules of the present disclosure may be prepared by techniques known in the art.

Pharmaceutical preparations disclosed herein may comprise a control release agent. Examples of control release agent suitable for use include, without limitation, pH-dependent polymers, acid-insoluble polymers, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, zein, waxes, including synthetic waxes, microcrystalline waxes, paraffin wax, carnauba wax, and beeswax; polyethoxylated castor oil derivatives, hydrogenated oils, glyceryl mono-, di-tribenates, glyceryl monostearate, glyceryl distearate, long chain alcohols, such as stearyl alcohol, cetyl alcohol, and polyethylene glycol; and mixtures thereof. In some embodiments, a time delay material such as glyceryl monostearate or glyceryl distearate may be used. In other embodiments, the controlled release reagent is a digestible waxy substance such as hard paraffin wax.

In some embodiments, compositions may comprise one or more of pH-dependent polymers such as acid insoluble polymers. The pH-dependent polymers become increasingly permeable above pH 5.0 but are impermeable at pH below 5.0 whereas acid insoluble polymers become soluble in neutral to weakly alkaline conditions. Such control release polymers target upper small intestines and colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, polyvinyl acetate 80%/polyvinyl pyrrolidone, xanthan gum, acrylic acid-methylacrylic acid copolymers (commercially available under the tradename EUDRAGIT® L and EUDRAGIT® S from Rohm America Inc., Piscataway, N.J. as a powder or a 30% aqueous dispersion; or under the tradename EASTACRYL®, from Eastman Chemical Co., Kingsport, Tenn., as a 30% dispersion). Additional examples include EUDRAGIT® L100-55, EUDRAGIT® L30D-55, EUDRAGIT® L100, EUDRAGIT® L100 12,5, EUDRAGIT® S100, EUDRAGIT® S12,5, EUDRAGIT® FS 30D, EUDRAGIT® E100, EUDRAGIT® E 12,5, and EUDRAGIT® PO. In at least one embodiment, the composition comprises EUDRAGIT® L100-55. EUDRAGIT® RS and RL and EUDRAGIT® NE and NM are also useful polymers for the purpose of this disclosure. In some embodiments, the composition comprises EUDRAGIT® L30D 55. In another embodiment, the preparation comprises EUDRAGIT® FS 30D. One of skill in the art will recognize that at least some acid insoluble polymers listed herein will also be biodegradable.

For time delay or delayed-release pharmaceutical preparations of oral dosage forms, glyceryl monostearate, glyceryl distearate, and acid-insoluble polymers, for example polymethacrylate pH-sensitive polymer-based coatings can be used, (e.g., as coating material, i.e., enteric coating agents, for enteric coating of capsules, caplets, and tablets). Commercial sources for delayed-release oral dosage forms are available, for example DRCaps made of hypromellose (HPMC) from Capsugel, USA. Such delayed-release oral dosage forms are acid-resistant and can resist acidity as seen in stomach for at least 30 min, such as for at least 1 hour, for at least 1.5 hour, or for at least 2 hours. Such delayed release oral dosage forms can release at least 40%, at least 50%, at least 60%, at least 70%, at least 80% or at least 90% of the crystalline form of the compound of Formula I thereof in the intestines (small intestines, large intestine/colon etc).

In an aspect of the present disclosure, the enteric tablets, enteric caplets, and enteric capsules may be uncoated. Hard uncoated capsules with enteric capability using intrinsically enteric capsule technology (for example, EnTrinsic Drug Delivery available from Capsugel) are suitable for the purpose of the present disclosure.

In various embodiments, the enteric tablet is a hard tablet made with free-flowing powder of the crystalline form of the compound of Formula I thereof. In various embodiments, the enteric capsule is a capsule made with free-flowing powder of crystalline form of the compound of Formula I thereof. In various embodiments, the enteric tablet is a hard tablet made with free-flowing powder of the crystalline form of the compound of Formula I. In various embodiments, the enteric capsule is a capsule made with free-flowing powder of the crystalline form of the compound of Formula I.

In some embodiments, the enteric capsule is a non-animal-based capsule, such as a hypromellose capsule (for example, commercially available self-gelling Vcaps, VCaps Plus, VCaps enteric, other enteric capsules made using Xcellodose, ENCODE colonic delivery technology, and EnTrinsic™ drug delivery technology from Capsugel). Other technologies known in the art and available commercially (for example, Qualicaps, USA, Nutrascience, USA, etc.) for the formulating enteric forms of oral solid dosage forms can also be utilized. In at least one embodiment, the capsule is an API-in-capsule, meaning that the crystalline form of the compound of Formula I or salts thereof is filled neat into the capsule. In such API-in-capsule, or “drug-in-capsule”, oral dosage forms, the active ingredient, the crystalline form of the compound of Formula I can be free flowing powders or micronized powders. When the dosage Form I is in a capsule, in at least one embodiment, the capsule can be a seamless capsule or a banded capsule.

Dissolution of the oral dosage forms disclosed herein is tested by the dissolution tests according to the current methods of USP 711. In some embodiments, the oral dosage forms disclosed herein are protected the acidic environment of the stomach and do not dissolve for at least 2 hours, at least 3 hours, at least 4 hours, at least 5 hours, 6 hours, at least 7 hours or at least 8 hours. In at least one embodiment, the oral dosage forms do not release PRX-3140 for at least 6 hours. In another embodiment, the oral dosage forms do not release PRX-3140 for at least 2 hours.

### VI. Sustained Release Pharmaceutical Compositions

The present disclosure relates to novel sustained release compositions of PRX-3140, and polymorphs and salts thereof, as a compound. The sustained release compositions of the present disclosure provide controlled release of the compound or drug over a longer period of time (e.g., at least 2 hours to about 72 hours, at least 4 hours to 24 hours, at least 6 hours to 48 hours etc.). After about 2 hours post administration or ingestion of a dose, the sustained release compositions of the present disclosure provide prolonged release in the intestines after the passage of the compositions from the stomach to allow for uptake of the compound (PRX-3140 or a polymorph or a salt thereof) from the intestines and/or colon into the blood stream in a subject. The rate of release of PRX-3140, or a polymorph or a salt thereof, from the sustained release compositions (for example enteric-coated delayed release tablet) is slower than that observed for the reference product (the capsule). The sustained release compositions provide a controlled and prolonged exposure of the subject to the compound over a time period from at least about 2 hours to about 270 hours per dose after administration. Thus, by reducing the frequency of dosing and/or reducing the rate of uptake of the compound into blood stream, the present disclosure provides the potential for improved or enhanced tolerance to the drug by the patient and for improved patient convenience and compliance. The slow and prolonged (or sustained) release and uptake into the blood stream of PRX-3140, or a polymorph or a salt thereof, also has the potential to provide reduced local and/or systemic side effects and toxicity and reduced drug accumulation as compared with other dosage forms such as the reference product, and/or reduced total amount of drug for treatment, because the patient may be exposed to lower peak concentration of drug over time. Targeting the intestines (for example, duodenum and jejunum) and colon, by delaying the release of the drug, to protect from the acidic environment of the stomach also increase the potential for improved bioavailability and ability to withstand “food effect” as well as increased efficacy.

The present disclosure provides sustained release compositions comprising PRX-3140, or a polymorph or a salt thereof, and a sustained release delivery system. The sustained release delivery system includes (i) at least one sustained release (SR) or release rate controlling agent (“sustained release agent” or “SR-agent” used interchangeably herein), (ii) at least one binder, and (iii) at least one lubricant.

In some embodiments, the sustained release compositions of the present disclosure are in the form of solid dosage forms such as tablets, mini-tablets, beads, microbeads, granules, spheres particles, multi-particulates, and the like. The present disclosure provides that the sustained release compositions of the present disclosure can be enteric coated for delayed release targeting the intestines and colon. Accordingly, in some embodiments, the sustained release compositions are in the form of enteric coated delayed release tablets, enteric coated delayed release tablet-in-tablets, enteric coated delayed release tablet-in-capsules, beads-in-capsules, spheres-in capsules, and the like.

The PRX-3140 or a polymorph or a salt thereof may be dispersed in the sustained release compositions homogeneously.

In certain embodiments, sustained release compositions may comprise PRX-3140 as at least 0.1%, at least 0.2%, at least 0.3%, at least 0.4%, at least 0.5%, at least 0.6%, at least 0.7%, at least 0.8%, at least 0.9%, at least 1%, at least 5%, at least 10%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.99%, and 100% of PRX-3140 w/w of total PRX-3140 in the sustained release composition. In other embodiments, sustained release compositions comprising PRX-3140 comprise about 0.01% to about 40%, about 0.01% to about 20%, about 0.05% to about 15%, and about 0.1% to about 10% of PRX-3140 w/w of the sustained release composition. In at least one embodiment, the sustained release compositions comprising PRX-3140 comprise 0.01% to 40% of PRX-3140 w/w of the sustained release composition. In various other embodiments, the sustained release compositions comprising PRX-3140 comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, and about 20% of PRX-3140 w/w of the sustained release composition. In some embodiments, the PRX-3140 contained in sustained release compositions is substantially pure. Such sustained release compositions may contain less than 5%, less than 4%, less than 3%, less than 2.5% less than 2%, less than 1%, less than 0.5%, or less than 0.1% PRX-3140. Such sustained release compositions may contain less than 5% less than 4%, less than 3% less than 2%, or less than 1% other impurities.

In certain aspects, the present disclosure provides crystalline forms of PRX-3140, including crystalline forms of PRX-3140 and crystalline forms of mixtures of PRX-3140 and PRX-3140 potassium salt. The present disclosure further provides pharmaceutical compositions of PRX-3140 comprising the crystalline forms described herein. A crystalline form of PRX-3140 may provide the advantage of bioavailability and stability, suitable for use as an active ingredient in a pharmaceutical composition. Variations in the crystal structure of a pharmaceutical drug substance or active ingredient may affect the dissolution rate (which may affect bioavailability, etc.), manufacturability (e.g., ease of handling, ability to consistently prepare doses of known strength) and stability (e.g., thermal stability, shelf life, etc.) of a pharmaceutical drug product or active ingredient. Such variations may affect the preparation or formulation of pharmaceutical compositions in different dosage or delivery forms, such as solid oral dosage forms including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, crystalline forms of PRX-3140 may provide advantages such as: improving the manufacturing process of an active agent or the stability or storability of a drug product form of the compound or an active ingredient, and/or having suitable bioavailability and/or stability as an active agent.

In some embodiments, the sustained release composition comprises PRX-3140 potassium salt predominantly as polymorph Form I. In certain embodiments, a sustained release composition comprises PRX-3140 as at least 0.1%, at least 0.2%, at least 0.3%, at least 0.4%, at least 0.5%, at least 1%, at least 5%, at least 10%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.99%, or 100% of a single polymorphic Form of PRX-3140, such as Form I w/w of total PRX-3140 in the sustained release composition. For example, a sustained release composition may comprise PRX-3140 as Form I as at least 0.1%, at least 0.2%, at least 0.3%, at least 0.4%, at least 0.5%, at least 1%, at least 5%, at least 10%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, at least 99.5%, at least 99.99%, or 100% w/w of the total PRX-3140 in the sustained release composition.

In at least one sustained release composition, the composition comprises ≥90% of a single polymorphic Form of PRX-3140 potassium salt, such as Form I w/w of the total PRX-3140 in the sustained release composition. For example, a sustained release composition comprises ≥90% of Form I of PRX-3140 w/w of the total PRX-3140 in the sustained release composition. In another embodiment, the sustained release composition comprises ≥95% of a single polymorphic Form of PRX-3140, such as Form I, w/w of the total PRX-3140 in the sustained release composition. In yet another embodiment, the sustained release composition comprises ≥96%, ≥97%, ≥98%, ≥99%, or ≥99.5% of a single polymorphic Form of PRX-3140, such as Form I, w/w of the total PRX-3140 in the sustained release composition. For example, the sustained release composition comprises ≥96%, ≥97%, ≥98%, ≥99%, or ≥99.5% of Form I of PRX-3140 w/w of the total PRX-3140 in the sustained release composition. When a particular percentage by weight of PRX-3140 is a single polymorphic form, the remainder of PRX-3140 in the sustained release composition is some combination of amorphous PRX-3140 and/or one or more polymorphic forms of PRX-3140 excluding the single polymorphic form. When the polymorphic PRX-3140 is defined as one particular form of PRX-3140, the remainder is made up of amorphous PRX-3140 and/or one or more polymorphic forms other than the particular form specified. Examples of single polymorphic forms include Forms I of PRX-3140, as well as descriptions of a single polymorphic form characterized by one or more properties as described in Applicant's patent publication U.S. patent application Ser. No. 17/830,519.

In other embodiments, a sustained release composition comprising PRX-3140 comprises about 0.01% to about 20%, about 0.05% to about 15%, or about 0.1% to about 10% of a single polymorphic Form of PRX-3140, such as Form I, w/w of the sustained release composition. In at least one embodiment, the sustained release composition comprising PRX-3140 comprises about 0.01% to about 20% of a single polymorphic Form of PRX-3140, such as Form I, w/w of the composition. In various other embodiments, the sustained release composition comprising PRX-3140 comprises about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 10%, or about 20% of a single polymorphic Form of PRX-3140, such as Form I, wt %/wt % of the sustained release composition. In an aspect, a sustained release composition comprising a single polymorphic Form of PRX-3140, such as Form I, further comprises a second polymorphic Form of PRX-3140.

In an aspect, a sustained release composition comprising a polymorphic Form of PRX-3140 in amounts ranging from about 0.1 mg to about 200 mg in a unit dose. In various embodiments, the sustained release compositions comprise 0.1 mg, 0.5 mg, 1 mg, 2 mg, 3 mg, 4 mg, 5 mg, 6 mg, 7 mg, 8 mg, 9 mg, 10 mg, 20 mg, 50 mg, 75 mg, 100 mg, 200 mg and 250 mg of a polymorphic Form of PRX-3140 such as Form I, or a combination thereof. For example, a sustained release composition may comprise 0.1 mg, 0.5 mg, 1 mg, 2 mg, 3 mg, 4 mg, 5 mg, 6 mg, 7 mg, 8 mg, 9 mg, 10 mg, 20 mg, 50 mg, 75 mg, 100 mg, 200 mg and 250 mg of Form I of PRX-3140. In some embodiments, a sustained release composition may comprise from 0.1 mg to 1 mg, from 0.1 mg to 2 mg, from 0.1 mg to 3 mg, from 0.1 mg to 4 mg, from 0.1 mg to 5 mg, from 1 mg to 2 mg, from 1 mg to 3 mg, from 1 mg to 4 mg, from 1 mg to 5 mg, from 1 mg to 6 mg, from 1 mg to 7 mg, from 1 mg to 8 mg, from 1 mg to 9 mg, from 1 mg to 10 mg, from 5 mg to 6 mg, from 5 mg to 7 mg, from 5 mg to 8 mg, from 5 mg to 9 mg, from 5 mg to 10 mg, from 5 mg to 15 mg, from 5 mg to 20 mg, from 5 mg to 40 mg, from 10 mg to 15 mg, from 10 mg to 20 mg, or from 10 mg to 40 mg a polymorphic Form of PRX-3140 such as Form I.

While the content of PRX-3140 or a polymorph or a salt thereof in the sustained release compositions of the present disclosure varies depending on the dosage form of the sustained-release composition, target disease, severity of disease, and the like, it is an amount generally corresponding or equivalent to from about 0.01 mg to about 200 mg of PRX-3140. One of skill in the art will recognize that when sustained release compositions include salts of PRX-3140, the PRX-3140 salt will be in an equivalent amount on the basis of PRX-3140 to be released.

In an aspect, the present disclosure provides that the sustained release compositions of the present disclosure show a sustained release of the drug PRX-3140, or a polymorph or a salt thereof, from the sustained release composition over a period of about 2 hours to about 72 hours. In some embodiments, the sustained release compositions of the present disclosure show a sustained release of the drug PRX-3140, or a polymorph or a salt thereof, from the sustained release composition over a period of about 4 hours to about 24 hours or more. In some embodiments, the sustained release compositions of the present disclosure release PRX-3140, or a polymorph or a salt thereof, in a sustained manner over a period of at least 2 hours, at least 3 hours at least 4 hours, at least 5 hours, at least 6 hours, at least 7 hours, at least 8 hours, at least 9 hours, at least 10 hours, at least 12 hours, at least 14 hours, at least 16 hours, at least 18 hours, at least 24 hours, at least 48 hours, and at least 72 hours. In some embodiments, the sustained release PRX-3140 or a polymorph or a salt thereof is released over a period of from 6 hours to 48 hours. In some embodiments, the PRX-3140 or a polymorph or a salt thereof is released from the sustained release compositions after about 2 hours pose dose for a period ranging from about 2 hours to about 72 hours. In some embodiments, after about 2 hours post dose the PRX-3140 or a polymorph or a salt thereof is released from the sustained release compositions for about 2 hours, about 3 hours, about 4 hours, about 5 hours, about 6 hours, about 7 hours, about 8 hours, about 9 hours, about 10 hours, about 11 hours, about 12 hours about 13 hours, about 14 hours, about 15 hours, about 16 hours, about 17 hours, about 18 hours, about 19 hours, about 20 hours, about 21 hours, about 22 hours, about 23 hours and about 24 hours. In some embodiments, the rate of release of the compound (PRX-3140 or a polymorph or a salt thereof) is slower and more sustained or prolonged than that of a reference product, the enteric resistant delayed release PRX-3140 capsule (“Capsule” or “Control” as used interchangeably herein) as shown herein this disclosure.

In dissolution tests of certain sustained release compositions of the present disclosure conducted according to USP I Method, the sustained release compositions show percentage dissolution ranging from about 0% to 35% at 3 hours, from about 35% to about 55% at 12 hours, and from about 65% to 85% at 24 hours in a dissolution test according to the 50 RPM USP I method and using water or pH 1.2 at 37 degrees Celsius for 2 hours in simulated gastric fluid and pH 6.8 at 37 degrees Celsius for 24 hours in simulated intestinal fluid as a test medium.

A method for measuring a dissolution of a composition (e.g., a sustained release composition) may comprise measuring the dissolution in water or an acid phase (e.g., simulated gastric fluid) followed by a buffer phase (e.g., simulated intestinal fluid). The acid phase may comprise placing 750 mL of 0.1 N hydrochloric acid in the vessel, and assembling the apparatus (e.g., the paddle apparatus or the basket apparatus). The medium may be allowed to equilibrate to a temperature of 37+/−0.5 degrees Celsius. One dosage unit may be placed in the apparatus, the vessel covered, and the apparatus operated at the specified rate. After 2 hours of operation in 0.1 N hydrochloric acid, an aliquot of the fluid may be withdrawn. Then the method may proceed immediately as directed under the buffer stage. An analysis of the aliquot may be performed using a suitable assay method. While operating the apparatus at a specified rate, 250 mL of 0.20 M tribasic sodium phosphate that has been equilibrated to 37+/−0.5 degrees Celsius may be added to the fluid in the vessel. The fluid may be adjusted, if necessary, with 2N hydrochloric acid or 2 N sodium hydroxide to a pH of 6.8+/−0.05. The apparatus may continue to be operated for 45 minutes, or for the specified time. At the end of the time period, an aliquot of fluid may be withdrawn, and an analysis of the aliquot may be performed using a suitable assay method.

In some embodiments, the percentage dissolution is measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2. The percentage dissolution refers to the percentage of the composition that has dissolved, by weight percent. The percentage dissolution refers to the weight percent dissolution of PRX-3140, or the polymorph or salt thereof, during the dissolution study. As a non-limiting example, if a tablet comprises 4 mg of PRX-3140, and during the dissolution study at a time point of 3 hours, 1 mg of the PRX-3140 is dissolved into the solution, then the percentage dissolution at 3 hours is 25%. The percentage dissolution can be determined by, for example, high pressure liquid chromatography.

In some embodiments, the sustained release composition has a percentage dissolution ranging from about 5% to about 35%, from about 10% to about 15%, from about 20% to about 25%, from about 30% to about 35%, from about 5% to about 30%, from about 5% to about 25%, from about 5% to about 20%, from about 5% to about 15%, from about 5% to about 10%, from about 10% to about 15%, from about 15% to about 20%, from about 20% to about 25%, from about 25% to about 30%, or from about 30% to about 35% at 3 hours, measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In some embodiments, the sustained release composition has a percentage dissolution at 2 hours of no more than about 5%, no more than about 10%, no more than about 15%, no more than about 20%, no more than about 25%, no more than about 30%, no more than about 35%, no more than about 40%, no more than about 45%, or no more than about 50%, measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2. In some embodiments, the sustained release composition has a percentage dissolution of not more than (NMT) about 30%, NMT about 25%, NMT about 20%, NMT about 15%, NMT about 10%, or NMT about 5% at 3 hours.

In some embodiments, the sustained release composition has a percentage dissolution of PRX-3140 or a polymorph or a salt thereof at 2 hours of less than about 5% and at 3 hours ranging from about 5% to about 80%, from about 10% to about 75%, from about 20% to about 70%, from about 30% to about 65%, from about 5% to about 75%, from about 5% to about 70%, from about 5% to about 60%, from about 5% to about 65%, from about 5% to about 60%, from about 10% to about 55%, from about 5% to about 50%, from about 30% to about 80%, from about 25% to about 75%, from about 35% to about 80%, or not less than about 70%, as measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In some embodiments, the sustained release composition has a percentage dissolution of PRX-3140 or a polymorph or a salt thereof at 7 hours of less than about 10%, less than about 15%, less than about 20%, less than about 25%, less than about 30%, less than about 31%, less than about 32%, less than about 33%, less than about 34%, less than about 35%, less than about 36%, less than about 37%, less than about 38%, less than about 39%, less than about 40%, as measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In some embodiments, the sustained release composition has a percentage dissolution of PRX-3140 or a polymorph or a salt thereof at 2 hours of less than about 5% and at 6 hours ranging from about 70% to about 99%, from about 75% to about 95%, from about 85% to about 95%, from about 90% to about 95%, from about 75% to about 90%, from about 85% to about 90%, from about 75% to about 85%, from about 80% to about 99%, from about 85% to about 99%, from about 90% to about 99%, from about 95% to about 99% or not less than about 95%, as measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In some embodiments, the sustained release composition has a percentage dissolution of PRX-3140 or a polymorph or a salt thereof at 2 hours of less than about 5% and at 6 hours of not less (NLT) than about 20%, about 25%, about 30%, about 35%, or about 40%. In some embodiments, the sustained release composition has a percentage dissolution of PRX-3140 or a polymorph or a salt thereof at 2 hours of less than about 5% and at 9 hours of NLT about 40%, about 45%, about 50%, about 55%, or about 60%. In some embodiments, the sustained release composition has a percentage dissolution ranging from about 35% to about 55%, from about 40% to about 55%, from about 45% to about 55%, from about 50% to about 55%, from about 35% to about 50%, from about 35% to about 45%, from about 35% to about 40%, from about 35% to about 40%, from about 40% to about 45%, from about 45% to about 50%, or from about 50% to about 55% at 12 hours, measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In some embodiments, the sustained release composition has a percentage dissolution of PRX-3140 or a polymorph or a salt thereof of less than 20% at 2 hours, and a percentage dissolution of at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% at 12 hours, measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In some embodiments, the sustained release composition has a percentage dissolution of PRX-3140 or a polymorph or a salt thereof at 2 hours of less than about 5% and at 72 hours ranging from about 55% to about 100%, from about 60% to about 99%, from about 75% to about 90%, from about 80% to about 85%, from about 55% to about 99%, from about 65% to about 99%, from about 70% to about 99%, from about 75% to about 99%, from about 80% to about 99%, from about 90% to about 99%, from about 80% to about 95%, or from about 75% to about 95%, as measured by the 50 RPM USP paddle method in water or simulated gastric fluid at pH 1.2 and at 37 degrees Celsius from hours 0-2, and in simulated intestinal fluid at pH 6.8 and at 37 degrees Celsius after hour 2.

In an aspect, the sustained release compositions of the present disclosure are formulated or prepared as solid dosage forms including, but not limited to, tablets, mini-tablets, caplets, beads, microbeads, spheres, pellets, microspheres, granules, pills, tablet-in-tablet, tablets-in-capsule, granules in capsules, and the like. The sustained release compositions of the present disclosure in the form of solid dosage form provide slow and controlled release of the drug, the active ingredient PRX-3140 or a polymorph or a salt thereof over a surprisingly longer period of time than that observed with other compositions containing PRX-3140 (e.g., at least about 4 hours to about 24 hours, at least about 2 hours to about 72 hours). In at least one embodiment, the sustained release composition in the form of a solid dosage form is a tablet. Such sustained release compositions may be coated or uncoated. In an aspect, the present disclosure provides that the solid dosage forms of the present disclosure are capable of being delivered orally.

In an aspect, the present disclosure provides that the active ingredient (PRX-3140 or a polymorph or a salt thereof) is dispersed in a sustained release delivery system. PRX-3140 or a salt or polymorph thereof may be evenly or homogeneously dispersed. The sustained release delivery system includes but is not limited to, at least one controlled release agent such as a sustained release agent that controls the release rate of the drug PRX-3140 or a salt or polymorph thereof, at least one binder, and at least one lubricant. In some embodiments, the sustained release delivery system is present in the sustained release compositions of the present disclosure in an amount ranging from about 1% to about 99.99%.

In some embodiments, the sustained release delivery system is present in the sustained release compositions of the present disclosure in an amount ranging from about 0.1 mg to about 499.5 mg; from about 25 mg to about 450 mg; from about 50 to about 400 mg, or from about 100 mg to about 300 mg. In at least one embodiment, the sustained release delivery system is present in the sustained release compositions in an amount from about 50 mg to about 199.5 mg. In another embodiment, the sustained release delivery system is present in the sustained release compositions in an amount of about 30 mg, about 40 mg, about 50 mg, about 60 mg, about 70 mg, about 80 mg, about 90 mg, about 95 mg, or about 100 mg. In still another embodiment, the sustained release delivery system is present in the sustained release compositions in an amount of about 110 mg, about 120 mg, about 125 mg, about 130 mg, about 140 mg, about 145 mg, about 150 mg, about 160 mg, about 170 mg, about 180 mg, about 190 mg, about 195 mg, about 200 mg, about 210 mg, about 225 mg, about mg, about 300 mg, about 400 mg, about 450 mg, about 460 mg, about 470 mg, about 480 mg, about 490 mg, about 495 mg or about 499.5 mg. In some embodiments, the sustained release delivery system is present in the sustained release compositions in an amount from about 5 mg to 99.5 mg.

In some embodiments, the sustained release compositions of the present disclosure, the ratio of PRX-3140 or a polymorph or a salt thereof to the sustained release delivery system is generally about 4:1 to about 0.1:100. In some embodiments, the ratio of PRX-3140 or a polymorph or a salt thereof to the sustained release delivery system is 2.5:1 to about 1:20.

Sustained release agent present in a sustained release composition of the present disclosure may be any sustained release agent known in the art to slow the release of a hydrophobic drug such as PRX-3140 or a polymorph or a salt thereof.

Examples of sustained release agents include cellulosic ethers, gums, acrylic resins such as polymers and copolymers of acrylic acid, methacrylic acid, methyl acrylate, methyl methylacrylate, and combinations thereof, polyvinyl pyrrolidine, and protein-derived compounds. Examples of cellulosic ethers include hydroxyalkyl celluloses, hydroxyethyl celluloses, hydroxypropyl celluloses, hydroxypropylmethyl celluloses (HPMC or hypromellose, for example Nos. 2208, 2906, 2910), carboxyalkyl celluloses, and carboxymethyl celluloses. In some embodiments, the at least one sustained release agent is a pH sustained release agent such as acid insoluble polymers which become increasingly soluble and permeable above pH 5.0 but remaining impermeable below pH 5.0. Such controlled release polymers target upper small intestines and/or colon. Non-limiting examples of acid-insoluble polymers include cellulose acetate phthalate, cellulose acetate butyrate, hydroxypropyl methyl cellulose phthalate, algenic acid salts such as sodium or potassium alginate, shellac, polyvinyl acetate/polyvinyl pyrrolidone, xanthan gum, acrylic acid-methylacrylic acid copolymers, including those available commercially from Evonik or Rohm ((Eudragit® sustained release polymers Eudragit® RL (high permeability), Eudragit® RS (low permeability) and Eudragit® NM 30D (low permeability), alone or in any combination thereof to achieve the desired permeability for sustained release. The viscosity of sustained release agents may be any viscosity suitable for sustained release of PRX-3140 or a polymorph or a salt thereof. In certain embodiments, the viscosity of the at least sustained release agent ranges from about 1000 mPa·s to about 150,000 mPa·s. In some embodiments, the sustained release delivery system includes one or more SR/release rate controlling agents with viscosity ranging from about 1000 mPa·s to about 10,000 mPa·s, from about 10,000 mPa·s to about 70,000 mPa·s, from about 70,000 mPa·s to about 150,000 mPa·s. or a combination thereof. In some embodiments, the present disclosure provides that the sustained release delivery system includes two or more sustained release agents. Each sustained release agent may have the same viscosity or a differing viscosity, for example one sustained release agent may have a viscosity ranging from about 1000 mPa·s to about 10,000 mPa·s, while other sustained release agent may have a viscosity of about 10,000 mPa·s to about 70,000 mPa·s or about 70,000 mPa·s to about 150,000 mPa·s.

In some embodiments, the sustained release agent is HPMC/hypromellose (e.g., Nos. 2208, 2906, 2910). Hypromellose to be used in the present disclosure has a number average weight molecular (Mn) of about 86,000 to 220,000. In some embodiments, hypromellose has a number average weight molecular (Mn) of generally 10,000 to 250,000 or greater. Hypromellose is commercially available from Dow Chemicals under the trade name Methocel™, for example, Methocel™ E4M (Mn 86,000, 2% in water viscosity ~4,000 mPa·s). Methocel™ K100 (Mn=220,000, 2% viscosity; ~100,000 mPa·s); Methocel™ K15M (Mn=120,000, 2% viscosity; ~15,000 mPa·s); Methocel™ K4M (Mn=86,000, 2% viscosity; ~4,000 mPa·s). Hypromellose of one grade may be used alone or in combination with another grade.

When a sustained release composition showing release of PRX-3140 or a polymorph or a salt thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16, at least 18 hours, at least 24 hours, at least 48 hours, and at least 72 hours is obtained, in some embodiments the sustained release agent, such as Hypromellose, has an number average molecular weight generally ranging from about 86,000 to about 220,000 daltons. In at least one embodiment, the number average molecular weight of about 220,000 daltons.

The amount of sustained release agent in the composition may be any amount effective to delay the release of the compound PRX-3140, or a polymorph or a salt thereof, for about 2 hours post-dose to protect the compound from the acidic environment of the stomach and allow passage of the compound through the stomach into the intestines and prolong such release for a period of about 2 hours to about 72 hours. The amount of sustained release agent in the composition may be any amount effective to provide a slower rate of release of the compound PRX-3140, or a polymorph or a salt thereof as compared with the reference product. In some embodiments, the amount of sustained release agent in the composition may be any amount effective to delay the release of the compound PRX-3140, or a polymorph or a salt thereof, for at least about 1 hour, at least about 1.1 hours, at least about 1.2 hours, at least about 1.3 hours, at least about 1.4 hours, at least about 1.5 hours, at least about 1.6 hours, at least about 1.7 hours, at least about 1.8 hours, at least about 1.9 hours, at least about 2 hours, at least about 2.1 hours, at least about 2.2 hours, at least about 2.3 hours, at least about 2.4 hours, or at least about 2.5 hours post-dose, as compared with the reference product.

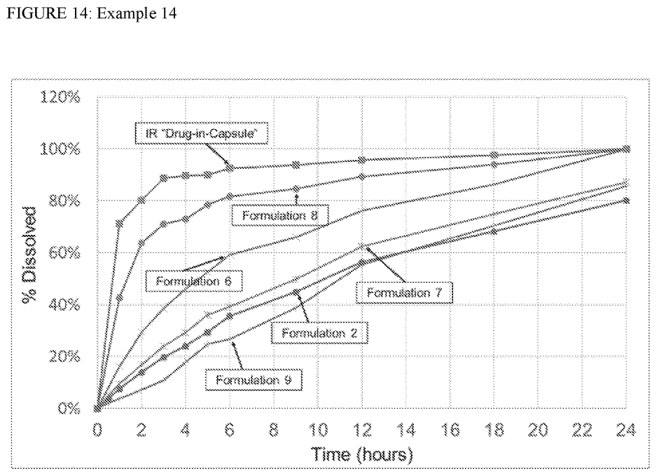

When a sustained release composition shows percentage dissolution ranging from about 0% to 35% at 3 hours, from about 35% to about 55% at 12 hours, and from about 65% to 85% at 24 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium, at least one sustained release agent, such as Hypromellose (HPMC), may generally be present in a sustained release composition of the present disclosure in amounts from about 0.1% to about 99%, from about 0.1% to about 90%, from about 5% to about 90%, from about 5% to about 80%, from about 5% to about 70%, and from about 5% to about 60% w/w of the sustained release composition. In some embodiments, the sustained release agent (e.g., a gum, an acrylic resin, methacrylic acid, methyl acrylate, methyl methylacrylate, polyvinyl pyrrolidine, a protein-derived compound, a hydroxyalkyl cellulose, a hydroxyethyl cellulose, a hydroxypropyl cellulose, a hydroxypropylmethyl celluloses, a carboxyalkyl cellulose, or, a carboxymethyl cellulose) may be present in an amount of from about 10% to about 40%, from about 10% to about 50%, from about 10% to about 60%, from about 20% to about 40%, from about 20% to about 50%, from about 20% to about 60%. In some embodiments, the sustained release agent may be present in an amount of at least about 10%, at least about 20%, at least about 30%, or at least about 40%. Such sustained release compositions of the present disclosure as disclosed herein release PRX-3140 or polymorphs or salts thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16 hours, at least 18 hours, at least 24 hours, at least 48 hours, and at least 72 hours. See Example 14 and FIG. 14. In some embodiments, the compound in the sustained release composition is released over a period of from 6 hours to 48 hours. In some embodiments, the compound (e.g., PRX-3140 or a polymorph or a salt thereof) is released over a period of from 2 hours to 72 hours as tested by USP I method and using water or pH 1.2 at 37 degrees Celsius for 2 hours in simulated gastric fluid and pH 6.8 at 37 degrees Celsius for 24 hours in simulated intestinal fluid.

As non-limiting examples, the ratio of PRX-3140 or a polymorph or a salt thereof to sustained release agent ranges from about 1:20 to about 20:1, from about 1:10 to about 10:1, from about 1:5 to about 2:5 to prepare sustained release compositions of the present disclosure. To prepare sustained release tablets containing about 0.1 mg to about 200 mg PRX-3140 or a salt thereof, drug to sustained release agent will range from about 20:1 to about 1:20. In some embodiments drug to sustained release agent in sustained release tablets containing about 0.1 mg to about 200 mg PRX-3140 or a salt thereof will range from about 10:1 to about 1:10. In certain embodiments, the ratio of the therapeutic drug PRX-3140 or a polymorph or a salt thereof to the sustained release agent is about 1:5. One of skill will recognize that the rate of release of PRX-3140 or a polymorph or a salt thereof may be adjusted for sustained release as desired by varying the viscosity grade of the sustained release compositions of the present disclosure and the amount of sustained release agent as disclosed herein.

Binder present in a sustained release composition of the present disclosure may be any binder known in the art that can hold the components or the ingredients in the sustained release compositions of the present disclosure together. Binders suitable for use in the sustained release compositions provided herein include, but are not limited to, monosaccharides (such as sucrose, dextrose, fructose), disaccharides, starches such as corn starch, potato starch, or starches such as starch paste, pregelatinized starch, and starch 1500, polyhedric alcohols, mannitol, xylitol, sorbitol, lactose, polyethylene glycols such as PEG 6000, natural and synthetic gums such as acacia, sodium alginate, alginic acid, other alginates, tragacanth, guar gum, cellulose (e.g., powdered cellulose and pregelatinized starch) and its derivatives (e.g., ethyl cellulose, cellulose acetate, methyl cellulose, carboxymethyl cellulose calcium, sodium carboxymethyl cellulose), polyvinyl pyrrolidone, methyl cellulose, polyvinyl pyrrolidone, hydroxypropyl methyl cellulose (e.g., Nos. 2208, 2906, 2910), crystalline cellulose, microcrystalline cellulose (MCC), silicified microcrystalline cellulose, calcium carbonate and salts thereof, calcium phosphate, precipitated calcium phosphate, sodium carbonate, sodium phosphate, anhydrous dibasic calcium phosphate, talc, dextrates, kaolin, mannitol, silicic acid, sorbitol and combinations or mixtures thereof. Suitable forms of crystalline and microcrystalline cellulose include, but are not limited to, the materials commercially sold as AVICEL® PH 101, AVICEL PH 103 AVICEL® RC 581, AVICEL® PH 105 (available from FMC Corporation, American Viscose Division, Avicel Sales, Marcus Hook, Pa.), Pharmacel 101. Anhydrous or low moisture excipients are preferable. Suitable anhydrous or low moisture excipients or additives include microcrystalline cellulose commercially available as AVICEL® PH 101, AVICEL® PH 103, Pharmacel 101. In at least one embodiment, the binder is microcrystalline cellulose (such as AVICEL® PH 101) or silicified microcrystalline cellulose.

The sustained release compositions of the present disclosure typically may comprise at least one binder in amounts ranging from about 1% to about 99%, from about 5% to about 95%, from about 10% to about 90%, from about 15% to about 85%, from about 20% to about 80%, or from about 20% to about 85% on a w/w basis relative to the weight of the sustained release composition. In certain embodiments, the sustained release compositions of the present disclosure typically comprise microcrystalline cellulose in amounts ranging from about 1% to about 99%, from about 5% to about 95%, from about 10% to about 90%, from about 15% to about 85%, from about 20% to about 80%, or from about 20% to about 85% on a w/w basis relative to the weight of the sustained release composition.

Exemplary lubricants that can be used in the sustained release compositions provided herein include, but are not limited to, stearic acid, calcium stearate, magnesium stearate, zinc stearate, potassium stearate, hydrogenated vegetable oil (e.g., peanut oil, cottonseed oil, sunflower oil, sesame oil, olive oil, corn oil, and soybean oil), mineral oil, light mineral oil, glycerin, sorbitol, mannitol, glycols, polyethylene glycol, sodium lauryl sulfate, magnesium lauryl sulfate, macrogol, talc, ethyl oleate, ethyl laureate, agar, waxes, and combinations thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), Q7-9120 (Dow Corning), and combinations thereof. In some embodiments, the lubricant is magnesium stearate. Magnesium stearate reduces the friction between the die wall and tablet mix during the compression and ejection of the tablets. It helps prevent adhesion of tablets to the punches and dies. Magnesium stearate also aids in the flow of the powder in the hopper and into the die. It has a particle size range of 450-550 microns and a density range of 1.00-1.80 g/mL It is stable and does not polymerize within the tableting mix. One lubricant, magnesium stearate, may also be employed in the formulation.

Typically, a sustained release composition of the present disclosure may include at least one lubricant in an amount ranging from about 0.01% to about 5%, from about 0.2% to about 2%, or from about 0.5% to about 1.5% on a w/w basis relative to the weight of the sustained release composition. In certain embodiments, a sustained release composition of the present disclosure includes magnesium stearate in an amount ranging from about 0.01% to about 5%, from about 0.2% to about 2%, or from about 0.5% to about 1.5% on a w/w basis relative to the weight of the sustained release composition.

Certain exemplary embodiments of sustained release compositions of the present disclosure include, but are not limited to, the following embodiments.

In some embodiments, at least one sustained release agent (e.g., HPMC, cellulose acetate phthalate, cellulose acetate butyrate, hydroxypropyl methyl cellulose phthalate, sodium alginate, potassium alginate, shellac, polyvinyl acetate/polyvinyl pyrrolidone, xanthan gum, or acrylic acid-methylacrylic acid copolymers) is present in a sustained release composition in an amount ranging from about 0.1% to about 99%; at least one binder is present in a sustained release composition in an amount ranging from about 1% to about 99%; the at least one lubricant is present in the sustained release delivery system in an amount ranging from about 0.01% to about 5% wherein the percentages are on a w/w basis relative to the weight of the sustained release composition.

In some embodiments, at least one sustained release agent is present in a sustained release composition in an amount ranging from about 0.1% to about 90%; at least one binder is present in a sustained release composition in an amount ranging from about 1% to about 99%; the at least one lubricant is present in the sustained release delivery system in an amount ranging from about 0.01% to about 5% wherein the percentages are on a w/w basis relative to the weight of the sustained release composition.

In at least one embodiment, the sustained release composition includes the at least one sustained release agent in an amount ranging from about 5% to 60%; the at least one binder in an amount ranging from about 10% to about 90%, and the at least one lubricant in an amount ranging from about 0.01% to about 5% wherein the percentages are on a w/w basis relative to the weight of the sustained release composition.

In some embodiments, at least one sustained release agent is present in a sustained release composition in an amount ranging from about 0.1% to about 10%; at least one binder is present in a sustained release composition in an amount ranging from about 80% to about 99%; the at least one lubricant is present in the sustained release delivery system in an amount ranging from about 0.02% to about 2% wherein the percentages are on a w/w basis relative to the weight of the sustained release composition.

In at least one embodiment, the sustained release composition includes the at least one sustained release agent in an amount ranging from about 10% to 45%; the at least one binder in an amount ranging from about 50% to about 80%, the at least one lubricant in amounts ranging from about 0.02% to about 2% wherein the percentages are on a w/w basis relative to the weight of the sustained release composition.

In certain embodiments, the at least one sustained release agent is present in the sustained release composition in an amount of about 8%, about 9%, about 10%, about 12%, about 15%, about 18%, about 20%, about 22% about 25%, about 30%, about 35%, about 40%, about 45%, about 50% about 55%, about 60%; the at least one binder is present in the sustained release composition in an amount of about 30%, about 35%, about 40%, about 45%, about 50% about 55%, about 60%, about 65%, about 70%, about 75%, or about 80%; and the at least one lubricant of about 0.02%, about 0.05%, about 0.1%, about 1.2%, about 1.3%, about 1.4%, about 1.5%, about 1.6%, about 1.7%, about 1.8%, about 1.9% or about 2%, wherein the percentages are on a w/w basis relative to the weight of the sustained release composition.

In certain embodiments, the at least one sustained release agent is present in the sustained release composition in an amount of about 15%, about 16%, about 17%, about 18%, about 19%, about 20%, about 21%, about 22%, about 23%, about 24%, or about 25%; at least one binder is present in the sustained release composition in an amount of about 29%, about 25%, about 30%, about 35%, about 39%, about 40%, about 45%, about 49%, about 50%, about 55%, about 60%, about 64%, about 69%, about 70%, about 71%, about 72%, about 73%, about 74%, about 75%, about 76%, about 77%, about 78%, about 79% or about 80%; and at least one lubricant is present in the sustained release composition in an amount of about 0.8%, about 0.9%, about 1%, about 1.2%, about 1.3%, about 1.4%, or about 1.5%, in each case on a w/w basis relative to the weight of the sustained release composition.

In at least one embodiment, the at least one sustained release agent is present in the sustained release composition in an amount of about 10%, about 20%, or about 40; at least one binder is present in the sustained release composition in an amount of about 55%, about 75%, about 80%, about 85% or about 87%; and at least one lubricant is present in the sustained release composition in an amount of about 0.9% to about 1.1%. The percentages are on a w/w basis relative to the weight of the sustained release composition.

In certain embodiments, the sustained release compositions of the present disclosure include additional sustained release agents, binders, lubricants or any combination thereof.

The compositions of the present disclosure and the sustained release delivery systems may further include a pharmaceutically acceptable excipient. The pharmaceutically acceptable excipient may be any additive conventionally used in the technical field of preparation such as fillers, stabilizers, glidants, surfactants, light shielding agents, sweeteners, colorants, flavorants, anti-oxidants, preservatives, reducing agent, chelating agent and the like. The pharmaceutically acceptable excipients are used in an amount conventionally used in the technical field of preparation. In addition, two or more kinds of these additives may be mixed at an appropriate ratio and used.

Examples of fillers suitable for use in the sustained release compositions of the present disclosure include, but are not limited to, talc, calcium carbonate (e.g., granules or powder), sugars such as dextrose, sucrose, lactose, a salt such as calcium carbonate, calcium phosphate, sodium carbonate, sodium phosphate, starches, microcrystalline cellulose, powdered cellulose, cellulosic bases such as methyl cellulose, carboxymethyl cellulose, dextrates, kaolin, mannitol, silicic acid, sorbitol, starch, pregelatinized starch, and mixtures thereof.

Examples of stabilizers include sodium ascorbate tocopherol, tertrasodium edetate, cyclodextrins, nicotinic acid amide, alkaline earth metal salts (e.g., calcium carbonate, calcium hydroxide, magnesium carbonate, magnesium silicate magnesium aluminate) and butylhydroxyanisole.

Examples of glidants include, but are not limited to, colloidal silicone dioxide, cellulose, calcium phosphate, di or tri-basic and the like. Examples of surfactants include sodium lauryl sulfate, polysorbate 80, and the like. Examples of light shielding agents include titanium oxide. Light shielding agents are desirable for light sensitive active ingredients such as PRX-3140, and polymorphs and salts thereof. Examples of anti-oxidants include butylhydroxytoluene (BHT), butylhydrozyanysole (BHA), tocopherol, tocopherol esters (e.g. tocopherol acetate), ascorbic acid or alkali or alkaline earth metal salt thereof, lycopene and beta-carotene. Examples of reducing agents include cystine and cysteine. Examples of chelating agents include EDTA or alkali metal or alkaline earth metal salt thereof. Examples of colorants include food colors such as Food Color Yellow No. 5, US Food color No. 6, Swedish orange, Food color Blue No. 2, Food color Red No. 2 and the like, food lake colors, yellow ferric oxide, (yellow ferric oxide pigment), red ferric oxide (red ferric oxide pigment), black ferric oxide (black ferric oxide pigment), riboflavin, riboflavin organic acid ester, riboflavin phosphate or alkali metal or alkaline metal earth metal slats thereof, phenolphthalein, titanium oxide, lycopene, and beta-carotene. Examples of sweeteners include aspartame, acesulfame potassium, thaumatin, saccharin sodium and dipotassium glycyrrhizinate. Examples of flavorants include menthol, peppermint oil, lemon oil, vanillin, and strawberry.

The shape of the sustained release compositions in the form of solid dosage forms, such as a tablet, is not particularly limited, and may be any shape suitable for administration, for example, for oral administration, such as spherical, oval, ellipsoidal, pear, cylindrical, cubic, regular and/or irregular shaped. The tablet may have one of a variety of different shapes. For example, the tablet may be shaped as a polyhedron, such as a cube, pyramid, prism, or the like; or may have the geometry of a space figure with some non-flat faces, such as a cone, truncated cone, cylinder, sphere, torus, or the like. In certain embodiments, a tablet has one or more major faces. For example, the tablet surface typically has opposing upper and lower faces formed by contact with the upper and lower punch faces in the compression machine. In such embodiments the tablet surface typically further includes a “belly-band” located between the upper and lower faces, and formed by contact with the die walls in the compression machine.

In an aspect, the present disclosure provides that the sustained release compositions in the form of sustained release tablets have a tablet weight of about 60 to about 500 mg. In some embodiments, sustained release compositions in the form of sustained release tablets have a tablet weight of about 90 to about 110 mg. The sustained release compositions in the form of sustained release tablets have a tablet thickness of about 2.75 to about 3.75 mm. In some embodiments, the sustained release composition in the form of sustained release tablets have a tablet thickness of about 3 to about 3.5 mm.

The hardness of the tablets of the present invention may vary, depending on a variety of factors, including, for example, the relative amounts and specific types of ingredients used, the tableting equipment employed, and the selected processing parameters. The compaction and pressure used to prepare the tablets can influence the release profile of the compound PRX-3140 or a polymorph or a salt thereof into the subject. The compaction and pressure used to prepare the tablets of the present invention may vary depending upon their surface area and the amount and particle size of the compound PRX-3140, or a polymorph or a salt thereof, sustained release agents, binders, lubricants, and other excipients included in the tablet. The degree of hydration and solvation of the components in the composition will also be important in determining the hardness of the tablets. In some embodiments, the sustained release composition in the form of sustained release tablets have a tablet hardness of about 4 Kilopond (Kp) to about 16 Kp. In some embodiments, the sustained release composition in the form of sustained release tablets about 10 to about 16 Kp. In some embodiments, the sustained release composition in the form of sustained release tablets have a tablet hardness of about 4 Kp, about 4.5 Kp, about 5 Kp, about 5.5 Kp, about 6 Kp, about 6.5 Kp, about 7 Kp, about 7.5 Kp, about 8 Kp, about 8.5 Kp, about 9 Kp, about 9.5 Kp, about 10 Kp, about 10.5 Kp, about 11 Kp, about 11.5 Kp, about 12 Kp, about 12.5 Kp, about 13 Kp, about 13.5 Kp, about 14 Kp, about 14.5 Kp, about 15 Kp, about 15.5 Kp and about 16 Kp. In some embodiments, the sustained release composition in the form of sustained release tablets have a tablet hardness of about 13 Kp. In at least one embodiment, the sustained release composition in the form of sustained release tablets have a tablet hardness of 5 Kp.

The friability of the sustained release tablets is typically not more than (NMT) 1.0%. In some embodiments, the sustained release compositions in the form of sustained release tablets include a light shielding agent such as titanium oxide. The sustained release compositions in the form of sustained release tablets can be of any color known in the art, for example Food Color Yellow No. 5, US Food color No. 6, Swedish orange, Food color Blue No. 2, Food color Red No. 2 and the like, food lake colors, yellow ferric oxide, (yellow ferric oxide pigment), red ferric oxide (red ferric oxide pigment), black ferric oxide (black ferric oxide pigment), riboflavin, riboflavin organic acid ester, riboflavin phosphate or alkali metal or alkaline metal earth metal slats thereof, phenolphthalein, titanium oxide, lycopene, and beta-carotene. In some embodiments, the sustained release compositions in the form of sustained release tablets are white or off-white in color. The sustained release compositions, such as the sustained release tablets described herein, may be coated or uncoated.

In another aspect, the present disclosure provides that the sustained release compositions of the present disclosure are used to prepare enteric coated delayed release solid dosage forms such as enteric coated delayed release tablets. An advantageous aspect of such enteric coated compositions is that these sustained release compositions, including enteric coated delayed release tablets, are formulated to minimize the release of PRX-3140 or a polymorph or a salt thereof in stomach to avoid destruction of PRX-3140 or a polymorph or a salt thereof, and to target the release of the active ingredient PRX-3140 or a polymorph or a salt thereof into the small intestine or colon or both. Targeting the intestines and colon increases the bioavailability of the PRX-3140 or a polymorph or salt thereof. In some embodiments, the sustained release compositions of the present disclosure deliver PRX-3140 or a polymorph or a salt thereof into duodenum or jejunum.

One of skill in the art will recognize that the sustained release compositions of the present disclosure function as sustained release compositions in themselves (See Examples 10 to 17), and they can also serve as “cores” (e.g., core tablets) used in the preparation of enteric coated dosage forms (such as enteric coated delayed release tablets or enteric coated delayed release capsules) that provide delayed release of the PRX-3140 or a polymorph or a salt thereof targeting small intestines or colon or both.

Accordingly, in some embodiments the sustained release compositions of the present disclosure in a solid dosage form (for example, as a tablet) forms a “core” composition (e.g., core tablet) for the preparation of an enteric coated delayed release composition. Such sustained release compositions allow for the protection of the acid sensitive active ingredient PRX-3140 or a polymorph or a salt thereof from the acidic environment of the stomach and dissolve at pH higher than 5.5., generally in the intestines, for example in duodenum, jejunum and/or ileum of the small intestine and/or in colon. The enteric coating protects the interconversion of active PRX-3140 to PRX-3140 in the stomach and the drug PRX-3140 is released thereafter, for example after 2 hours post dose, 3 hours post dose, 4 hours post dose, 5 hours post dose, 6 hours post dose, 7 hours post dose, 8 hours post dose, etc., in the intestines at a slower rate. The surprisingly reduced interconversion of the PRX-3140 based on the formulations disclosed herein provide unexpected benefits in the administration of PRX-3140, or a polymorph or a salt thereof.

As a non-limiting example, the present disclosure provides sustained release compositions of the present disclosure in the form of solid dosage forms such as enteric coated delayed release tablets. A sustained release composition in the form of a tablet as described above serve as core (referred to as “core”, “core tablet” or “tablet core” hereinafter and used interchangeably) that is substantially covered by at least one layer of a functional coating. In other words, an enteric coated delayed release tablet of the present disclosure has a core which includes (i) PRX-3140 or a salt or polymorph thereof, (ii) at least one sustained-release agent, and (iii) at least one lubricant, and the core is substantially covered with at least one layer of a functional coating.

In some embodiments, the functional coating comprises at least one controlled release agent, such as a delayed release agent targeting the intestines (e.g., duodenum) or colon or both. The present disclosure provides that the functional coating may further comprise additional excipients such as plasticizers, anti-tacking agents, and the like. The addition of a delayed release agent to the functional coating provided surprisingly advantageous results, resulting in a substantial decrease in conversion of PRX-3140 to PRX-3140. The surprising advantage of coating the compositions (e.g., the tablets) with a coating comprising a delayed release agent evidences that such coatings can facilitate delivery of PRX-3140 more efficiently. Furthermore, it was surprisingly found that the coatings comprising a delayed releasing agent provided for a more consistent, slower release.

In certain embodiments, the functional coating of the present disclosure comprises at least one controlled release agent such as a delayed release agent, at least one plasticizer, and at least one anti-tacking agent/anti-adherent. In some embodiments, the enteric coated delayed release tablet may be coated with at least two coatings wherein at least one coating is a functional coating comprising a delayed release agent, at least one plasticizer, and at least one anti-tacking agent/anti-adherent.

Accordingly, in some embodiments the sustained release compositions comprising PRX-3140 or a polymorph or a salt thereof of the present disclosure comprise a core (e.g., a core tablet) that is substantially coated with at least one layer of a functional coating with a coating solution. The coating solution comprises at least one controlled release agent, such as a delayed release agent, at least one plasticizer, and at least one anti-tacking agent/anti-adherent. One of skill in the art will recognize that the core tablets disclosed herein can be enclosed in tablets, caplets or capsules and the like, and further coated with coating solutions disclosed herein to prepare other solid dosage forms such as tablet-in-tablets, tablet-in caplets, tablet-in-capsules and the like. In such cases, the sustained release compositions such as tablet-in-tablets, tablet-in caplets, tablet-in-capsules and the like will be substantially coated with at least one layer of functional coating as described herein. Coated tablets included inside other coated tablets, caplets and capsules can thus provide release of PRX-3140 or a polymorph or a salt thereof in pulses.

One of skill in the art will also recognize that, like the core tablets described herein, the other solid dosage forms of sustained release compositions such as mini-tablets, spheres, beads, granules, pellets, pills and the like, can also be used as cores for the preparation of enteric coated delayed release tablets, caplets, and capsules. Examples of such sustained release compositions include, but are not limited to, sustained release enteric tablets, enteric mini-tablets, enteric caplets, enteric beads, enteric spheres, enteric granules, enteric pellets, enteric pills, enteric tablet-in-tablet, enteric tablets-in-capsule, and the like and are used for delayed release of PRX-3140 or a polymorph or a salt thereof into the small intestines or colon or both. Commercially available delayed release capsules such as those available from Capsugel (e.g., VCaps® Plus or Enprotect® enteric capsules), can be used to prepare enteric coated delayed release capsules and are encompassed in the present disclosure. In some embodiments, the enteric delayed release capsules can be non-animal based capsules, such as a hypromellose capsule (for example, commercially available self-gelling Vcaps, VCaps Plus, VCaps enteric, other enteric capsules made using Xcellodose, ENCODE colonic delivery technology, and EnTrinsic™ drug delivery technology from Capsugel). Other technologies known in the art and available commercially (for example, Qualicaps, USA, Nutrascience, USA, etc.) for the formulating enteric forms of oral solid dosage forms can also be utilized.

The present disclosure provides that functional coating of a core can be performed with a coating solution which includes, but is not limited to, at least one controlled release agent, such as a delayed release agent. In some embodiments, the functional coating solution can comprise plasticizers, anti-tacking agents, and the like. In some embodiments, functional coating of a core can be performed with a coating solution which includes, but is not limited to, at least one controlled release agent, such as a delayed release agent at least one plasticizer, and at least one anti-tacking agent/anti-adherent. The coating solutions provided herein can be applied as films, such as thin films, on a range of solid dosage forms including particles, beads, spheres, granules, pellets, tablets, mini-tablets, pills, multiparticulates and capsules (with hard and soft shells) encapsulating the core (for example, immediate release capsules) to prepare the enteric coated delayed release solid dosage forms using the sustained release compositions described herein.

Where a sustained release composition, such as an enteric coated delayed release tablet, shows percentage dissolution ranging from about 0% to about 35% at 3 hours, from about 35% to about 55% at 12 hours, and from about 65% to about 85% at 24 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium, the at least one layer of functional coating provides from about 1% to about 60%, from about 2% to about 40%, from about 5% to about 30%, and from about 6% to about 20% weight gain of over the weight of the core (e.g., core tablet).

Where a sustained release composition, such as an enteric coated delayed release tablet, shows percentage dissolution of NMT about 5% at 2 hours, NLT about 60% at 3 hours, NLT about 95% at 6 hours and about 100% at 12 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium, the at least one layer of functional coating provides from about 1% to about 60%, from about 2% to about 40%, from about 5% to about 30%, and from about 6% to about 20% weight gain of over the weight of the core (e.g., core tablet).

Where a sustained release composition, such as an enteric coated delayed release tablet, shows percentage dissolution NMT about 10% at 3 hours, NLT about 30% at 6 hours, NLT about 50% at 9 hours, and NLT about 80% at 14 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium, the at least one layer of functional coating provides from about 1% to about 60%, from about 2% to about 40%, from about 5% to about 30%, and from about 6% to about 20% weight gain of over the weight of the core (e.g., core tablet).

Tablet weight will also vary in accordance with, among other things, the dosage of the compound PRX-3140, or a polymorph or a salt thereof, the type and amount of sustained release agent used, and the presence, types and amounts of additional materials. For dosages of the compound from about 0.5 mg to about 200 mg; tablet weights can range from about 10 mg to about 2000 mg per tablet. In some embodiments, the tablet weight ranges from 50 mg to 500 mg. In some embodiments, the enteric coated delayed release tablet has a tablet weight ranging from about 101 mg to 160 mg, from 90 mg to 110 mg, or from about 105 mg to about 120 mg. For time delay or delayed-release pharmaceutical preparations of sustained release composition of the present disclosure in the form of oral dosage forms, and acid-insoluble polymers (e.g., polymethacrylates) can be used as delayed release agent in the coating solution.

For enteric coated delayed-release preparations, cellulosic polymers such as glyceryl monostearate, glyceryl distearate, hydroxypropyl cellulose, hydoxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT® (Evoniks, Rohm Pharma; Westerstadt, Germany), including methacrylic acid-ethyl acrylate copolymer (1:1) (EUDRAGIT® L30D-55) and L100-55 (soluble at pH 5.5 and above). EUDRAGIT® 1,100D (soluble at pH 6.0 and above), EUDRAGIT® S (soluble at pH 7.0 and above, as a result of a higher degree of esterification), and EUDRAGIT® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; polyvinyl acetate/polyvinyl pyrrolidone, and enzymatically degradable polymers such as azo polymers, xanthan gum, chitosan, amylase and guar gum; zein and shellac.

In certain embodiments, the delayed release agent is a pH-sensitive polymer such as a polymethacrylate. In some embodiments, the delayed release agent is methacrylic acid-ethyl acrylate copolymer (1:1) USP-NF (EUDRAGIT® L30D-55). In some embodiments, the delayed release agent is methacrylic acid-ethyl acrylate copolymer (1:2).

The present disclosure also provides that combinations or mixtures of different delayed release agents may be used in a single layer of functional coating. In some embodiments, multilayer (e.g., two or more layers) coatings using different delayed release agents or polymers may also be applied to the core.

When a sustained release composition in the form of an enteric coated delayed release dosage form (such as an enteric coated delayed release tablet) releases PRX-3140 or polymorphs salts thereof in a sustained manner for at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16 hours, at least 18 hours, at least 24 hours, at least 48 hours, or at least 72 hours is obtained, the delayed release agent, such as methacrylic acid-ethyl acrylate copolymer (1:1) USP-NF (EUDRAGIT® L30D-55 is present in the at least one layer of a functional coating in an amount ranging from about 0.1% to about 35%, from about 5% to about 35%, or from about 8% to about 20% w/w of the weight of the core (e.g., core tablet).

When a sustained release composition of the present disclosure, such as a delayed release tablet, shows percentage dissolution ranging from about 0% to 35% at 3 hours, from about 35% to about 55% at 12 hours, and from about 65% to 85% at 24 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium, the delayed release agent is present in the at least one layer of coating in amounts ranging from about 0.1% to about 30%, from about 5% to about 25%, or from about 8% to about 14% w/w of the weight of the core tablet. In some embodiments, the delayed release agent is present in the at least one layer of coating in amounts ranging from about 0.1% to about 20% on a w/w basis relative to the weight of the core tablet.

When a sustained release composition of the present disclosure, such as a delayed release tablet, shows percentage dissolution of NMT about 5% at 2 hours, NLT about 70% at 3 hours, NLT about 95% and about 100% at 12 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium, the delayed release agent is present in the at least one layer of coating in amounts ranging from about 0.1% to about 30%, from about 5% to about 25%, or from about 8% to about 14% w/w of the weight of the core tablet. In some embodiments, the delayed release agent is present in the at least one layer of coating in amounts ranging from about 0.1% to about 20% on a w/w basis relative to the weight of the core tablet.

Such a release of PRX-3140 or polymorphs salts thereof from the sustained release composition of the present disclosure is generally initiated in the intestines about 2 hours post-dose.

At least one plasticizer is added to control the softness or pliability of the sustained release compositions prepared as solid dosage forms, such as an enteric coated delayed release tablet or a shell of a capsule or a caplet, and thus, may improve the mechanical properties of the pH-sensitive materials of the coatings on the solid dosage forms.

Suitable plasticizers, include petroleum oils (for e.g., a paraffinic process oil, a naphthenic process oil, and an aromatic process oil), squalene, squalane, plant oils, (e.g., olive oil, camelia oil, castor oil, tall oil, and a peanut oil), mineral oils, silicon oils, dibasic or phthalic acid esters, (e.g., dibutyl phthalate, diethyl phthalate, and dimethyl phthalate, and dioctyl phthalate), liquid rubbers (e.g., polybutene and a liquid isoprene rubber), liquid fatty acid esters (e.g., isopropyl myristate ISM), hexyl laurate, diethyl sebacate, and diisopropyl sebacate, citric acid esters such as triethyl citrate, tributyl citrate, acetyl tributyl citrate, and acetyl triethyl citrate, triacetin, propylene glycol, diethylene glycol, polyethylene glycols, polypropylene glycol, other polyethylene oxide sorbitan esters, phthalates, sorbitol, glycol salicylate, crotaminton, glycerin, PLASACRYL™ T20, or mixtures thereof.

In some embodiments, at least one plasticizer in the functional coating is triethyl citrate. In some embodiments, the at least one plasticizer (e.g., citric acid esters such as triethyl citrate) is present in amounts ranging from about 0.01% to about 5%, from about 0.1% to about 4%, from about 0.2% to about 2%, or from about 0.5% to about 1.5% w/w of the weight a core tablet.

The present disclosure provides that at least one layer of functional coating includes at least one anti-tacking/anti-adherent agent. Anti-tacking agent/anti-adherent is included in the coating solution for coating to prevent sticking of the tablets to punch faces and prevent sticking to machine dosators, tamping pins, etc. Exemplary anti-tacking agent include talc, glyceryl monostearate, colloidal silicon dioxide, kaolin, or any combination thereof.

The addition of talc as an anti-tacking agent to the functional coating provided surprisingly advantageous results, resulting in a substantial decrease in conversion of PRX-3140 to PRX-3140. The surprising advantage of coating the compositions (e.g., the tablets) with a coating comprising talc evidences that such coatings can facilitate delivery of PRX-3140 more efficiently. Furthermore, it was surprisingly found that the coatings comprising a delayed releasing agent provided for a more consistent, slower release.

The sustained release compositions (e.g., enteric coated delayed release tablets) include at least one anti-tacking agent present in amounts ranging from about 0.1% to about 10% of w/w, from 1% to about 8%, or from about 2% to about 6% w/w of core. Exemplary enteric coated delayed release capsules and tablets are provided herein and in Examples 16 and 17.

An exemplary sustained release composition in the form of an enteric coated delayed release tablet comprises a core tablet, wherein the core tablet comprises: about 0.01% to about 40% or about 0.5 mg to about 200 mg of PRX-3140 or a polymorph or a salt thereof, hypromellose, microcrystalline cellulose, and magnesium stearate; the core tablet being substantially enclosed in at least one layer of a functional coating, wherein the at least one layer of functional coating comprises methacrylic acid-ethyl acrylate copolymer (ranging from 2:1 to 1:2), triethylcitrate, and talc.

An exemplary sustained release composition in the form of an enteric coated delayed release tablet, comprises a core tablet wherein the core tablet comprises: (i) about 0.01% to about 40% PRX-3140 or a polymorph or a salt thereof, (ii) about 0.1% to about 99% hypromellose; (iii) about 1% to about 99% microcrystalline cellulose; and (iv) about 0.01% to about 5% magnesium stearate; the core tablet being substantially enclosed in at least one layer of functional coating; wherein the at least one layer of functional coating comprises: (i) about 5% to about 40% methacrylic acid-ethyl acrylate copolymer (1:1) (polymethacrylate Methacrylic Acid Copolymer Dispersion, 30% solids); wherein the at least one layer of functional coating contributes to about 2% to about 20% weight gain over the average core tablet weight. The amounts are expressed in terms of percentage by weight based on the weight of the core tablet. In some embodiments, the foregoing sustained release composition has a drug (PRX-3140 or a polymorph or a salt thereof) to sustained release agent ratio of 1:5.

An exemplary sustained release composition in the form of an enteric coated delayed release tablet, comprises a core tablet wherein the core tablet comprises: (i) about 0.01% to about 40% PRX-3140 or a polymorph or a salt thereof, (ii) about 5% to about 60% hypromellose; (iii) about 10% to about 90% microcrystalline cellulose; and (iv) about 0.01% to about 5% magnesium stearate; the core tablet being substantially enclosed in at least one layer of functional coating; wherein the at least one layer of functional coating comprises: (i) about 5% to about 40% methacrylic acid-ethyl acrylate copolymer (1:1) (polymethacrylate Methacrylic Acid Copolymer Dispersion, 30% solids); wherein the at least one layer of functional coating contributes to about 2% to about 20% weight gain over the average core tablet weight. The amounts are expressed in terms of percentage by weight based on the weight of the core tablet.

An exemplary sustained release composition in the form of an enteric coated delayed release tablet, comprises a core tablet wherein the core tablet comprises: (i) about 0.01% to about 40% PRX-3140 or a polymorph or a salt thereof, (ii) about 5% to about 60% hypromellose; (iii) about 10% to about 90% microcrystalline cellulose; and (iv) about 0.01% to about 5% magnesium stearate; the core tablet being substantially enclosed in at least one layer of functional coating; wherein the at least one layer of functional coating comprises: (i) about 0.1% to about 20% methacrylic acid-ethyl acrylate copolymer (1:1) (polymethacrylate Methacrylic Acid Copolymer Dispersion, 30% solids), (ii) about 0.01% to about 5% triethyl citrate, and (iii) about 1% to about 10% talc; wherein the at least one layer of functional coating contributes to about 2% to about 20% weight gain over the average core tablet weight. The amounts are expressed in terms of percentage by weight based on the weight of the core tablet.

An exemplary sustained release composition in the form of an enteric coated delayed release tablet comprises a core tablet, wherein the core tablet comprises: (i) 0.5 mg, 1 mg, 2 mg, 4 mg, 5 mg, 6 mg, 8 mg, 10 mg, 20 mg or 40 mgs of PRX-3140 or a polymorph or a salt thereof, (ii) about 0.1% to about 99% or about 5% to about 60% hypromellose; (iii) about 1% to about 99% or about 10% to about 90% microcrystalline cellulose; and (iv) about 0.01% to about 5% magnesium stearate; the core tablet being substantially enclosed in at least one layer of a functional coating; wherein the at least one layer of the functional coating comprises: (a) about 0.1% to about 20% methacrylic acid-ethyl acrylate copolymer (1:1) (polymethacrylate Methacrylic Acid Copolymer Dispersion, 30% solids), (b) about 0.01% to about 5% triethyl citrate, and (c) about 1% to about 10% talc; wherein the at least one layer of functional coating contributes to about 2% to about 20% weight gain over the average core tablet weight. The amounts of all components from (ii) to (iv) and from (a) to (c) are expressed in terms of percentage by weight based on the weight of the core tablet. Such an enteric coated delayed release tablet (i) provides a sustained release of PRX-3140 or a polymorph or a salt thereof over a time period of at least 2 hours, at least 3 hours, at least 4 hours, at least 6 hours, at least 7 hours, at least 8 hours, at least 9 hours, at least 10 hours, at least 12 hours, at least 14 hours, at least 16 hours, at least 18 hours, at least 24 hours, at least 48 hours, or at least 72 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium.

In some embodiments, such an enteric coated delayed release tablet shows a percentage dissolution of about 0% to about 30% at 3 hours, about 30% to about 50% at 12 hours, and about 65% to about 85% after 12 hours in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium.

In some embodiments, such an enteric coated delayed release tablet shows in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium a percentage dissolution: (i) ranging from about 0% to 35% at 3 hours, from about 35% to about 55% at 12 hours, and from about 65% to 85% at 24 hours, (ii) of NMT 30% at 3 hours, NMT 50% at 12 hours, and NLT 80% at 24 hours; or (iii) of at least 20% to 30% after 24 hours.

In some embodiments, such an enteric coated delayed release tablet shows in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium a percentage dissolution of NMT about 5% at 2 hours, NLT about 70% at 3 hours, NLT about 95% at 6 hours and about 100% at 12 hours.

In some embodiments, such an enteric coated delayed release tablet shows in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium a percentage dissolution of NMT about 5% at 2 hours, NMT about 10% at 3 hours, NLT about 30% at 6 hours, NLT about 50% at 9 hours, and NLT about 80% at 14 hours.

In some embodiments, such an enteric coated delayed release tablet shows in a dissolution test according to the 50 RPM USP paddle method and using water or pH 1.2 at 37 degrees Celsius for 2 hours in simulated gastric fluid and pH 6.8 at 37 degrees Celsius for 24 hours in simulated intestinal fluid as a test medium a percentage dissolution of NMT about 10% at 3 hours, NLT about 30% at 6 hours, NLT about 50% at 9 hours, and NLT about 80% at 14 hours.

Functional coatings of sustained release compositions of the present disclosure, including the enteric coated delayed release tablets, may further include a pharmaceutically acceptable excipient. Pharmaceutically acceptable excipient may be any additive conventionally used in the technical field of preparation such as fillers, stabilizers, glidants, surfactants, light shielding agents, sweeteners, colorants, flavorants, anti-oxidants, preservatives, reducing agent, chelating agent and the like described herein. The pharmaceutically acceptable excipients are used in an amount conventionally used in the technical field of preparation. In addition, two or more kinds of these pharmaceutically acceptable excipients may be mixed at an appropriate ratio and used. In an aspect, the present disclosure provides that the sustained release compositions upon release of the compound PRX-3140 or a polymorph or a salt thereof provide a sustained exposure of the subject to the compound upon oral administration from about 2 hours to about 270 hours per dose. In some embodiments, the sustained release compositions provide a sustained exposure of the subject to the released compound PRX-3140 or a polymorph or a salt thereof for at least 2 hours, at least 4 hours, at least 6 hours, at least 8 hours, at least 10 hours, at least 12 hours, at least 14 hours, at least 18 hours, at least 20 hours, at least 24 hours, at least 36 hours, at least 48 hours, at least 72 hours, at least 96 hours, at least 120 hours, at least 160 hours, at least 180 hours, at least 200 hours, at least 220 hours, at least 250 hours and at least 270 hours.

### VII. Pharmacokinetic Characteristics

In an aspect, sustained release compositions of the present disclosure comprising compound PRX-3140 or a polymorph or a salt thereof can provide enhanced pharmacokinetics as compared to the immediate release product, or “drug-in-capsule”, in a subject upon oral administration. In addition, a sustained release composition may also produce less side effects.

Provided herein are pharmacokinetic (PK) parameters of a single dose of sustained release tablets in the form of enteric coated delayed release tablets of the present disclosure. As non-limiting example, for certain embodiments, a single oral dose of a sustained release composition (for example in the form of an sustained release tablet or capsule) ingested by a subject can provide or is capable of achieving a mean plasma Cmax (ng/mL) of PRX-3140 acid in the blood, serum or plasma of at least about 30%, at least about 35%, at least about 40%, at least about 45%, at least about 50%, at least about 55%, at least about 60%, at least about 65%, at least about 70% at least about 75% at least about 80% at least about 85%, and at least about 90% of the Cmax of an equivalent single oral dose of the immediate release capsule. In certain embodiments, a single dose of orally ingested sustained release tablet or capsule of the present disclosure can provide or is capable of achieving a mean plasma Cmax (ng/mL) of at least about 45% of the Cmax of an orally ingested equivalent single dose of the immediate-release capsule.

The present disclosure provides that a single oral dose of sustained release compositions such as the enteric coated delayed release tablets of the present disclosure ingested by a subject can provide or is capable of achieving a mean plasma Tmax (hr) greater than the mean plasma Tmax (hr) of an equivalent single oral dose of the IR dosage form. As a non-limiting example, the mean plasma Tmax (hr) of a single oral dose of an enterically coated delayed release tablet ingested by a subject in fasting state is greater than the mean plasma Tmax (hr) of an equivalent single oral dose of IR dosage form. Similarly, in certain embodiments, a single oral dose of the sustained release compositions of the present disclosure, for example in the form of enteric coated delayed release tablets, can have a mean plasma Tmax (hr) that is at least 0.1, at least 0.5, at least 1, at least 1.5, at least 2, at least 2.5, at least about 3, at least about 3.5, at least about 4, at least about 4.5, at least 5, at least about 5.5, at least about 6, at least about 6.5, at least about 7, at least about 7.5, at least about 8, at least 8.5, at least 9 time, at least 9.5 and at least 10 times greater than that of an equivalent single oral dose of the reference product, the IR dosage form. Thus, the sustained release compositions of the present disclosure reach their mean plasma Cmax considerably later than the reference product, the IR dosage form (See Table 15). Mean plasma concentrations over time following a single dose of a sustained release composition and the reference product, the IR dosage form, is provided in Table 12. In certain embodiments, the sustained release compositions of the present disclosure in the form of enteric coated delayed release tablet of hardness can have a mean plasma Tmax (hr) that is at least about 6 to about 10 time greater than the reference product, the IR dosage form.

In some embodiments, a sustained release composition of the present disclosure (e.g., an sustained-release tablet or capsule) may produce a mean Tmax of a compound (e.g., PRX-3140 acid) of at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 26 hours, at least about 28 hours, at least about 30 hours, at least about 32 hours, at least about 34 hours, at least about 36 hours, at least about 38 hours, at least about 40 hours, at least about 42 hours, at least about 44 hours, at least about 46 hours, at least about 48 hours, at least about 50 hours, at least about 55 hours, at least about 60 hours, at least about 65 hours, at least about 70 hours, at least about 75 hours, or at least about 80 hours in a plasma or a serum of a subject orally administered the sustained composition. In some embodiments, a sustained release composition may produce a mean Tmax of the compound of from 4 hours to 80 hours, from 5 hours to 80 hours, from 6 hours to 80 hours, from 7 hours to 80 hours, from 8 hours to 80 hours, from 9 hours to 80 hours, from 10 hours to 80 hours, from 15 hours to 80 hours, from 24 hours to 80 hours, from 36 hours to 80 hours, from 48 hours to 80 hours, from 4 hours to 70 hours, from 5 hours to 70 hours, from 6 hours to 70 hours, from 7 hours to 70 hours, from 8 hours to 70 hours, from 9 hours to 70 hours, from 10 hours to 70 hours, from 15 hours to 70 hours, from 24 hours to 70 hours, from 36 hours to 70 hours, from 48 hours to 70 hours, from 4 hours to 60 hours, from 5 hours to 60 hours, from 6 hours to 60 hours, from 7 hours to 60 hours, from 8 hours to 60 hours, from 9 hours to 60 hours, from 10 hours to 60 hours, from 15 hours to 60 hours, from 24 hours to 60 hours, from 36 hours to 60 hours, from 48 hours to 60 hours, from 4 hours to 55 hours, from 5 hours to 55 hours, from 6 hours to 55 hours, from 7 hours to 55 hours, from 8 hours to 55 hours, from 9 hours to 55 hours, from 10 hours to 55 hours, from 15 hours to 55 hours, from 24 hours to 55 hours, from 36 hours to 55 hours, or from 48 hours to 55 hours in a plasma or a serum of a subject orally administered the sustained composition.

Further, the higher Tmax and the drug release profile of the sustained release compositions as compared to the IR dosage form suggest that the rate of release and the rate of absorption or rate of uptake (as used interchangeably in the present disclosure) of the compound (PRX-3140 or a polymorph or a salt thereof) into the blood of a subject from an orally administered sustained release composition is also slower as compared with the rate of release and the rate of absorption of the compound from the IR dosage form. In certain embodiments, the present disclosure provides that the sustained release compositions, for example, in the form of sustained release tablets or capsules such as enterically coated delayed release capsules, are capable of achieving one or more of the pharmacokinetic parameters and mean plasma concentration as disclosed in Table 13 following oral administration of a single and multiple doses of the sustained release composition to a subject. In some embodiments, the mean Tmax of sustained release compositions of the present disclosure after a single dose is about 5 to 8 hours.

Provided herein are exemplary pharmacokinetic parameters of multiple doses (for example, a once daily dose) of sustained release tablets or capsules in the form of enteric coated delayed release tablets or capsules of the present disclosure.

Similarly, in certain embodiments, the sustained release compositions of the present disclosure (for example, in the form of sustained release tablets such as enteric coated delayed release tablets) can provide or is capable of achieving a mean plasma Cmax (ng/mL) of at least about 30%, at least about 35%, at least about 40%, at least about 45%, at least about 50%, at least about 55%, at least about 60%, at least about 65%, at least about 70% at least about 75% at least about 80% at least about 85%, and at least about 90% of the Cmax of the IR dosage form when the sustained release compositions and IR dosage form are orally dosed with multiple equivalent doses (as non-limiting example, for 14 days 1 mg once daily) to subjects. In certain embodiments, the sustained release compositions of the present disclosure, for example, in the form of enteric coated delayed release tablets of the present disclosure, can provide or are capable of achieving a mean plasma Cmax (ng/mL) of at least about 45% of the Cmax of the IR dosage form when the sustained release compositions and IR dosage form are orally dosed with multiple equivalent doses (as non-limiting example, for 14 days 4 mg once daily) to subjects in a fasting state.

Mean time to reach steady state levels of PRX-3140 acid in the blood, serum or plasma of the subject thereof upon multiple doses of a sustained release composition of the present disclosure is about 14 days. At steady state, the sustained release compositions of the present disclosure show less fluctuation over the inter-dosing interval compared with reference product, the IR dosage form.

A composition of the present disclosure (e.g., a composition comprising PRX-3140) may produce an average concentration at steady state (Cavg) of PRX-3140 acid within a subject. In some embodiments, the composition may produce an average concentration steady state (Cavg) of from 1 ng/mL to 10 ng/mL, from 5 ng/mL to 15 ng/mL, from 10 ng/mL to 20 ng/mL, from 15 ng/mL to 25 ng/mL, from 20 ng/mL to 30 ng/mL, from 25 ng/mL to 35 ng/mL, from 30 ng/mL to 40 ng/mL, from 35 ng/mL to 45 ng/mL, from 40 ng/mL to 50 ng/mL, from 45 ng/mL to 60 ng/mL, from 50 ng/mL to 70 ng/mL, from 60 ng/mL to 80 ng/mL, from 70 ng/mL to 90 ng/mL, from 80 ng/mL to 100 ng/mL, from 90 ng/mL to 110 ng/mL, from 100 ng/mL to 120 ng/mL, from 110 ng/mL to 130 ng/mL, from 120 ng/mL to 140 ng/mL, or from 130 ng/mL to 150 ng/mL in a subject. In some embodiments, the composition may produce an average concentration steady state (Cavg) in a subject of from 1 ng/mL to 10 ng/mL, from 5 ng/mL to 15 ng/mL, from 10 ng/mL to 20 ng/mL, from 15 ng/mL to 25 ng/mL, from 20 ng/mL to 30 ng/mL, from 25 ng/mL to 35 ng/mL, or from 30 ng/mL to 40 ng/mL per milligram of PRX-3140 administered to the subject.

In certain embodiments, the present disclosure provides that the sustained release compositions, for example, in the form of a sustained release capsules, are capable of achieving one or more of the pharmacokinetic parameters shown in Table 13 following oral administration of multiple doses to a subject or at steady state. In other embodiments, the median Tmax of sustained release compositions of the present disclosure after multiple doses or at steady state range from 3 to 24 hours. In at least one embodiment, the median Tmax of the sustained release compositions of the present disclosure after multiple doses or at steady state is 8.7 hours.

The present disclosure provides that in some embodiments, the relative bioavailability of the compound from the sustained release compositions of the present disclosure such as the enteric coated delayed release tablets when dosed orally in a single and/or multi dose ranges from about at least 50% to about at least 150% as compared to equivalent, single or multiple dose(s) of the IR dosage form orally administered to subjects. The sustained release compositions in the form of sustained release tablets or capsules having bioavailability of the compound with improved pharmacokinetic parameters (such as longer plasma and serum Tmax) and low % fluctuation of the compound (PRX-3140 acid) as disclosed herein (See Example 15) may be desirable for longer term use for treatment of Alzheimer's disease, and for continued use and compliance by the subjects.

Thus, the present disclosure provides that the compound (PRX-3140 or a polymorph or a salt thereof) is absorbed and systemically available in a controlled sustained manner after 2 hours. The present disclosure also provides that the sustained release compositions display dose proportionality in peak drug concentrations in plasma and serum and AUC cures such as AUC24 over the dose range of 0.1 mg to about 500 mg.

In another aspect, provided herein are methods of treating a subject having or at risk of having Alzheimer's disease, the method comprising the steps of (a) orally administering to a subject a sustained release composition of the present disclosure, (b) obtaining or having obtained a biological sample such as whole blood, plasma, and/or serum from the subject, and (c) measuring or having measured PRX-3140 acid in the subject's whole blood, plasma, and/or serum over a period of time, for example, at least 6 hours, at least 12 hours, at least 24 hours, at least 48 hours, at least 72 hours, at least 120 hours, at least 7 days, or at least 14 days, and (d) orally administering a different sustained release composition of the present disclosure to the subject, wherein the sustained release composition is capable of achieving one or more parameters of any one or more of Table 12 and Table 13.

Accordingly, provided herein are methods of maintaining in a subject in need thereof a plasma PRX-3140 acid level greater than 1 nM by administering orally to the subject a sustained release composition comprising PRX-3140 or a polymorph or a salt thereof. In some embodiments, the subject's plasma PRX-3140 acid level is maintained at a steady state level greater than 10 nM. In some embodiments, the subject's plasma PRX-3140 acid levels are maintained at a steady state level ranging from 1 nM to 1000 nM (for example, from 1 nM to 500 nM, from 1 nM to 300 nM, from 1 nM to 200 nM, from 1 nM to 100 nM, or from 1 nm to 50 nM). In some embodiments, the subject's plasma PRX-3140 acid levels are maintained at a steady state level >1 nM.

In one embodiment, the invention relates to an sustained-release dosage form which when administered once daily in steady state to a healthy subject or a patient, the % fluctuation of the blood concentrations for total and/or free PRX-3140 acid measured as (Cmax−Cmin)/Caverage is less than the fluctuation observed when administering the IR dosage form in a once daily regimen and being determined under similar conditions and administered in similar molecular daily dosages of the crystalline PRX-3140. The decrease is preferable at least 10%, such as at least 20%, preferable at least 30%, such as at least 40%, more preferred at least 50%.

In one embodiment, the invention relates to a sustained-release dosage form which when administered to at least 6 healthy subjects in fasted state, the mean residence time PRX-3140 acid measured in blood is at least 50% longer than the mean residence time measured under similar conditions with the IR dosage form.

In a yet further aspect the present invention provides for a method for providing immunosuppressive treatment of a patient in need thereof in a once daily regimen by administration of a sustained release formulation as described herein an providing one or more of decreased Cmax, decreased % fluctuation, increased AUC, longer time to Tmax, and a higher Cmin once steady-state is achieved. Additionally, the methods provides for a Cmin which correlates to the bioavailability with a correlation factor of at least 0.75 to 1, such as at least 0.80, preferably 0.85, more preferred 0.90, still more preferred at least 0.95, and even more preferred of at least 0.97.

Jn a preferred embodiment the difference in bioavailability is substantially independent of the time of the day the dosage is administered. This provides the possibility of a once daily dosage regimen at bedtime or in the evening in addition to the normal morning dosing. However more importantly, the risk of decreased exposure if the patient actually ingest the dosage form on a different time than prescribed and expected (non-compliance by the patient), the risk for the patient is decreased for reduced exposure and thereby increased risk of side effects.

The present invention is further illustrated by reference to the Examples below. However, it should be noted that these Examples, like the embodiments described above, are illustrative and are not to be construed as restricting the enabled scope of the invention in any way.

### VII. Methods of Making Pharmaceutical Compositions

In further embodiments, the present disclosure provides a method of making a pharmaceutical composition wherein the method further comprises formulating the particles.

In certain embodiments, the particles are formulated into unit doses such as tablets or capsules.

In some embodiments wherein the pharmaceutical compositions further comprises at least one excipient, the present disclosure also provides a method of making a pharmaceutical composition wherein the method further comprises mixing the particles with at least one excipient to form a second mixture; and formulating the second mixture.

In certain embodiments, the particles are formulated into unit doses such as tablets or capsules.

### III. Methods of Treatment

The present disclosure is directed to compositions and methods for reducing body weight, maintaining body weight, reducing body weight gain, altering body composition, treating diabetes, lower fasting glucose, lowering HbA1c, reducing average daily blood glucose, or lowering postprandial glucose in a subject in need or desirous thereof, by chronically administering crystalline PRX-3140 potassium salt by oral sustained-release compositions. The methods contemplate the chronic or sustained administration of an effective amount of crystalline PRX-3140 potassium salt by oral administration to a subject to affect the desired results as described herein. As used herein in the context of treating diabetes, reducing HbA1c, controlling postprandial blood glucose, lowering fasting glucose and reducing overall daily blood glucose concentration, a subject in need thereof may include subjects with diabetes, impaired glucose tolerance, insulin resistance, or subjects unable to auto-regulate blood glucose. In further embodiments, the subject is in need of or desirous of a reduction in body weight.

It is also contemplated that the methods disclosed herein are useful in maintaining levels of PRX-3140 that control fasting blood glucose with limited effects on, no detectable effects on or without inducing weight loss, reducing appetite, slowing gastric emptying, or exerting postprandial glucose level control. In particular, the methods disclosed herein are useful in controlling fasting blood glucose levels without inducing weight loss. Patient populations benefiting from controlled fasting blood glucose levels without accompanying weight loss include, but are not limited to, elderly patients, patients with human immunodeficiency virus (HIV) infections, or other patients where weight loss is contraindicated. Additionally is provided a method for reducing the increase in postprandial blood glucose concentration compared to preprandial blood glucose concentration, such that the difference between blood glucose concentration before and after a meal is reduced. This results in a lessening of the variation in blood glucose concentrations during the day as determined, for example, by 7 point self monitored blood glucose as described herein. Any method for determining circulating blood concentrations of glucose in a patient may be employed with the claimed methods.

Likewise in the area of glycemic control, crystalline PRX-3140 potassium salt sustained-release formulations can be administered orally according to the methods disclosed herein for a period sufficient to achieve a target HbA1c, a target fasting glucose level, a target overall daily blood glucose concentration, etc. after which the plasma concentration of PRX-3140 acid may be reduced to a maintenance level or discontinued. If discontinued, the administration can be resumed later if necessary. In one embodiment, crystalline PRX-3140 potassium salt sustained-release formulations are administered according to methods disclosed herein for a period sufficient to lower or stabilize fasting glucose levels, reducing or eliminating high or higher than desired fasting glucose levels.

In some embodiments, methods disclosed herein further provide that crystalline PRX-3140 potassium salt is co-administered with one or more oral diabetic agents. Such agents include, but are not limited to metformin, a sulphonylurea (SU), a thiazolidinedinoe (TZD) or any combination thereof. Exemplary agents include pioglitazone, rosiglitazone, glibenclamide, gliclazide, glimepiride, glipizide, gliquidone, chlorpropamide, and tolbutamide. Crystalline PRX-3140 potassium salt sustained-release formulations can also be co-administered with insulin. Co-administration can be achieved by any suitable means or dosing regimen.

### EXAMPLES

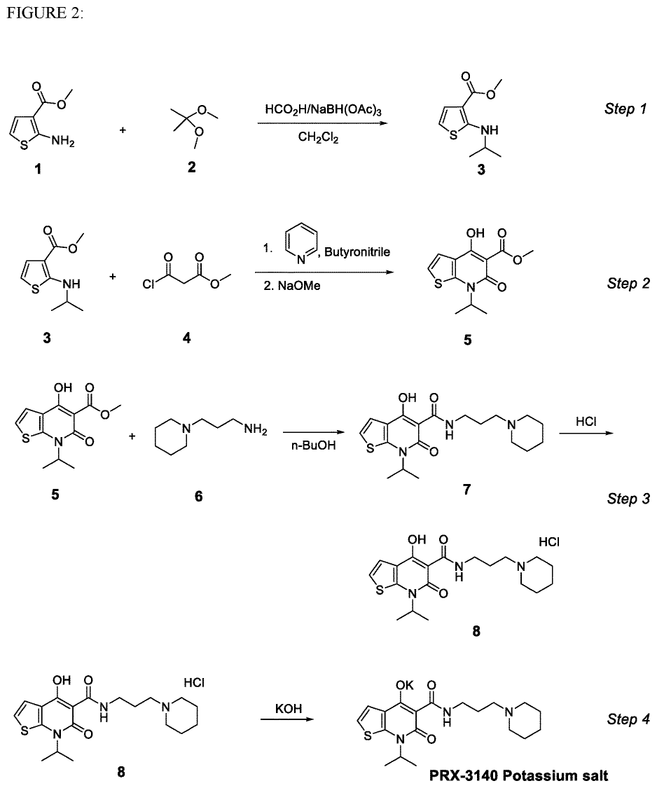

Example 1—Small-scale preparation of 50 grams of crystalline PRX-3140 potassium salt. U.S. Pat. Nos. 7,488,736 and 7,982,040 described PRX-3140 preparation in a six-step process. Supply of 3-piperidin-1-yl-propylamine allows synthesis of crystalline fine particle Form I of PRX-3140 potassium salt in four-step process at the 50-gram scale as shown in FIG. 2.

Step 1. Reductive amination—Methyl 2-iso-propylaminothiophene-3-carboxylate (3). To a 3 L three-neck round-bottom flask equipped with a mechanical stirrer was added methyl 2-aminothiophene-3-carboxylate (90.52 g, 0.5758 mol) and sodium triacetoxyborohydride (207.48 grams, 0.979 mole). Anhydrous dichloromethane (640 mL) was added and the mixture was stirred for 2 minutes prior to the addition of a solution of formic acid (53.01 grams, 1.1517 mole) and 2,2-dimeoxypropane (299.88 grams, 2.8793 mole) over 30 minutes at 15-32 degrees Celsius (internal temperature) under argon (cooled with cold water if needed). The addition funnel was rinsed with anhydrous dichloromethane (92 mL) and charged to the reaction mixture. After the addition was completed, the resulting mixture was stirred at 25-30 degrees Celsius for 2 hours. The reaction mixture was added to an aqueous solution of potassium hydroxide (304.1 gram, 4.607 mole) in water (905 mL) at 5 degrees Celsius and maintaining the temperature below 40 degrees Celsius Dichloromethane (100 mL) was used to rinse the reaction flask and added to the mixture. The resulting mixture was stirred at room temperature for 0.5 hour, then filtered, and the residue was washed with dichloromethane (300 mL). The filtrate was diluted with water (0.5 L), and the phases were separated. The aqueous phase was extracted with dichloromethane (0.5 L). The combined organic phase was mixed with water (0.5 L) and stirred for 0.5 hour. The phases were separated again, and the organic phase was dried over Na₂SO₄. Filtration and concentration provided 114.7 grams crude as red oil, which was dissolved in heptane (0.50 L) and stirred with neutral alumina (90 grams) for 0.5 hour, then filtered, and the filtrate was evaporated. The titled compound (105.8 g, 92%) was obtained as yellowish oil. HPLC purity: 98.2%. 1H NMR (300 MHz, CDCl₃) δ 7.35 (br s, 1H), 7.00 (d, J=5.7 Hz, 1H), 6.14 (dd, J=5.7, 1.2 Hz, 1H), 3.78 (s, 3H), 3.56-3.44 (m, 1H), 1.32 (s, 3H), 1.29 (s, 3H).

Step 2. Acylation and cyclization—Methyl 4-hydroxy-7-isopropyl-6-oxo-6,7-dihydro-thieno[2,3-b]pyridine-5-carboxylate (5). To a solution of starting amine 3 (89.7 grams, 0.450 mole) and pyridine (71.2 grams, 0.900 mole) in butyronitrile (0.9 L) was heated to 70-75 degrees Celsius with stirring under argon. A solution of methyl malonyl chloride (116.7 grams, 0.855 mol) in butyronitrile (0.45 L) was added drop-wise to the reaction mixture to maintain the temperature at 70-75 degrees Celsius (internal temperature) with vigorous stirring (with occasional turn off of the heating, especially in the beginning). At the end of addition, the temperature was held for 8 to 10 minutes before sodium methoxide solution (25%, 390.0 grams, 415 mL, 1.80 mol) was added over 10 minutes at the same temperature. The resulting mixture was stirred at the same temperature for 10 minutes, then cooled to room temperature. Water (0.45 L) was added, and the mixture was stirred for 20 minutes at room temperature. The phases were separated, and the organic phase was extracted with water (0.45 L). The combined aqueous phase was washed with EtOAc (2×0.45 L). The aqueous phase was then acidified to pH 4.0-4.5 with 1:5 v/v HCl/water at room temperature. The resulting solid was filtered and washed with water (2×180 mL). The filter cake was dried on vacuum oven at 45-50 degrees Celsius overnight. Title compound was obtained (68.48 g, 57%) as yellow solid. HPLC purity: 98.1%. The filtrate was cloudy overnight and 2nd filtration provided 2nd crop (9.68 g, 8%, HPLC purity: 87.4%), which was not used for next step. 1H NMR (300 MHz, CDCl3) δ 7.32 (d, J=5.7 Hz, 1H), 6.90 (d, J=6.0 Hz, 1H), 4.00 (s, 3H), 1.70-1.60 (m, 1H), 1.64 (s, 3H), 1.61 (s, 3H).

Step 3. Amidation and salt formation 4-Hydroxy-7-isopropyl-6-oxo-6,7-dihydro-thieno[2,3-b]pyridine-5-carboxylic acid (3-piperidin-1-yl-propyl)-amide hydrochloride (8). A mixture of methyl 4-hydroxy-7-isopropyl-6-oxo-6,7-dihydro-thieno[2,3-b]pyridine-5-carboxylate (5, 2 grams, 7.48 mmol) and 3-piperidin-1-yl-propylamine (6, 1.12 g, 7.85 mmol, 1.05 equiv.) was heated at 90-95 degrees Celsius for 2 hours. TLC analysis indicated that the reaction was complete. The reaction mixture was cooled to room temperature, diluted with water (5 mL), and charged with 1M HCl (7.4 mL). The organic layer was separated and washed with water (4 mL). The organic layer was treated with concentrated hydrochloric acid (1.4 grams). The organic phase was concentrated to ca. 11 mL (9.9 grams). The residue was charged with n-butanol (10 mL). The mixture was re-concentrated to a volume of ca. 11 mL (9.9 grams). The residue was heated to 50 degrees Celsius and diluted with MTBE (20 mL). The mixture was stirred at 50 degrees Celsius for 40 minutes, cooled to room temperature, and then held at −10 degrees Celsius overnight. The slurry was filtered at 0-5 degrees Celsius. The filter cake was washed with n-butanol/MTBE (1:3, 2×4 mL) and dried on rotavapor to give 4-hydroxy-7-isopropyl-6-oxo-6,7-dihydro-thieno[2,3-b]pyridine-5-carboxylic acid (3-piperidin-1-yl-propyl)-amide hydrochloride (8, 2.35 grams, yield: 75%, HPLC: 99.65%) as a white solid. The batch started with 66.35 g of ester 5 and 84.5 g of title compound 8 was obtained (yield: 82%). ¹H NMR (300 MHz, DMSO-d₆) δ 10.24 (br t, J=5.4 Hz, 1H), 9.91 (br s, 1H), 7.38 (d, J=5.7 Hz, 1H), 7.32 (d, J=5.7 Hz, 1H), 3.47-3.30 (m, 4H), 3.08-2.98 (m, 2H), 2.90-2.76 (m, 2H), 2.05-1.93 (m, 2H), 1.82-1.62 (m, 6H), 1.57 (s, 3H), 1.55 (s, 3H), 1.44-1.26 (m, 1H).

Step 4: Preparation of 4-Hydroxy-7-isopropyl-6-oxo-6,7-dihydro-thieno[2,3-b]pyridine-5-carboxylic acid (3-piperidin-1-yl-propyl)-amide potassium salt (PRX-3140 potassium salt). A mixture of 4-hydroxy-7-isopropyl-6-oxo-6,7-dihydro-thieno[2,3-b]pyridine-5-carboxylic acid (3-piperidin-1-yl-propyl)-amide hydrochloride (8, 2.35 g, 5.68 mmol), MTBE (23.5 mL), water (11.75 mL) and 1M KOH solution (5.6 mL) was stirred at room temperature for 20 minutes. The mixture still had insoluble hydrochloride salt. NaHCO₃ (0.49 gram) was added to above mixture and continued to stir for 0.5 hour. The mixture became a clear solution. The organic layer was separated. The aqueous layer was extracted with additional MTBE (20 mL). The combined organic layers were concentrated to dryness to give 4-hydroxy-7-isopropyl-6-oxo-6,7-dihydro-thieno[2,3-b]pyridine-5-carboxylic acid (3-piperidin-1-yl-propyl)-amide (2.17 grams) as free base.

The above free base (2.17 gram, 5.68 mmol) was dissolved in acetonitrile (23.5 mL) at 45-50 degrees Celsius To above solution was added a solution of KOH (0.43 gram, 6.53 mmol) in water (1.8 mL). The resulting mixture was stirred at 50 degrees Celsius for 0.5 hours, cooled to room temperature, then cooled at 0-5 degrees Celsius for 3 hours. The solid was filtered to give 3 grams of wet product which was dissolved into a mixture of acetonitrile (42 mL) and water (3 mL) at 50 degrees Celsius. The solution was filtered to remove insoluble solid. The filtrates were concentrated to a volume of 21 mL. Acetonitrile (21 mL) was added and concentrated to a volume of 21 mL. This process repeated twice. The residue was placed in ice-water bath for 3 hours, filtered and the filter cake was washed with acetonitrile (at 0 degrees Celsius, 2×6 mL), dried on rotavapor to give crystalline 4-hydroxy-7-isopropyl-6-oxo-6,7-dihydro-thieno[2,3-b]pyridine-5-carboxylic acid (3-piperidin-1-yl-propyl)-amide potassium salt (1.75 gram, yield: 74.2%; HPLC: 99.9%, KF: 1.19%) as white solid. The batch started with 84.5 grams of hydrochloride salt 8 converted to 76.3 grams of crystalline fine particle Form I of PRX-3140 potassium salt (yield: 90%; HPLC: 99.6%, KF: 0.66%). 1H NMR (300 MHz, DMSO-d₆) δ 10.79 (t, J=5.3 Hz, 1H), 7.13 (d, J=5.4 Hz, 1H), 6.80 (d, J=5.4 Hz, 1H), 3.19-3.11 (m, 2H), 2.70-2.40 (m, 6H), 1.62-1.30 (m, 15H).

Example 2—X-ray Diffraction of Crystalline PRX-3140 Potassium Salt Prepared at Two Scales. Crystalline fine particle Form I of PRX-3140 potassium salt samples of Examples 1 and a scale-up batch were used to identify differences in the crystalline structure of the two samples through standard x-ray diffraction (XRD) measurement. In both examples the same peaks were obtained shown in FIGS. 3A and 3B as well as Tables 1 and 2, respectively.

TABLE 1
XRD of Example 1 crystalline fine particle
Form I of PRX-3140 potassium salt

| Peak Value (degree) | Peak Intensity |
|---|---|
| 5.441 | 1087 |
| 11.875 | 457 |
| 12.994 | 304 |
| 13.730 | 460 |
| 15.919 | 696 |
| 16.286 | 501 |
| 16.520 | 553 |
| 17.089 | 509 |
| 19.428 | 254 |
| 19.879 | 172 |
| 20.331 | 536 |
| 20.648 | 160 |
| 21.266 | 524 |
| 21.601 | 566 |
| 22.303 | 1935 |
| 22.938 | 419 |
| 23.556 | 314 |
| 24.325 | 444 |
| 24.726 | 365 |
| 25.260 | 1325 |
| 25.829 | 930 |
| 26.347 | 163 |
| 27.433 | 441 |
| 28.553 | 237 |
| 29.137 | 212 |
| 29.923 | 619 |
| 30.792 | 165 |
| 32.613 | 306 |
| 33.081 | 481 |
| 34.435 | 382 |
| 34.853 | 223 |
| 35.789 | 246 |
| 36.440 | 274 |
| 36.891 | 172 |
| 37.276 | 212 |
| 39.264 | 321 |
| 39.649 | 228 |
| 40.050 | 283 |
| 40.651 | 160 |
| 41.069 | 308 |
| 41.738 | 204 |
| 42.456 | 314 |
| 43.225 | 209 |
| 43.793 | 280 |
| 44.746 | 327 |
| 44.930 | 339 |
| 45.297 | 391 |
| 45.581 | 474 |
| 46.283 | 252 |
| 46.751 | 160 |
| 46.985 | 173 |
| 48.222 | 243 |
| 48.773 | 181 |
| 48.924 | 164 |
| 49.308 | 176 |
| 49.676 | 165 |
| 50.578 | 228 |
| 52.617 | 190 |
| 53.937 | 172 |
| 54.355 | 175 |
| 54.806 | 169 |
| 56.059 | 160 |
| 58.048 | 161 |

TABLE 2
XRD of Scale-up batch of crystalline fine
particle Form I of PRX-3140 potassium salt

| Peak Value (degree) | Peak Intensity |
|---|---|
| 5.444 | 233 |
| 11.928 | 228 |
| 13.031 | 162 |
| 13.783 | 245 |
| 15.989 | 431 |
| 16.356 | 298 |
| 16.574 | 324 |
| 17.142 | 294 |
| 19.498 | 203 |
| 20.400 | 321 |
| 21.336 | 371 |
| 21.654 | 374 |
| 22.389 | 1222 |
| 23.007 | 280 |
| 23.609 | 247 |
| 24.378 | 342 |
| 24.812 | 295 |
| 25.330 | 810 |
| 25.898 | 637 |
| 26.734 | 161 |
| 27.486 | 264 |
| 28.622 | 203 |
| 29.207 | 191 |
| 29.993 | 394 |
| 30.611 | 241 |
| 32.683 | 215 |
| 33.151 | 338 |
| 34.438 | 269 |
| 35.507 | 160 |
| 35.842 | 189 |
| 36.159 | 160 |
| 36.527 | 206 |
| 37.312 | 200 |
| 38.432 | 161 |
| 39.368 | 320 |
| 40.136 | 232 |
| 41.173 | 247 |
| 41.624 | 163 |
| 41.824 | 186 |
| 42.526 | 294 |
| 43.144 | 171 |
| 43.295 | 197 |
| 43.796 | 213 |
| 43.947 | 220 |
| 44.849 | 298 |
| 45.668 | 369 |
| 46.303 | 217 |
| 46.955 | 192 |
| 48.342 | 240 |
| 49.027 | 167 |
| 49.361 | 196 |
| 49.762 | 160 |
| 49.929 | 160 |
| 50.648 | 205 |
| 50.965 | 160 |
| 51.818 | 161 |
| 52.737 | 165 |
| 53.957 | 160 |
| 54.241 | 160 |
| 54.408 | 160 |
| 54.892 | 204 |
| 56.079 | 160 |
| 58.836 | 160 |

Example 3—PRX-3140 Potassium Salt Photostability Study. Two samples of crystalline fine particle Form I of PRX-3140 potassium salt were prepared for photostability testing: (1) 1.5 grams spread evenly across a glass Petri dish and covered with a transparent quartz cover and (2) 1.5 grams spread evenly across a glass Petri dish and covered with aluminum foil. Samples were exposed to ICH photostability guideline Q1B=1.2 million lux hours of cool, white fluorescent light & 200 watt hours/m2 of UVA light at 40 degrees Celsius/75% RH. Samples were exposed for 2 days and tested using HPLC.

The control photostability sample of PRX-3140 potassium salt met the specification and showed HPLC results comparable to those obtained for the t=0 sample although an increase in water content from 0.900 w/w to 3.1% w/w was observed. The light exposure was performed in a cabinet set at 25 degrees Celsius/60% RH. The sample was notably lumpy and required grinding to obtain precise assay results.

TABLE 3
Photostability Study

| | | | Exposed |
|---|---|---|---|
| | t = 0 | Control | Sample |
| Appearance | Light brown solid | Pinkish solid | Orange |
| Water Content - KF | 0.9% w/w | 3.1% w/w | 4.3% w/w |
| Total impurities | 0.22% | 0.20% | 0.77% |
| Unknown (RRT 0.55) | 0.01% | 0.01% | 0.01% |
| Unknown (RRT 0.74) | 0.01% | 0.01% | 0.02% |
| Unknown (RRT 0.75) | 0.01% | 0.01% | 0.01% |
| Unknown (RRT 0.86) | nd | nd | 0.01% |
| Unknown (RRT 0.87) | 0.09% | 0.09% | 0.10% |
| Unknown (RRT 0.90) | nd | nd | 0.01% |
| Unknown (RRT 0.91) | nd | nd | 0.01% |
| Unknown (RRT 0.95) | 0.05% | 0.04% | 0.05% |
| Unknown (RRT 0.98) | 0.01% | 0.01% | 0.04% |
| Unknown (RRT 1.07) | nd | 0.01% | 0.06% |
| Unknown (RRT 1.12) | 0.01% | 0.01% | 0.02% |
| Unknown (RRT 1.22) | 0.01% | 0.01% | 0.01% |
| Unknown (RRT 1.50) | nd | nd | 0.02% |
| Unknown (RRT 1.54) | nd | nd | 0.01% |
| Unknown (RRT 1.57) | nd | nd | 0.01% |
| Unknown (RRT 1.63) | nd | nd | 0.05% |
| Unknown (RRT 1.72) | nd | nd | 0.04% |
| Unknown (RRT 1.72) | nd | nd | 0.02% |
| Unknown (RRT 1.75) | nd | nd | 0.04% |
| Unknown (RRT 1.76) | nd | nd | 0.03% |
| Unknown (RRT 1.79) | nd | nd | 0.01% |
| Unknown (RRT 1.83) | nd | nd | 0.16% |
| Unknown (RRT 2.01) | 0.01% | nd | nd |
| Unknown (RRT 2.06) | nd | nd | 0.01% |
| Unknown (RRT 2.08) | nd | nd | 0.01% |
| Unknown (RRT 2.16) | 0.01% | nd | 0.01% |
| Unknown (RRT 2.38) | 0.01% | nd | nd |

Key to table
RRT = Relative Retention Time,
nd = not detected

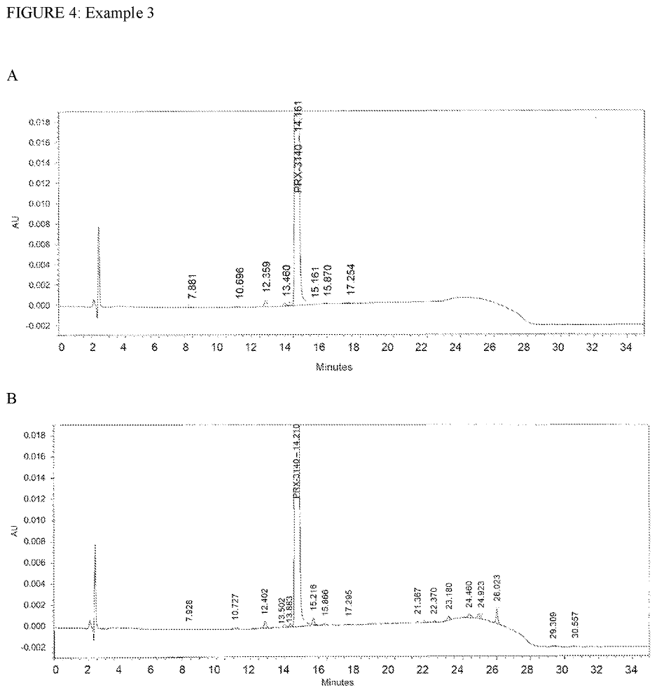

FIG. 4 shows the PRX-3140 potassium salt photostability study HPLC chromatograms (A) unexposed control sample and (B) PRX-3140 potassium salt photostability exposed sample. Changes were evident for crystalline fine particle Form I of PRX-3140 potassium salt after exposure to UVA and white light. The sample changed color particularly on the upper exposed surface of the material and there were changes apparent in the HPLC data. The illuminated photostability sample of PRX-3140 potassium salt met the USP stability specification of (1) total impurities not more than 2.000 area and (2) no single impurity greater than 0.50% area with a reported assay value of 94.20% w/w, which was below the specification limit. The single impurity profile met the specification (0.20) but significant changes were observed. There was an increase in the number of minor impurities, particularly in a region of the chromatogram between RRT 1.5 and 2.1. Total impurities increased from 0.20% in the control sample to 0.770 in the exposed sample. An impurity at RRT 1.83 which was not present before exposure to light was formed at 0.16% in the exposed sample. Other changes included a change in appearance of the material to a sticky solid and an increase in water content to 4.3% w/w.

Example 4—Crystalline PRX-3140 Potassium Salt Forced Degradation Study. Crystalline fine particle Form I of PRX-3140 potassium salt was subjected to a forced degradation study using acid, base, or hydrogen peroxide exposure for 24 hours. Samples were neutralized and analyzed using LC-UV-MS. Table 4 lists the LC-UV-MS conditions.

TABLE 4
LC-UV-MS conditions

| HPLC/MS | Agilent LC/MSD 6120B | |
|---|---|---|
| Source | Electrospray (ESI) | |
| Method | Gradient | |
| Diluent | 20/80 Methanol/Water | |
| Mobile Phase A | Water with 0.1% Formic Acid | |
| Mobile Phase B | Acetonitrile with 0.1% Formic Acid | |
| Gradient | 90/10 A/B -> 90/10 A/B (1 min) -> 40/60 A/B | |
| | (20 min) -> 10/90 A/B (30 min) -> 10/90 A/B | |
| | (35 min) -> 90/10 A/B (35.5 min) -> 90/10 A/B | |
| | (40 min) | |
| Column | Waters Atlantis T3, 3 μm, 150 × 4.6 mm | |
| Temp | 30 | degrees Celsius |
| Flow Rate | 1 | mL/min |
| Injection Volume | 10 | uL |
| UV Detection | 250, 220, and 320 nm | |
| Scan | 125 to 1000 m/z | |
| Step Size | 0.1 | m/z |
| Fragmentor | 150 | V |
| Gain | 5 | |
| Threshold | 100 | |
| Drying Gas | 325 | degrees Celsius |
| Capillary | 3000 | V |
| Nebulizer Gas | 7 | LPM |

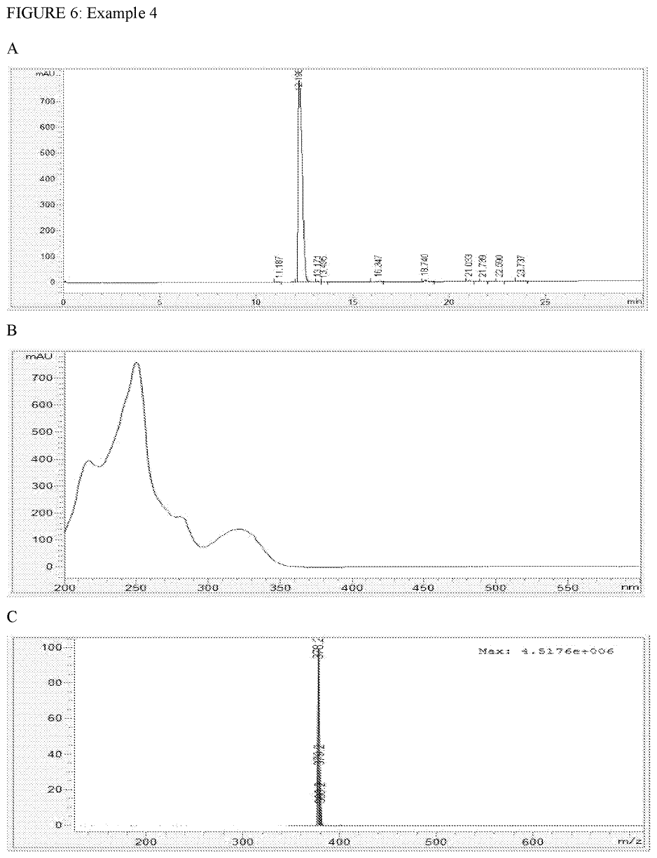

Control Sample: FIG. 6A is HPLC UV chromatogram of the crystalline fine particle Form I of PRX-3 140 potassium salt Standard (Control) at 250 nm. The UV spectra for the parent molecule is shown in FIG. 6B. Maximums are noted at 220, 250, and 320 nm. Chromatograms for all samples were collected at all three wavelengths with 250 nm being the primary wavelength based on lambda max. The starting material was 98.6% pure based on peak area at 250 nm. The mass spectra at 12.2 minutes for the parent molecule, PRX-3140, is shown in FIG. 6C. The protonated PRX-3140 molecular ion, [M+H]+, is noted at 378.2 m/z.

Sample Preparation: Crystalline fine particle Form I of PRX-3140 potassium salt, 50 mg, was weighed out into a 25 mL volumetric flask and taken to volume with water (2 mg/mL stock). One mL of stock was added to four, 4 mL vials. One mL of either 1N HCl, 1N NaOH, 3% H2O2, or DI Water (control) was added to the appropriate vial, mixed, and stored at room temperature for 24 hours. After storage, the samples of acid and base were neutralized by addition of 1 mL their counter solution. One mL of water was added to the peroxide sample and the control so that all samples had a final concentration of 0.67 mg/mL. Samples were analyzed using LC-UV-MS.

Table 5 lists a comparison for the control, acid, base, and peroxide samples showing percent peak area versus relative retention time (RRT) at 250 nm. While some minor changes can be noted between the control with the acid and base samples, the overall purity (peak area) of the parent molecule is just slightly higher for the acid and base sample. Some of the minor impurities appear to be reacting with the acid or base, potentially reforming to the parent. The greatest change in purity was noted in the peroxide sample where 2 major impurities were formed and the purity at 250 nm went from 98.6 to 80.6%.

TABLE 5
PRX-3140 Potassium Salt Forced Degradation Study

| RRT | Control | Acid | Base | H2O2 |
|---|---|---|---|---|
| 0.78 | | | | 8.49 |
| 0.82 | | | | 0.10 |
| 0.94 | | | 0.28 | |
| 0.99 | | 0.07 | | |
| 1.00 | 98.60 | 98.94 | 98.71 | 80.57 |
| 1.05 | <0.03 | <0.03 | <0.03 | 9.06 |
| 1.08 | 0.12 | 0.11 | 0.12 | 0.19 |
| 1.11 | 0.04 | 0.04 | | |
| 1.27 | | 0.04 | | 0.21 |
| 1.34 | 0.17 | 0.15 | 0.16 | 0.09 |
| 1.42 | | | | 0.32 |
| 1.53 | 0.42 | 0.43 | 0.42 | 0.42 |
| 1.71 | 0.23 | | | |
| 1.79 | 0.11 | 0.04 | 0.05 | 0.19 |
| 1.93 | 0.06 | | | |
| 2.51 | 0.06 | 0.06 | 0.05 | |
| 2.70 | 0.18 | 0.13 | 0.12 | 0.11 |

Acid Degradation Sample: Based on the purity peak area at 250 nm, the control was 98.6% and the acid sample was 98.9%. 1 N HCl for 24 hours had very little effect on PRX-3140 potassium salt.

Base Degradation Sample: Based on the purity peak area at 250 nm, the control was 98.6% and the base sample was 98.7%. 1 N NaOH for 24 hours had very little effect on PRX-3140 potassium salt.

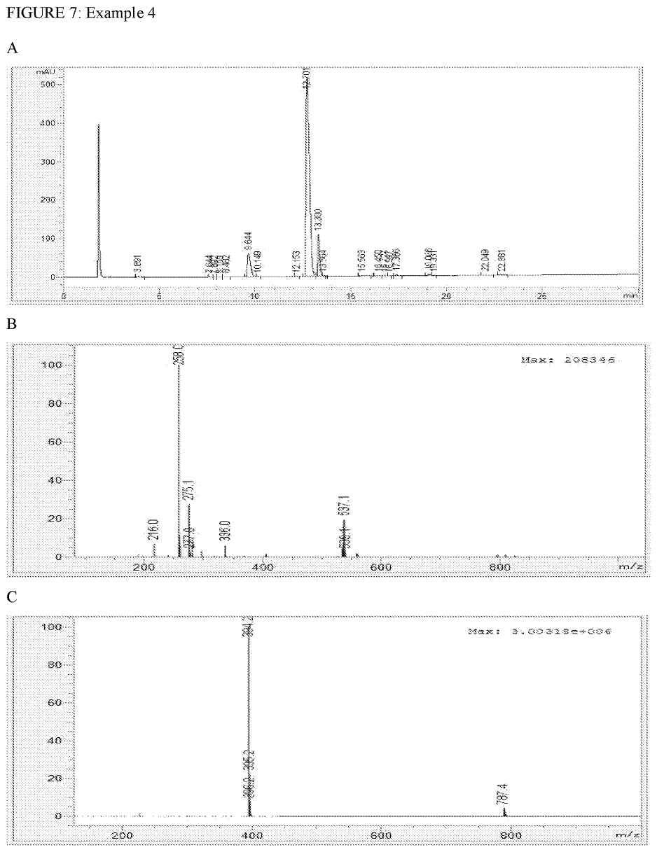

Peroxide Degradation Sample: The UV chromatogram for the peroxide degradation sample at 250 nm is shown in FIG. 7A full scale. Two major impurity peaks are noted at 9.6 and 13.3 minutes (RRT 0.78 and 1.05 respectively). Based on the purity peak area at 250 nm, the control was 98.6% and the peroxide sample was 80.6%. Hydrogen peroxide, 3%, for 24 hours reacted with PRX-3140 and formed 2 major degradants.

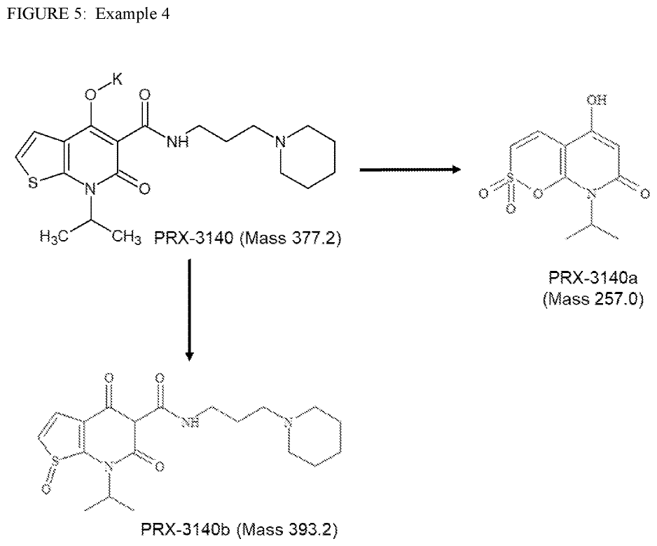

FIG. 7B is the mass spectra of PRX-3140a at 9.6 minutes (RRT 0.78). An [M+H]+ is noted at 258.0 m/z with an ammonium adduct [M+H+NH3]+ at 275.1, a sodium adduct [M+Na]+ at 280.1, and sodium dimer [2M+Na]+ at 537.1 m/z. It is noted that this is a fragile molecule and required the reduction of the Fragmentor Voltage from 150 down to 70V to see the protonated molecular ion. At the higher voltage, molecule fragments were noted at 216.0, 198.0, and 152.0. The proposed structure of PRX-3140a is Formula II: ##STR00002 ## 5-hydroxy-8-(methylethyl)-8-hydro-1,2-oxathiino[6.5-b]pyridine-2,2,7-trione, or 5-hydroxy-8-(propan-2-yl)-2H-2λ6-[1,2]oxathiino[6,5-b]pyridine-2,2,7(8H)-trione with the chemical formula of C10H11NO5S and is shown in FIG. 5.

FIG. 7C is the mass spectra of PRX-3140b at 13.3 minutes (RRT 1.05). An [M+H]+ is noted at 394.2 m/z with [2M+H]⁺ at 787.4 m/z. This compound is noted in the control, acid, and base samples but at a very low level (<0.03%). In the peroxide sample, this peak is 9.1%. The proposed structure of PRX-3140b is Formula III: ##STR00003 ## [7-(methylethyl)1,4,6-trioxo(5,7-dihydrothiopheno[2,3-b]pyridine-5-yl)]-N-(3-piperidylpropyl)carboxamide, or 1,4,6-trioxo-N-[3-(piperidin-1-yl)propyl]-7-(propan-2-yl)-4,5,6,7-tetrahydro-1H-1)4-thieno[2,3-b]pyridine-5-carboxamide with the chemical formula of C19H27N3O4S and is shown in FIG. 5.

Example 5—PRX-3140 30% Peroxide Degradation Study. Crystalline fine particle Form I of PRX-3140 potassium salt was subjected to a second forced degradation experiment using 30% hydrogen peroxide exposure for 24 hours. Sample was evaporated to dryness and analyzed using LC/UV/MS and NMR analysis. Two major degradants are noted in the LC/UV/MS.

Sample Preparation: 60 mg (55 μL) of 30% hydrogen peroxide (0.5 mmol) was added dropwise to a suspension of starting PRX-3140 potassium salt (208 mg, 0.5 mmol) in 5 mL of dry acetone at 0 degrees Celsius The resulting suspension was stirred under argon and warmed to RT spontaneously overnight (18 hours). The solvent is removed under vacuum at RT, then acetonitrile (5 mL) was added to the residue and evaporated to dryness at 40 degrees Celsius. The PRX-3140 30% peroxide sample residue weighed 300 mg.

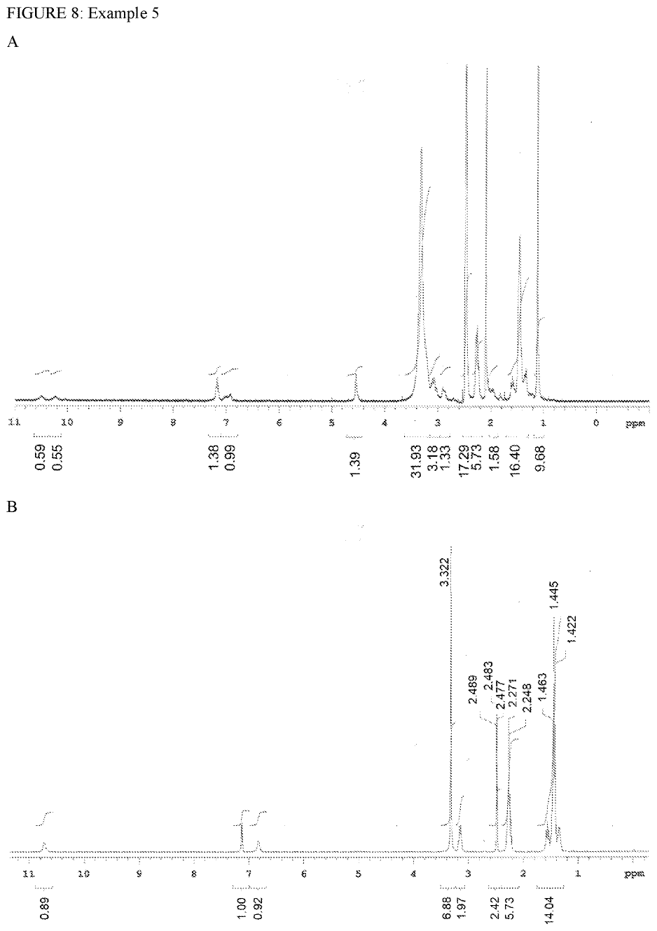

1H NMR of the crude 30% peroxide sample is shown in FIG. 8A. For comparison, 1H NMR of PRX-3140 potassium salt is shown in FIG. 8B.

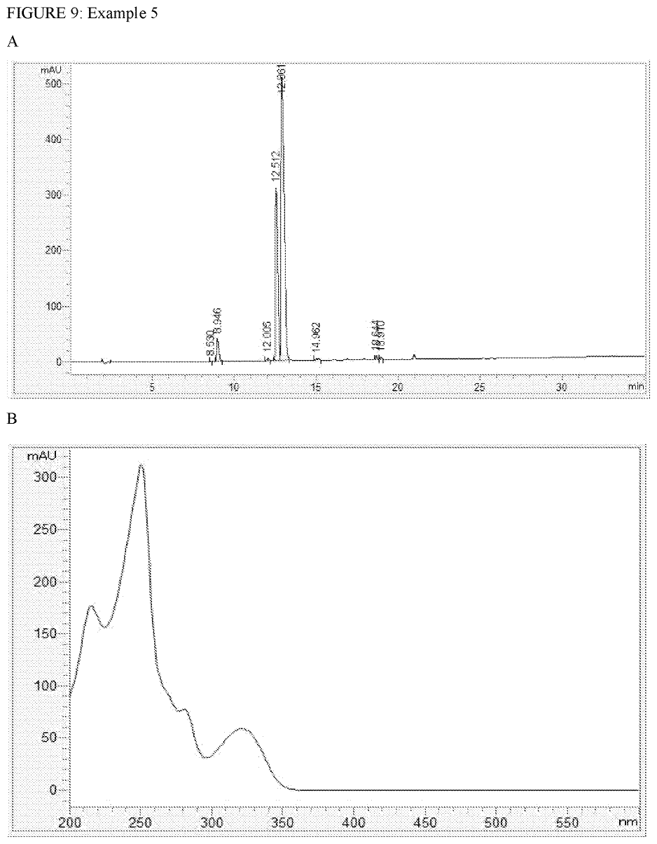

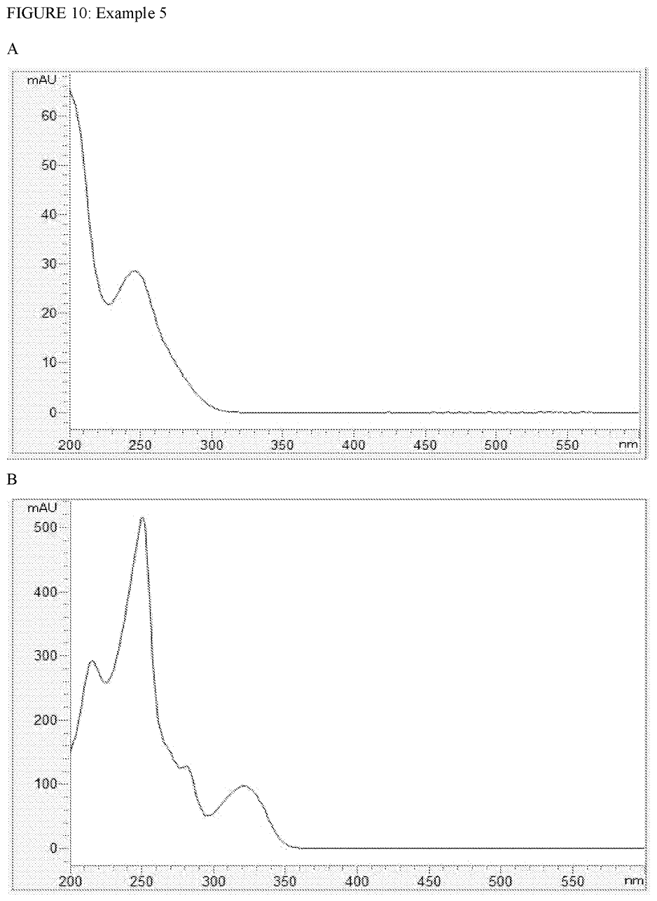

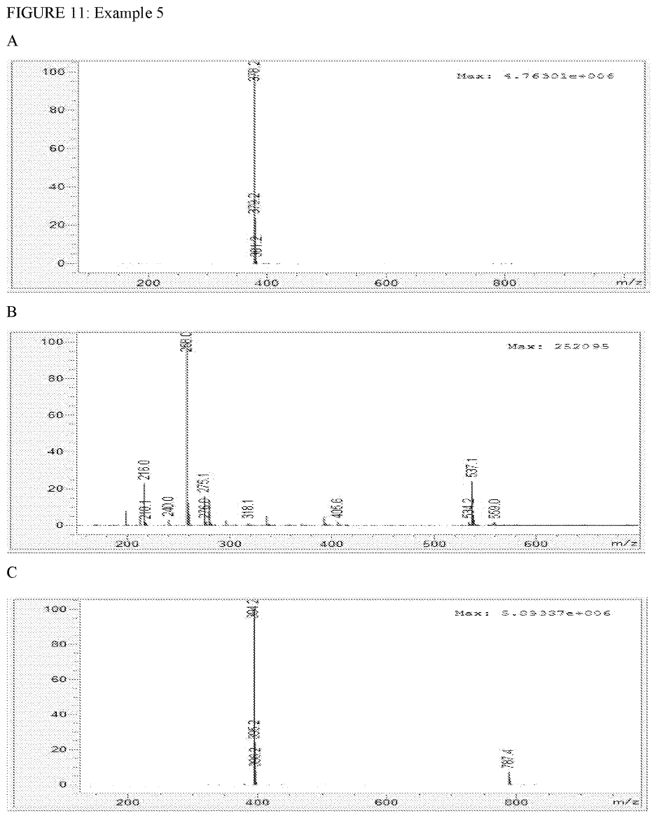

A solid portion of the PRX-3140 30% peroxide sample was submitted for LC/UV/MS analysis. Two milligrams of the sample was weighed into a 4 mL vial. Two mL of 20/80 methanol/water was added to the vial and vortexed vigorously. FIG. 9A is a HPLC chromatogram of the H2O2 sample at 250 nm. Three major peaks are noted, including the parent molecule at 12.5 minutes. A purity of 29.6% is noted for the parent molecule PRX-3140 following 18-hour exposure to 30% hydrogen peroxide. The UV spectra for the parent molecule at 12.5 min is shown in FIG. 9B. Maximums are noted at 220, 250, and 320 nm. Chromatograms for all samples were collected at all three wavelengths with 250 nm as the primary wavelength based on lambda max. FIG. 10A is the UV spectra for the peak at 8.9 minutes. In comparison with the parent molecule, the high-end absorption is absent (320 nm). FIG. 10B is the UV spectra for the peak at 12.9 minutes. The spectra are identical to the parent molecule noted in FIG. 9B. The mass spectra at 12.5 minutes for the parent molecule, PRX-3140, is shown in FIG. 11A. The protonated molecular ion, [M+H]+, is noted at 378.2 m/z. FIG. 11B is the mass spectra of PRX-3140a at 8.9 minutes (RRT 0.73 compared to 0.78 in Example 4). An [M+H]+ is noted at 258.0 m/z with [M+H+NH3]+ at 275.1, [M+Na]+ at 280.1, and [2M+Na]+ at 537.1 m/z. It is noted that this is a fragile molecule and required the reduction of the Fragmentor Voltage from 150V down to 70V to see the protonated molecular ion. At the higher voltage, molecule fragments were noted at 216.0, 198.0, and 152.0. A proposed structure of PRX-3140a is shown in FIG. 5. FIG. 11C is the mass spectra of the largest degradation product of PRX-3140b at 12.9 minutes, relative retention time (RRT) of 1.04 (compared to 1.05 in Example 4). The [M+H]+ of 394.2 m/z is potentially the parent molecule plus oxygen (+O). The proposed structure of PRX-3140B is shown in FIG. 5.

Example 6—PRX-3140 Excipient Compatibility Study. Excipient testing and compatibility studies were performed on the crystalline fine particle Form I of PRX-3140 potassium salt. We conducted excipient compatibility studies for binary blends (50:50 w/w) with the crystalline fine particle Form I of PRX-3140 potassium salt sample mixed with (1) Starch, pregelatinized, NF (Colorcon), (2) Microcrystalline Cellulose (Avicel PH-105, DuPont), (3) Magnesium Stearate NF (Avantor), (4) Stearic Acid, NF (Letco), (5) Lecithin, Granular Food Grade (Spectrum), (6) Polyethylene Glycol 3,350, USP (Dow), (7) HPB-Cyclodextrin (CTD), (8) Silicon Dioxide, FCC (Spectrum), and (9) Mannitol USP (Spectrum). Samples were analyzed by HPLC assay described in Example 4. Samples stored at 40 degrees Celsius/7500 RH and assay and purity tested by LC-UV-MS at 0, 30, 60, and 90 days shown in Table 6. At each timepoint powder samples were weighed and diluted at 50:50 methanol:DI water. Samples for HPLC analysis were filtered through a 0.45 micron PTFE filter for injection.

TABLE 6

| | | | Assay | % Purity | PRX-3140b | % |
|---|---|---|---|---|---|---|
| Sample | Timepoint | Color | PRX-3140 | PRX-3140 | RRT 1.06 | Impurities |
| Control | 0 | White | 98.8 | 98.69 | 0.03 | 1.31 |
| | 30 days | White | 100.9 | 98.74 | 0.03 | 1.26 |
| | 60 days | White | 99.5 | 98.74 | 0.03 | 1.26 |
| | 90 days | White | 100.3 | 98.63 | 0.04 | 1.27 |
| Starch | 0 | White | 100.0 | 98.79 | 0.02 | 1.21 |
| | 30 days | White- | 96.1 | 98.59 | 0.04 | 1.36 |
| | | Yellow | | | | |
| | 60 days | White- | 96.2 | 98.78 | 0.05 | 1.17 |
| | | Yellow | | | | |
| | 90 days | White- | 96.2 | 98.71 | 0.05 | 1.20 |
| | | Yellow | | | | |
| MCC | 0 | White | 100.0 | 98.73 | 0.03 | 1.27 |
| | 30 days | White- | 99.3 | 98.72 | 0.04 | 1.28 |
| | | Yellow | | | | |
| | 60 days | White- | 97.4 | 98.81 | 0.04 | 1.19 |
| | | Yellow | | | | |
| | 90 days | White- | 98.9 | 98.81 | 0.05 | 1.20 |
| | | Yellow | | | | |
| Magnesium | 0 | White | 100.0 | 98.76 | 0.03 | 1.24 |
| Stearate | 30 days | White | 82.2 | 98.85 | 0.05 | 1.15 |
| | 60 days | White | 83.5 | 98.93 | 0.05 | 1.17 |
| | 90 days | White | 89.4 | 98.91 | 0.05 | 1.09 |
| Stearic | 0 | White | 100.0 | 98.77 | 0.03 | 1.23 |
| Acid | 30 days | White- | 100.1 | 98.86 | 0.06 | 1.14 |
| | | Yellow | | | | |
| | 60 days | White- | 94.2 | 98.90 | 0.06 | 1.02 |
| | | Yellow | | | | |
| | 90 days | White- | 89.4 | 99.00 | 0.07 | 1.00 |
| | | Yellow | | | | |
| Lecithin | 0 | Tan | 100.0 | 98.76 | 0.03 | 1.24 |
| | 30 days | Tan | 99.1 | 98.37 | 0.49 | 1.59 |
| | 60 days | Tan-Brown | 92.9 | 98.08 | 0.67 | 1.85 |
| | 90 days | Tan-Brown | 94.9 | 97.84 | 1.09 | 2.12 |
| PEG 3350 | 0 | White | 100.0 | 98.78 | 0.02 | 1.22 |
| | 30 days | White- | 97.1 | 98.70 | 0.05 | 1.30 |
| | | Yellow | | | | |
| | 60 days | White- | 96.5 | 98.73 | 0.06 | 1.27 |
| | | Yellow | | | | |
| | 90 days | White- | 94.4 | 98.57 | 0.08 | 1.30 |
| | | Yellow | | | | |
| HPBCD | 0 | White | 100.0 | 98.76 | 0.02 | 1.24 |
| | 30 days | White | 96.4 | 98.76 | 0.03 | 1.24 |
| | 60 days | White | 94.1 | 98.69 | 0.03 | 1.29 |
| | 90 days | White | 94.7 | 98.75 | 0.03 | 1.15 |
| Silicon | 0 | White | 100.0 | 98.77 | 0.03 | 1.23 |
| Dioxide | 30 days | White | 93.2 | 98.74 | 0.03 | 1.26 |
| | 60 days | White | 84.6 | 98.84 | 0.03 | 1.16 |
| | 90 days | White | 85.2 | 98.89 | 0.04 | 1.10 |
| Mannitol | 0 | White | 100.0 | 98.77 | 0.02 | 1.23 |
| | 30 days | White | 96.1 | 98.77 | 0.03 | 1.23 |
| | 60 days | White | 96.2 | 98.81 | 0.03 | 1.17 |
| | 90 days | White | 96.2 | 98.57 | 0.03 | 1.20 |

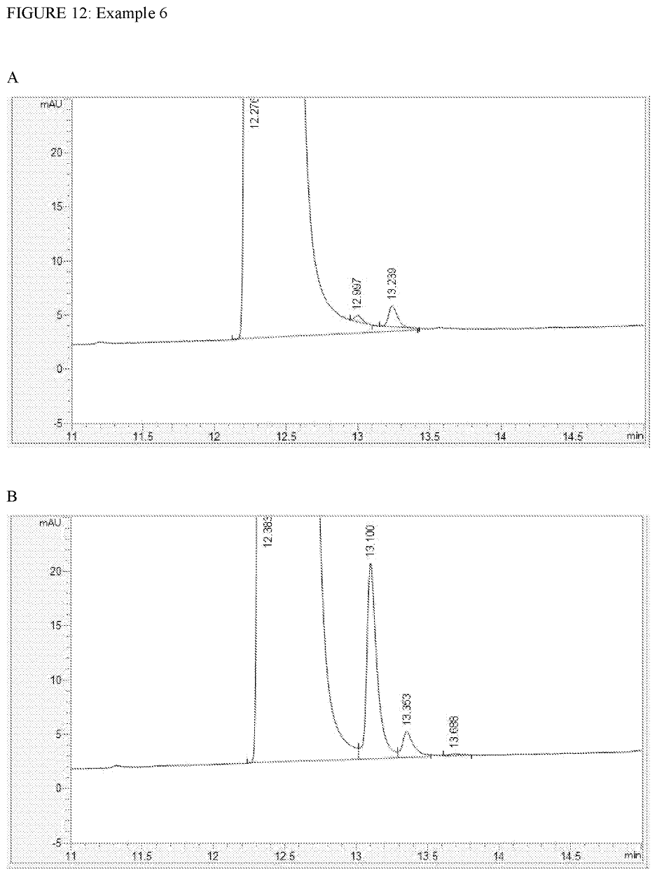

The PRX-3140:lecithin (50:50) sample degraded over the 90 day study and the PRX-3140a degradation product (RRT 1.06) increased to over 1%. FIG. 12A shows the PRX-3140:lecithin (50:50) Time 0 sample and FIG. 12B shows the 90 day sample demonstrating an increase of PRX-3140B (RRT 1.05) at 90 days at 40 degrees Celsius.

Example 7—Preparation of PRX-3140 Potassium Salt PDS. A mass of 10 g of crystalline fine particle Form I of PRX-3 140 potassium salt (Alchem Laboratories Corp.) and 10 g of Microcrystalline Cellulose (Avicel PH-105, DuPont) was mixed in 50 mL tube. The powder was then ground into a fine powder using a Retsch mill. Particles smaller than 600 microns were separated by sieving (30 mesh). The resulting white powder was obtained with a yield of >90% crystalline PRX-3140 potassium salt PDS powder, containing particles with a diameter less than 500 microns.

Example 8—Preparation of Immediate-Release Oral PRX-3140 Potassium Salt Capsules. An immediate-release oral dosage form (gelatin capsules) containing the crystalline fine particle Form I of PRX-3140 potassium salt particles prepared in Example 7 was dry mixed with additional Microcrystalline Cellulose (Avicel PH-105, DuPont) to achieve the correct capsule fill weight (400-500 mg) to achieve the desired dose. Clear gelatin #1 capsules were then filled with the mixture in a Torpac Profill 3700 machine to yield capsules containing 320 mg of PRX-3140 potassium salt PDS with 50 mg PRX-3140. Samples were taken to verify loading uniformity, content uniformity, and dissolution time.

Example 9—Dissolution of Immediate-Release Oral PRX-3140 Potassium Salt. Capsules from Example 8 were exposed to acidic buffer for 60 minutes to mimic the stomach environment. To demonstrate dissolution, more than 75% release of PRX-3140 in solution at 15 to 30 minutes in acidic buffer was tested using a USP dissolution apparatus and HPLC.

Example 10—Preparation of Sustained-Release Oral PRX-3140 Potassium Salt PDS. Crystalline fine particle Form I of PRX-3140 potassium salt (Alchem Laboratories Corp.), microcrystalline cellulose (MCC) and different sustained-release polymers (1) Methocel E4M (Mn 86,000, 2% in water viscosity ~4,000 mPa·s), (2) Methocel K100M (Mn=220,000, 2% viscosity; ~100,000 mPa·s), (3) Kollidon SR (polyvinyl acetate 80%, weight average molecular weight of about 450,000 daltons/polyvinyl pyrrolidone, weight average molecular weight of about 55,000 daltons), and (4) xanthan gum were weighed (100 g total each), mixed and ground in a glass mortar and pestle. Particles smaller than 500 microns were separated by sieving (35 mesh). The ratio of 3.3% was used to fill Size 1 capsules with 300 mg fill weight to obtain 10 mg PRX-3140/capsule. The % weight for each ingredient is shown in Table 7 below.

TABLE 7

| | PRX- | | | | | |
|---|---|---|---|---|---|---|
| | 3140 | MCC | E4M | K100M | Kollidon | Xanthan |
| Formulation | (wt %) | (wt %) | (wt %) | (wt %) | (wt %) | (wt %) |
| 1 | 3.3 | 96.7 | — | — | — | — |
| 2 | 3.3 | 48.3 | 48.3 | — | — | — |
| 3 | 3.3 | 48.3 | — | 48.3 | — | — |
| 4 | 3.3 | 48.3 | — | — | 48.3 | — |
| 5 | 3.3 | 48.3 | — | — | — | 48.3 |
| 6 | 3.3 | 66.3 | 30.4 | — | — | — |
| 7 | 3.3 | 66.3 | — | 30.4 | — | — |
| 8 | 3.3 | 66.3 | — | — | 30.4 | — |
| 9 | 3.3 | 66.3 | — | — | — | 30.4 |

Example 11—X-ray Diffraction of Sustained-Release Oral PRX-3140 Potassium Salt. Formulation 7 and 9 sustained-release PRX-3140 potassium salt particles of Example 10 were used to identify differences in the crystalline structure of the two sustained-release PRX-3140 powder samples through standard x-ray diffraction (XRD) measurement. In both examples the same peaks were obtained shown in FIGS. 13A and 13B as well as Tables 8 and 9, respectively.

TABLE 8
XRD of Formulation 7

| Peak Value (degree) | Peak Intensity |
|---|---|
| 5.352 | 1974 |
| 11.652 | 347 |
| 11.702 | 335 |
| 12.789 | 203 |
| 13.557 | 388 |
| 15.830 | 964 |
| 16.214 | 680 |
| 16.900 | 509 |
| 17.802 | 171 |
| 18.119 | 161 |
| 19.022 | 240 |
| 19.406 | 234 |
| 20.175 | 587 |
| 21.178 | 642 |
| 21.478 | 331 |
| 22.180 | 1495 |
| 24.252 | 233 |
| 25.172 | 935 |
| 25.740 | 674 |
| 27.344 | 369 |
| 28.430 | 164 |
| 29.801 | 306 |
| 29.851 | 352 |
| 30.419 | 191 |
| 30.486 | 237 |
| 32.976 | 309 |
| 34.296 | 228 |
| 39.226 | 209 |
| 39.426 | 195 |
| 39.627 | 164 |
| 44.640 | 216 |
| 45.259 | 172 |
| 45.492 | 173 |

TABLE 9
XRD of Formulation 9

| Peak Value (degree) | Peak Intensity |
|---|---|
| 5.177 | 3550 |
| 11.635 | 802 |
| 12.755 | 435 |
| 13.490 | 639 |
| 15.696 | 1123 |
| 16.047 | 492 |
| 16.281 | 667 |
| 16.866 | 620 |
| 18.437 | 206 |
| 20.125 | 493 |
| 21.044 | 619 |
| 21.378 | 586 |
| 22.097 | 2126 |
| 22.715 | 331 |
| 23.350 | 196 |
| 24.085 | 252 |
| 24.520 | 263 |
| 25.038 | 1249 |
| 25.623 | 842 |
| 26.141 | 165 |
| 27.210 | 256 |
| 28.313 | 162 |
| 28.915 | 214 |
| 29.700 | 463 |
| 30.302 | 229 |
| 32.875 | 241 |
| 34.179 | 189 |
| 34.329 | 233 |
| 36.234 | 174 |
| 44.657 | 244 |
| 45.058 | 162 |
| 45.409 | 252 |

The Formulation 7 and 9 XRD baseline demonstrated a characteristic profile of Microcrystalline Cellulose and cellulose samples in general.

Example 12—Sustained-Release Oral PRX-3140 Potassium Salt Excipient Compatibility Study. Excipient testing and compatibility studies were performed on the crystalline fine particle Form I of PRX-3140 potassium salt mixed with sustained-release polymers from Example 10. Samples stored at 40 degrees Celsius/75% RH and assay and purity tested by LC-UV-MS at 30 days. At each timepoint powder samples were weighed and diluted with 20:80 methanol:DI water. Samples for HPLC analysis were filtered through a 0.45 micron PTFE filter for injection. The Samples stored at 40 degrees Celsius/75% RH and assay and purity tested by LC-UV-MS at 30 days shown in Table 10.

TABLE 10

| | | | % Purity | PRX- | % |
|---|---|---|---|---|---|
| Sample | Timepoint | Color | PRX-3140 | 3140b | Impurities |
| Control | 30 days | White | 98.71 | 0.11 | 1.29 |
| Formulation 1 | 30 days | White | 98.69 | 0.11 | 1.31 |
| Formulation 6 | 30 days | White | 98.63 | 0.14 | 1.37 |
| Formulation 7 | 30 days | White | 99.11 | 0.12 | 0.89 |
| Formulation 8 | 30 days | White | 98.73 | 0.11 | 1.26 |
| Formulation 9 | 30 days | White | 97.45 | 0.10 | 2.55 |

The Formulation 9 xanthan gum sample degraded slightly over the 30 day study (2.55% impurities) but the PRX-3140b degradation product (RRT 1.08) did not increase. Impurity peaks are most likely due to the xanthan gum impurities.

Example 13—Preparation of Sustained-Release Oral PRX-3140 Potassium Salt Capsules. Sustained-release oral dosage forms containing the crystalline fine particle Form I of PRX-3140 potassium salt particles prepared in Example 10 were dry mixed with additional Microcrystalline Cellulose (Avicel PH-105, DuPont) to achieve the correct capsule fill weight to achieve the desired dose. White gelatin #1 capsules were then filled with the mixture in a Torpac Profill 3700 machine to yield capsules containing 200-250 mg of PRX- 3140 potassium salt PDS with 10 mg PRX-3140. Samples were taken to verify loading uniformity, content uniformity, and dissolution time.

Example 14—Dissolution of Sustained-Release Oral PRX-3140 Potassium Salt. Capsules (n=10 each) from Example 13 were placed in a USP dissolution Type 1 basket and sampled for 24 hours in de-ionized water at 37 degrees Celsius. At each timepoint, samples for HPLC analysis were filtered through a 0.45 micron PTFE filter for injection. The % release for each formulation is shown in Table 11 below and FIG. 14.

TABLE 11

| | Drug in | Formulation | Formulation | Formulation | Formulation | Formulation |
|---|---|---|---|---|---|---|
| Hours | Capsule | 2 | 6 | 7 | 8 | 9 |
| 0 | 0% | 0% | 0% | 0% | 0% | 0% |
| 1 | 71.3% | 7.6% | 16.1% | 9.5% | 42.6% | 3.8% |
| 2 | 80.1% | 13.9% | 29.3% | 16.8% | 63.6% | 7.2% |
| 3 | 88.7% | 19.8% | 38.5% | 23.8% | 71.1% | 10.7% |
| 4 | 89.8% | 24.1% | 46.1% | 29.3% | 72.9% | 17.7% |
| 5 | 89.8% | 29.4% | 52.6% | 35.9% | 78.5% | 24.7% |
| 6 | 92.5% | 35.6% | 59.2% | 39.3% | 81.6% | 26.8% |
| 9 | 93.8% | 44.9% | 66.0% | 49.8% | 84.7% | 38.7% |
| 12 | 95.7% | 56.4% | 76.2% | 62.4% | 89.3% | 55.3% |
| 18 | 97.5% | 68.3% | 86.4% | 74.8% | 93.9% | 70.5% |
| 24 | 100.0% | 80.2% | 100.0% | 87.2% | 100.0% | 85.7% |

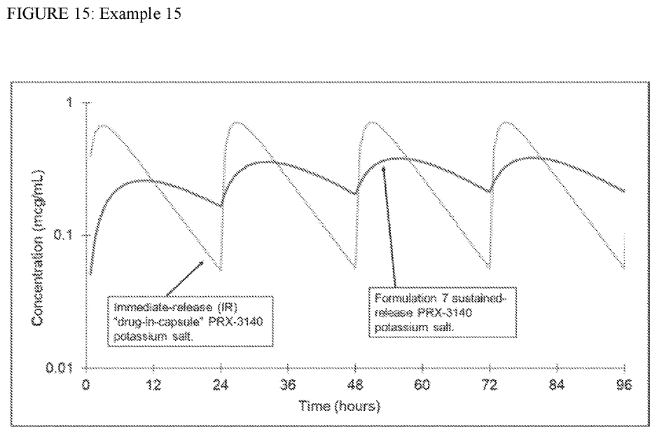

Example 15—Pharmacokinetics of Immediate and Sustained-Release Capsules. A Phase I, single dose escalation study under fasting conditions was conducted in male volunteers with PRX-3140 potassium salt and pharmacokinetic data (PRX-3140 acid) were obtained at 5 and 50 mg dose levels. Healthy volunteers were initially enrolled with six subjects completing all treatments. A second Phase I, open label multiple dose escalation study was conducted with PRX-3140 potassium salt and pharmacokinetic data were obtained up to 14 days at 10, 30, 100 and 200 mg dose levels administered once-a-day. Serum concentrations of PRX-3140 acid were determined using a validated liquid chromatography coupled with tandem mass spectrometry (LC/MS/MS) method. The calculated mean serum concentrations of PRX-3140 immediate-release “drug-in-capsule” at each time point and PK parameters are summarized below in Table 12 and graph of the fitted mean PRX-3140 acid serum concentration versus time at each dose level is shown in FIG. 15. Parameters such as Cmax, Tmax, AUC24, and T½ were estimated.

TABLE 12
Mean serum concentrations and calculated PK parameters for immediate-
release (IR) “drug-in-capsule” PRX-3140 potassium salt.

| | 5 | 10 | 30 | 50 | 100 | 200 |
|---|---|---|---|---|---|---|
| | mg | mg | mg | mg | mg | mg |
| | (ng/ | (ng/ | (ng/ | (ng/ | (ng/ | (ng/ |
| Hours | mL) | mL) | mL) | mL) | mL) | mL) |
| 0 | 0 | 0 | 0 | 0 | 0 | 0 |
| 0.5 | 4.3 | 11.1 | 22.5 | 147.9 | 262.3 | 680.7 |
| 1 | 12.0 | 37.6 | 67.1 | 308.2 | 646.0 | 742.3 |
| 1.5 | 11.6 | 54.4 | 82.2 | 281.8 | 688.0 | 669.7 |
| 2 | 13.2 | 52.4 | 74.1 | 254.2 | 602.8 | 710.0 |
| 4 | 9.1 | 37.6 | 50.5 | 167.4 | 338.0 | 395.8 |
| 6 | 7.6 | 37.6 | 50.5 | 110.3 | 338.0 | 395.8 |
| 8 | 4.2 | 19.4 | 33.1 | 96.8 | 205.8 | 177.2 |
| 12 | 3.1 | 12.9 | 18.3 | 58.7 | 129.4 | 86.1 |
| 16 | 1.1 | 8.2 | 12.2 | 48.0 | 94.7 | 59.1 |
| 24 | 0.6 | 5.7 | 8.7 | 38.2 | 70.7 | 34.2 |

Day 1

| | | | | | | |
|---|---|---|---|---|---|---|
| Tmax (h) | 2.0 | 1.5 | 1.5 | 1.0 | 1.5 | 1.0 |
| Cmax (ng/mL) | 13.2 | 55 | 76 | 308.2 | 641 | 996 |
| AUC24 (ng/mL*h) | 64 | 364 | 514 | 1718 | 3766 | 4243 |

Steady-state

| | | | | |
|---|---|---|---|---|
| Tmax (h) | 1.5 | 1.5 | 1.5 | 1.5 |
| Cmax (ng/mL) | 83.1 | 163.1 | 746.4 | 1602 |
| Cmin (ng/mL) | 13.7 | 24.9 | 106.2 | 165.8 |
| Cavg (ng/mL) | 30.5 | 59.3 | 245.4 | 537.0 |
| Fluctuation % | 369 | 448 | 320 | 307.2 |
| T½ (h) | 12.7 | 12.7 | 10.2 | 8.6 |

The PK data suggested that immediate-release (TR) “drug-in-capsule” PRX-3140 was absorbed at a relatively rapid rate (Tmax 1 to 2 hours) and the exposure showed moderate to high intersubject variability. The Cmax values at the doses of 30 to 200 mg were less variable than those at the lower doses studied (5 and 10 mg). Following multiple oral dose administration of 10, 30, 100, or 200 mg immediate-release “drug-in-capsule” PRX-3140 for 14 days, mean exposures (Cmax and AUC24) of PRX-3140 appeared to increase in a dose proportional manner. Absorption was moderately rapid, with Tmax ranging from 1 to 2 hours. Dose-related increases in the mean trough serum concentration increases were evident throughout the treatment period and appeared to reach steady state by approximately Day 7 for the 10, 30, and 100 mg cohorts and Day 14 for the 200 mg immediate-release “drug-in-capsule” PRX-3 140 cohort.

The calculated mean serum concentrations of Formulation 7 PRX-3140 sustained-release (SR) capsules at each time point and PK parameters are summarized below in Table 13 and logarithmic graph of the fitted mean PRX-3140 acid serum concentrations versus time shown in FIG. 15. Parameters such as Cmax, Tmax, AUC24, and T½ were estimated.

TABLE 13
Calculated mean serum concentrations and PK parameters for
Formulation 7 sustained-release PRX-3140 potassium salt.

| | 5 | 10 | 30 | 50 | 100 | 200 |
|---|---|---|---|---|---|---|
| | mg | mg | mg | mg | mg | mg |
| Day 1 | (ng/ | (ng/ | (ng/ | (ng/ | (ng/ | (ng/ |
| Hours | mL) | mL) | mL) | mL) | mL) | mL) |
| 0 | 0 | 0 | 0 | 0 | 0 | 0 |
| 0.5 | 2.4 | 4.7 | 14.2 | 23.6 | 47.2 | 94.4 |
| 1 | 4.5 | 8.9 | 26.7 | 44.6 | 89.1 | 178.3 |
| 1.5 | 6.3 | 12.6 | 37.9 | 63.1 | 126.2 | 252.5 |
| 2 | 7.9 | 15.9 | 47.7 | 79.5 | 158.9 | 317.9 |
| 4 | 12.6 | 25.3 | 75.8 | 126.3 | 252.7 | 505.3 |
| 8 | 12.6 | 25.3 | 75.8 | 126.3 | 252.7 | 505.3 |
| 12 | 16.0 | 32.0 | 95.9 | 159.8 | 319.6 | 639.2 |
| 16 | 15.2 | 30.4 | 91.1 | 151.8 | 303.5 | 607.1 |
| 24 | 12.8 | 25.7 | 77.0 | 128.3 | 256.6 | 513.1 |

Day 1

| | | | | | | |
|---|---|---|---|---|---|---|
| Tmax (h) | 8.7 | 8.7 | 8.7 | 8.7 | 8.7 | 8.7 |
| Cmax (ng/mL) | 16.0 | 32.1 | 96.2 | 160 | 321 | 642 |
| AUC24 (ng/mL*h) | 364 | 769 | 2308 | 3846 | 7692 | 15385 |

Steady-state

| | | | | |
|---|---|---|---|---|
| Tmax (h) | 7.6 | 7.6 | 7.6 | 7.6 |
| Cmax (ng/mL) | 38.7 | 116 | 387.2 | 774.4 |
| Cmin (ng/mL) | 21.4 | 64.3 | 214.2 | 428.5 |
| Cavg (ng/mL) | 32.1 | 96.1 | 321.1 | 641.1 |
| Fluctuation % | 54 | 54 | 54 | 54 |
| T½ (h) | 5.3 | 5.3 | 5.3 | 5.3 |

The calculated PK data suggested that the sustained-release Formulation 7 PRX-3140 dosage form was absorbed more slowly (Tmax 8.7 hours). The Cmax values for the sustained-release Formulation 7 PRX-3140 dosage form was lower compared to similar doses of the immediate-release “drug-in-capsule” PRX-3140 shown above in Table 13. Following multiple oral dose administration of Formulation 7 sustained-release PRX-3140 at 10, 30, 100, or 200 mg for 14 days, mean exposures (AUC24) of PRX-3140 acid appeared to increase in a dose proportional manner. Dose-related increases in the steady-state serum concentration were proportional to dose levels and peak to trough fluctuation 0% values were reduced due to slower absorption following once-a-day dosing of the sustained-release Formulation 7 PRX-3140 dosage form. The results from this study show that the SR dosage form shows a higher AUC24 (systemic exposure) and less % fluctuation of PRX-3140 acid at steady state when compared to the TR dosage form. According to the present invention, the SR dosage forms achieve a calculated average concentration at steady state (Cavg) of PRX-3140 free acid of at least 1 ng/ml at a dose of then 1 mg PRX-3140.

Example 16—Enteric-Coated Sustained-Release Oral PRX-3140 Potassium Salt Capsules. An enteric-coated sustained-release oral dosage form (gelatin capsules) containing the Formulation 7 PRX-3140 dosage form prepared in Example 13 was prepared as described below. Alternatively, Enprotect® (Capsugel) capsules may be used. Enteric coating of capsules or tablets of the present example was performed in a rotating-pan coater with sprayer attachment. The spray head was supplied with dry nitrogen gas feed and a peristaltic pump driven liquid feed. The coating was comprised of Eudragit L100 dissolved in a 97%/3% isopropanol/water solution. Samples were taken to verify loading uniformity, coating uniformity, content uniformity, and dissolution time.

Example 17—Preparation of Sustained Release Tablets of PRX-3140 Potassium Salt. Tableting of the blended mixtures may be performed by direct compression, dry granulation or wet granulation. To reduce the potential for degradation of PRX-3140, especially in solution phase, directly compression method was used to prepare tablets. The blended mixture was collected and directly dry compressed using a tablet press (Lumac Single-punch Model TDP-30) to form matrix tablets with sustained release agent. Enteric-coating of sustained Release Tablets of crystalline PRX-3140 Potassium Salt tablets under similar conditions as Example 16 were used to demonstrate lag-time of dissolution under acidic dissolution conditions.

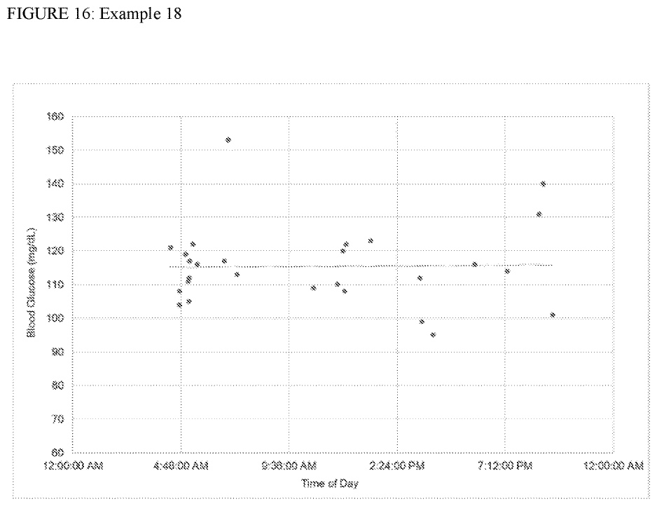

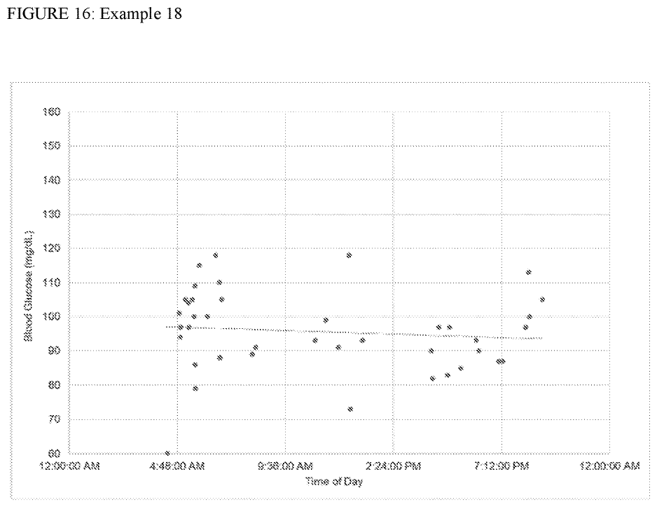

Example 18—Blood Glucose Levels of Controls vs. Sustained-Release Oral PRX-3140 Potassium Salt Capsules. A Phase I, single dose escalation study under fed conditions was conducted in male volunteers with PRX-3140 potassium salt at the 15 mg dose level. The mean blood glucose level decrease (−19.5%) was noted following administration of 15 mg PRX-3140 potassium salt orally compared to control values. To further evaluate the effect of oral crystalline PRX-3140 potassium salt sustained-release formulations, glucose levels were collected in patients and plotted vs. time of day shown in FIG. 16 (control blood glucose levels) and FIG. 17 (blood glucose levels following administration of 10 mg Formulation 7 sustained-release oral PRX-3140 potassium salt capsules. The calculated mean glucose concentrations of controls and Formulation 7 PRX-3140 sustained-release capsules at 10 mg, 30 mg, or 100 mg/day, as well as standard deviation, minimum and maximum glucose levels are summarized below in TABLE 14.

TABLE 14
Calculated mean glucose concentrations of controls and
Formulation 7 PRX-3140 sustained-release capsules, standard
deviation, minimum and maximum glucose levels.

| | Mean | Standard | Minimum | Maximum |
|---|---|---|---|---|
| | (mg/dL) | Deviation | (mg/dL) | (mg/dL) |
| Controls (n = 27) | 115.5 | 12.2 | 95 | 153 |
| 10 mg (n = 40) | 95.7 | 19.4 | 60 | 118 |
| 30 mg (n = 19) | 100.3 | 16.0 | 65 | 129 |
| 100 mg (n = 15) | 93.1 | 15.4 | 63 | 123 |

The study was also conducted to examine the effects of PRX-3140 sustained-release capsule administration on the subjects' fasting and postprandial blood glucose concentrations. Subjects were monitored daily for vital signs (including blood pressure, heart rate, respiratory rate, and temperature) and adverse events. In addition, blood was drawn to assess glucose concentration. Glucose was tested within 60 minutes prior to meals in the morning and throughout the day at different timepoints. All three Formulation 7 PRX-3140 sustained-release capsule groups (10 mg, 30 mg, and 100 mg) had similar self-monitored blood glucose profiles and similar mean average daily blood glucose concentrations at baseline (115.5 mg/dL, n=27). The mean average blood glucose concentrations decreased for all three Formulation 7 PRX-3140 sustained-release capsule treatment groups (93.1 to 100.3 mg/dL, n=15 to 40).

1. A sustained release oral dosage form in the form of a tablet or capsule comprising:
(a) a compound of Formula I: 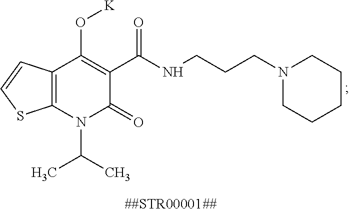
(b) microcrystalline cellulose; and
(c) at least one sustained-release polymer chosen from:
i. hydroxy-propyl-methyl-cellulose having a number average molecular weight of about 86,000 to about 220,000 daltons;
ii. a polymer mixture comprising polyvinyl acetate and polyvinyl pyrrolidone, wherein the polyvinyl acetate has a weight average molecular weight of about 450,000 daltons, wherein the polyvinyl pyrrolidone has a weight average molecular weight of about 55,000 daltons, and wherein the concentration of the polyvinyl acetate in the polymer mixture is about 80% by weight; and
iii. xanthan gum;
wherein greater than 90% by weight of the compound of Formula I is the crystalline form of Form I characterized by an x-ray powder diffraction pattern comprising major peaks at 22.3+/−0.3 degrees, 25.3+/−0.3 degrees, and 5.4+/−0.3 degrees two theta, and
wherein release of the compound of Formula I from the oral dosage form defined by in vitro dissolution is at most 50% as measured by reverse-phase high performance liquid chromatography (HPLC) at the 2 hour time point and at least 50% as measured by HPLC at the 12 hour time point, wherein the dissolution of the oral dosage form is performed according to general procedures of the United States Pharmacopeia (USP) II dissolution test (paddle method) or USP I (basket method) in water or simulated gastric fluid at pH 1.2 from hours 0 to 2 and in simulated intestinal fluid at pH 6.8 after 2 hours and a rotation of 50 revolutions per minute.

2. The sustained release oral dosage form of claim 1, wherein the at least one sustained-release polymer is the hydroxy-propyl-methyl-cellulose.

3. The sustained release oral dosage form of claim 1, wherein the at least one sustained-release polymer is the hydroxy-propyl-methyl-cellulose having a number average molecular weight of about 86,000 daltons.

4. The sustained release oral dosage form of claim 1, wherein the at least one sustained-release polymer is the hydroxy-propyl-methyl-cellulose having a number average molecular weight of about 220,000 daltons.

5. The sustained release oral dosage form of claim 1, wherein the at least one sustained-release polymer is the polymer mixture comprising polyvinyl acetate and polyvinyl pyrrolidone.

6. The sustained release oral dosage form of claim 1, wherein the at least one sustained-release polymer is xanthan gum.

7. The sustained release oral dosage form of claim 1, wherein the oral dosage form is in the form of a capsule.

8. The sustained release oral dosage form of claim 1, wherein the oral dosage form is in the form of a tablet.

9. The sustained release oral dosage form of claim 8, further comprising a tablet coating.

10. The sustained release oral dosage form of claim 1, wherein the weight ratio of microcrystalline cellulose to the at least one sustained-release polymer is between about 1:3 and about 3:1.

11. The sustained release oral dosage form of claim 10, wherein the weight ratio of microcrystalline cellulose to the at least one sustained-release polymer is between about 1:1 and about 3:1.

12. The sustained release oral dosage form of claim 1, wherein the oral dosage form comprises between about 2 weight % and about 10 weight % of the compound of Formula I.

13. The sustained release oral dosage form of claim 12, wherein the oral dosage form comprises between about 2 weight % and about 5 weight % of the compound of Formula I.

14. The sustained release oral dosage form of claim 13, wherein the oral dosage form comprises about 3.3 weight % of the compound of Formula I.

15. The sustained release oral dosage form of claim 1, wherein the oral dosage form further comprises:
(a) a compound of Formula II: 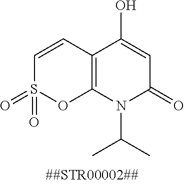 [SMILES: CC(C)n1c2c(c(O)cc1=O)C=CS(=O)(=O)O2]
(b) a compound of Formula III:

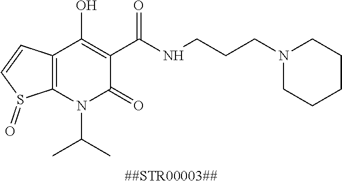 [SMILES: CC(C)n1c2c(c(O)c(C(=O)NCCCN3CCCCC3)c1=O)C=CS2=O]

16. The sustained release oral dosage form of claim 15, wherein the oral dosage form comprises less than 0.5 weight % of the compound of Formula II, if present, and less than 0.5 weight % of the compound of Formula III, if present.

17. The sustained release oral dosage form of claim 1, wherein the oral dosage form comprises 0.1 mg to about 250 mg of the compound of Formula I.

18. A method for treating a disease in a human in need thereof comprising administering to the human at least once daily a therapeutically effective amount of the sustained release oral dosage form of claim 1 to achieve an average concentration at steady state (Cavg) of at least 1 ng/ml of PRX-3140 free acid in the blood.

19. The method of claim 18, wherein the disease is type 2 diabetes.

20. The method of claim 18, wherein the disease is type 1 diabetes.

21. The method of claim 18, wherein the method further comprises administering to the human a glucocorticoid therapy.

22. The method of claim 18, wherein the disease is obesity.
